SEQUENCE LISTING (24 sequences)

SEQ ID NO: 1 - DNA, 21 bp, Artificial sequence
  Primer MYBL2
agcaagtgcaaggtcaaatgg

SEQ ID NO: 2 - DNA, 20 bp, Artificial sequence
  Primer MYBL2
ctgtccaaactgcctcacca

SEQ ID NO: 3 - DNA, 19 bp, Artificial sequence
  Primer CCNA2
gaagacgagacgggttgca

SEQ ID NO: 4 - DNA, 20 bp, Artificial sequence
  Primer CCNA2
ccaaggaggaacggtgacat

SEQ ID NO: 5 - DNA, 22 bp, Artificial sequence
  Primer STK6
tcttccaggaggaccactctct

SEQ ID NO: 6 - DNA, 21 bp, Artificial sequence
  Primer STK6
tgcatccgaccttcaatcatt

SEQ ID NO: 7 - DNA, 26 bp, Artificial sequence
  Primer KPNA2
taaggcagattttaagacacaaaagg

SEQ ID NO: 8 - DNA, 25 bp, Artificial sequence
  Primer KPNA2
gttcaactgttccaccactggtata

SEQ ID NO: 9 - DNA, 17 bp, Artificial sequence
  Primer CCNB1
catggcgctccgagtca

SEQ ID NO: 10 - DNA, 18 bp, Artificial sequence
  Primer CCNB1
gcgcctgccatgttgatc

SEQ ID NO: 11 - DNA, 16 bp, Artificial sequence
  Primer CDC2
gccgccgcggaataat

SEQ ID NO: 12 - DNA, 26 bp, Artificial sequence
  Primer CDC2
ccttctccaattttctctattttggt

SEQ ID NO: 13 - DNA, 20 bp, Artificial sequence
  Primer CDC20
cttccctgccagaccgtatc

SEQ ID NO: 14 - DNA, 24 bp, Artificial sequence
  Primer CDC20
ccaatccacaaggttcaggtaata

SEQ ID NO: 15 - DNA, 20 bp, Artificial sequence
  Primer MCM2
ggccacaacgtcttcaagga

SEQ ID NO: 16 - DNA, 23 bp, Artificial sequence
  Primer MCM2
atagttcaccaccaggctctcac

SEQ ID NO: 17 - DNA, 20 bp, Artificial sequence
  Primer GUSB
gagtggtgctgaggattggc

SEQ ID NO: 18 - DNA, 20 bp, Artificial sequence
  Primer GUSB
tctagcgtgtcgaccccatt

SEQ ID NO: 19 - DNA, 18 bp, Artificial sequence
  Primer TBP
gcccgaaacgccgaatat

SEQ ID NO: 20 - DNA, 23 bp, Artificial sequence
  Primer TBP
tcgtggctctcttatcctcatga

SEQ ID NO: 21 - DNA, 21 bp, Artificial sequence
  Primer RPLP0
accaaggaggacctcactgag

SEQ ID NO: 22 - DNA, 17 bp, Artificial sequence
  Primer RPLP0
accagcacgggcagcag

SEQ ID NO: 23 - DNA, 20 bp, Artificial sequence
  Primer TFRC
ggagccaggagaggacttcc

SEQ ID NO: 24 - DNA, 24 bp, Artificial sequence
  Primer TFRC
ttctccgacaactttctcttcagg

(19) United States
(12) Patent Application Publication
(10) Pub. No.: US 2011/0306507 A1
(43) Pub. Date: Dec. 15, 2011

(54) METHOD AND TOOLS FOR PROGNOSIS OF CANCER IN HER2+PARTIENTS

(75) Inventors: Christos Sotiriou, Bruxelles (BE); Benjamin Haibe-Kains, Bruxelles (BE); Christine Desmedt, Meise (BE)

(73) Assignee: Universite Libre de Bruxelles, Bruxelles (BE)

(21) Appl. No.: 12/733,575

(22) PCT Filed: Sep. 5, 2008
(86) PCT No.: PCT/EP2008/061827

§ 371 Date: Mar. 8, 2010

Related U.S. Application Data
(60) Provisional application No. 60/970,782, filed on Sep. 7, 2007.

(51) Int. Cl. C40B 30/04; C40B 40/10; C07K 14/47; C07K 16/40; C07K 16/18; C40B 60/12; C12N 9/64; C40B 40/08; C07H 21/00

(52) U.S. Cl. 506/9; 506/17; 506/18; 530/350; 536/23.5; 530/389.7; 506/39; 435/226; 536/23.2

(57) ABSTRACT

A gene or protein set includes at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, and possibly 40, 45, 50, 55, 60, 65 genes or proteins, antibodies or hypervariable portion thereof directed against the proteins encoded by these genes.

### FIELD OF THE INVENTION

The present invention is related to methods and tools for obtaining an efficient prognosis (prognostic) of cancer HER2+ patients wherein tumor invasion related genes are the keys player of breast cancer prognosis.

### BACKGROUND OF THE INVENTION

Breast cancer and especially invasive ductal carcinoma is the most common cancer in women in Western countries. Several prognostic signatures based on genetic profiling have been established. These different signatures all reflect the capacity of the tumor cells to proliferate¹. Their use permit to distinguish tumors with low and high proliferative activity, respectively the luminal A tumors characterized by a low proliferation rate and associated with good prognosis (prognostic) and a second group comprising the basal-like, HER2 (ERBB2) and luminal B tumors with high proliferation rate and associated with bad prognosis (prognostic).

Several studies have been realized about the role of the adaptive immune response in controlling the growth and recurrence of human tumors. In human colorectal cancer, it was shown that in situ analysis of tumor-infiltrating immune cells may be a valuable prognostic tool². Bates and al. showed that quantification of FOXP3-positive TR in breast tumors is valuable for assessing disease prognosis (prognostic) and progression³. Therefore, it exist a need to investigate biological processes that trigger breast cancer progression and that depend on a specific molecular subtype and a need to investigate the immune cells in breast cancer using human breast cancer model, especially CD4+ cells which regulate the immune response.

CD4+ cells belong to the leukocyte family which is a major component of the breast tumor microenvironment. CD4 marker is mainly expressed on helper T cells and with a limited level on monocyte/macrophages and dendritic cells. Immune cells play a role in tumor growth and spread, notably in breast tumor, and CD4+ cells are key players in the regulation of immune response.

Furthermore it is known that prognosis (prognostic) and management of breast cancer has always been influenced by the classic variables such as histological type and grade, tumor size, lymph node involvement, and the status of hormonal-estrogen (ER; ESR1) and progesterone receptors- and HER-2 (ERBB2) receptors of the tumor. Recently, different research groups identified several gene expression signatures predicting clinical outcome. A common feature to all these gene expression signatures is that they outperform conventional clinico-pathological criteria mostly by identifying a higher proportion of low-risk patients not necessarily needing additional systemic adjuvant treatment, while still correctly identifying the high-risk patients. Although they are all addressing the same clinical question, it might be surprising that there is only little or none overlap between the different gene lists, raising the question about their biological meaning. Also, although it has repeatedly and consistently been demonstrated that breast cancer, in addition to being a clinically heterogeneous disease, is also molecularly heterogeneous, with subgroups primarily defined by ER (ESR1), HER-2 (ERBB2) expression, the different prognostic signatures were never clearly evaluated and compared in these different molecular subgroups. This was probably due to the relatively small sizes of the individual studies, which would have made these findings statistically unstable.

Epithelial-stromal interactions are known to be important in normal mammary gland development and to play a role in breast carcinogenesis. Therefore, there exists a need to explore the influence of breast tumor microenvironment on primary tumor growth, breast cancer sub-typing and metastasis.

Therefore, it exists especially a need to investigate the biological processes and tumor markers that are involved in specific molecular subtype that do not belong to the status of the hormonal-estrogen (ER; ESR1) receptor, especially to investigate the biological process and tumor marker that are involved in the HER-2 (ERBB2) receptor molecular subtype.

### AIMS OF THE INVENTION

The present invention aims to provide methods and tools that could be used for improving the diagnosis (diagnostic) especially the prognosis (prognostic) of tumors, preferably breast tumors, especially in patient identified as HER2+/ERBB2 patients, in addition to the identification of patients identified as ER+ (ESR1+ patients) and/or ER− patients wherein immune response is the key player for cancer prognosis.

The present invention aims to provide methods and tools which improved the prognosis (prognostic) of patient and do not present drawbacks of the state of the art but also are able to propose a prognostic of all patients presenting a predisposition to tumors especially breast tumors development, which means patients which are identified as HER2+/ERBB2 patients, but also ER+ patients and ER-patients.

### SUMMARY OF THE INVENTION

The present invention is related to gene/protein set (or library) that is selected from mammal (preferably human) tumor invasion associated (or related) genes and proteins which are used for the prognosis (prognostic, detection, staging, predicting, occurrence, stage of aggressiveness, monitoring, prediction and possibly prevention) of cancer in HER2+ patients.

A first aspect of the present invention is related to a gene or protein set comprising or consisting of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 and possibly 40, 45, 50, 55, 60, 65 genes or proteins or the entire (gene) set selected from the table 12 and/or table 13 and (preferably monoclonal) antibodies (or hypervariable portion thereof) specifically directed against their encoded proteins sequences.

Advantageously, the gene and protein set according to the invention were selected from the gene and protein (including antibodies or their hypervariable portion thereof) that are bound to a solid support surface preferably according to an array.

The present invention is also related to a diagnostic kit or device comprising the gene or protein set according to the invention possibly fixed upon a solid support surface according to an array and possibly other means for real time PCR analysis (by suitable primers which allows a specific amplification of 1 or more of these genes selected from the gene set) or protein analysis.

The solid support could be selected from the group consisting of nylon membrane, nitrocellulose membrane, polyvinylidene difluoride, glass slide, glass beads, polyustyrene plates, membranes on glass support, CD or DVD surface, silicon chip or gold chip.

Preferably, set means for real time PCR analysis are means for qRT-PCR of the genes of the gene set (especially expression analysis (over or under expression) of these genes).

Another aspect of the present invention is related to a micro-array comprising one or more genes or proteins selected from the gene or protein set according to the invention, possibly combined with other genes or proteins selected from other genes or proteins sets for an efficient diagnosis (diagnostic) preferably prognosis (prognostic) of tumors, preferably breast tumors.

Another aspect of the present invention is related to a kit or device which is preferably a computerized system, comprising

a bio assay module configured for detecting gene expression (or protein synthesis) from a tumor sample, preferably based upon the gene or protein set according to the invention and

a processor module configured to calculate expression (over or under expression) of these genes (or synthesis of corresponding encoded proteins) and to generate a risk assessment for the tumor sample (risk assessment to develop a malignant tumor).

Preferably, the tumor sample is any type of tissue or cell sample obtained from a subject presenting a predisposition or a susceptibility to a tumor, preferably a breast tumor, that could be collected (extracted) from the subject. The subject could be any mammal subject, preferably a human patient and the sample could be obtained from tissues which are selected from the group consisting of breast cancer, colon cancer, lung cancer, prostate cancer, hepatocellular cancer, gastric cancer, pancreatic cancer, cervical cancer, ovarian cancer, liver cancer, bladder cancer, cancer of the urinary track, thyroid cancer, renal cancer, carcinoma, melanoma or brain cancer preferably, the tumor sample is a breast tumor sample.

Advantageously, the gene or protein set according to the invention could be combined, preferably in a diagnostic kit or device with other genes or proteins selected from other gene or protein sets preferably the gene or protein set(s) comprising or consisting of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95 and possibly 100, 105, 110 or the entire set selected from table 10 and/or table 11 or antibodies and hypervariable portion thereof directed against their encoded proteins for an efficient prognosis (prognostic) of other types of breast cancer (ER−, breast cancer type)(possibly combined with one or more gene of the set of genes as described by A. Teschendorff et al (genome biology nr 8,R157-2007 dedicated to efficient prognostic of cancer of ER− patient).

According to another embodiment of the present invention, the gene or protein set according to the invention comprises or consists of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95 genes or the entire set selected from the genes designated as upregulated genes in grade 3 tumors in the table 3 of the document WO 2006/119593 or antibodies directed against the corresponding encoded proteins. Preferably, these genes are proliferation related genes, preferably the gene set comprises at least the 8 genes selected from the group consisting of CCNB1, CCNA2, CDC2, CDC20, MCM2, MYBL2, KPNA2 and STK6.

Preferably, the selected genes/proteins are the 4 following genes/proteins: CCNB1, CDC2, CDC20 and MCM2 or more preferably CDC2, CDC20, MYBL2 and KPNA2 as described in the U.S. CIP patent application Ser. No. 11/929,043. These genes/proteins sequences are advantageously bound to a solid support as an array.

These genes/proteins present in a (diagnostic) kit or device may also further comprise means for real time PCR analysis of these preferred genes, preferably these means for real time PCR are means for qRT-PCR and comprise at least 8 sequences of the primers sequences SEQ ID NO 1 to SEQ ID NO 16.

Furthermore, these gene/protein sets may also further comprise reference genes/proteins, preferably 4 references genes for real time PCR analysis, which are preferably selected from the group consisting of the genes TFRC, GUS, RPLPO and TBP.

These reference genes are identified by specific primers sequences, preferably the primers sequences selected from the group consisting of SEQ ID NO 17 to SEQ ID NO 24.

With this set of genes, the person skilled in the art may also obtain (calculate) the gene expression grade index (GGI) or relapse score (RS).

The content of this previous PCT patent application (WO 2006/119593 and its CIP application Ser. No. 11/929,043 are incorporated herein by reference.

The person skilled in the art may also select other prognostic means (signatures) or gene/protein lists (gene/protein set which could be used for an efficient prognosis (prognostic) of cancer in ER− and ER+ patients such as the one described by:
Wang et al (lancet 365 (9460) p. 671-679 (2005)),
Van't Veer et al (Nature 415 (6871) p. 530-536 (2002)),
Paik et al (Engl. J. Med., 351 (27) p. 2817-2826 (2004)),
Teschendorff (Genome Biol., 7 (10) R101 (2006)),
Van De Vijver et al (Engl. J. Med. 347 (25) p. 1999-2009 (2002)),
Perou et al (Nature, 406, p 747-752 (2000))
Sotiriou et al, (PNAS 100 (18) p. 8414-8423 (2003)).
Sorlie et al (STNO—The Stanford/Norway dataset PNAS, 98 (19) p. 10869-10874 (2001)).
   http://genome-www.stanford.edu/breast.cancer/mopo.clinical/data.shtml and the expression profiling proteins used in breast cancer prognosis as described in the document WO 2005/071419 which comprises at least one, two, three or more genes or proteins selected from the group consisting of Afadin, Aurora A, a-Catenin, b-Catenin, BCL2, Cyclin D1, Cyclin E, Cytokeratin 5/6, Cytokeratin 8/18, E-Cadherin, EGFR, ERBB2, ERBB3, ERBB4, Estrogen receptor, FGFR1, FHIT, GATA3, Ki67, Mucin 1, P53, P-Cadherin, Progesterone receptor, TACC1, TACC2, TACC3 and possibly one or more gene or protein selected from the group consisting of Cytokeratin 6, Cytokeratin 18, Ang1, AuroraB, BCRP1, CathepsinD, CD10, CD44, CK14, Cox2, FGF2, GATA4, Hif1a, MMP9, MTA1, NM23, NRG1a, NRG1beta, P27, Parkin, PLAU, 5100, SCRIBBLE, Smooth Muscle Actin, THBS1, TIMP1.

The person skilled in the art may also select one or more gene used for analysis differential gene expression associated with breast tumor as described in the document WO 2005/021788 especially the sequence of the gene ERBB2, GATA4, CDH15, GRB7, NR1D1, LTA, MAP2, K6, PKM1, PPARBP, PPP1R1B, RPL19, PSB3, LOC148696, NOL3, loc283849, ITGA2B, NFKBIE, PADI2, STAT3, OAS2, CDKL5, STAITGB3, MKI67, PBEF, FADS2, LOX, ITGA2, ESTA1878915/NA, JDPA, NATA, CELSR2, ESTN33243/NA, SCUBE2, ESTH29301/NA, FLJ10193, ESRA and other gene or protein sequence described in the gene set of this PCT patent application.

The kit or device according to the invention may therefore comprise 1, 2, 3 or more gene/protein sets preferably dedicated to each type of patient group (ER-patient group, ER2+ patient group and HER2+ patient group) and could be included in a system which is a computerized system comprising 1, 2 or 3 bio assay modules configured for gene expression (or protein synthesis) of 1 or more of these gene/protein sets for an efficient diagnosis (prognosis) of all types (ER+, ER−, HER2+) of breast cancer. This system advantageously comprises one or more of the selected gene sets of the invention and a processor module configured to calculate a gene expression of this gene set(s) preferably a gene expression grade index (GGI) to generate a risk assessment for a selected tumor sample submitted to a diagnosis (diagnostic).

Advantageously, the molecules of the gene and protein set according to the invention are (directly or indirectly) labelled. Preferably, the label selected from the group consisting of radioactive, colorimetric, enzymatic, bioluminescent, chemoluminescent or fluorescent label for performing a detection, preferably by immunohistochemistry (IHC)analysis or any other methods well known by the person skilled in the art.

The present invention is also related to a method for the prognosis (prognostic) of cancer in a mammal subject preferably in a human patient preferably in at least ER− patient which comprises the step of collecting a tumor sample (preferably a breast tumor sample) from the mammal subject (preferably from the human patient) and measuring gene expression in the tumor sample by putting into contact sequences (especially mRNA sequences) with the gene/protein set according to the invention or the kit or device according to the invention and possibly generating a risk assessment for this tumor sample (preferably by designated the tumor sample as different subtypes within the ER− type and possibly in the ER+ and HER2+ types as being as higher risk and requiring a patient treatment regimen (for example adjusted to a specific chemotherapy treatment or specifically molecular targeted anti cancer therapy (such as immunotherapy or hormonotherapy).

In particular, the invention is also useful for selecting appropriate doses and/or schedule of chemotherapeutics and/or (bio)pharmaceuticals, and/or targeted agents, among which one may cite Aromatase Inhibitors, Anti-estrogens, Taxanes, Antracyclines, CHOP or other drugs like Velcade™, 5-Fluorouracil, Vinblastine, Gemcitabine, Methotrexate, Goserelin, Irinotecan, Thiotepa, Topotecan or Toremifene, anti-EGFR, anti-HER2/neu, anti-VEGF, RTK inhibitor, anti-VEGFR, GRH, anti-EGFR/VEGF, HER2/neu & EGF-R or anti-HER2.

Another aspect of the present invention is related to a method for controlling the efficiency of a treated method or an active compound in cancer therapy. Indeed, the method and tools according to the invention that are applied for an efficient prognosis of cancer in various breast cancer patient types, could be also used for an efficient monitoring of treatment applied to the mammal subject (human patient) suffering from this cancer.

Therefore, another aspect of the present invention is related to a method which comprises the prognosis (prognostic) method according to the invention before (and after) treatment of a mammal subject (human patient) with an efficient compound used in the treatment of subjects (patients) suffering from the diagnosis breast tumor. This means that this method requires a (first) prognosis (prognostic) step which is applied to the patient, before submitting said subject (patient) to a treatment and a (second) diagnosis (diagnostic) step following this treatment.

The inventors use CD10 and/or PLAU signatures according to Tables 12 and/or 13 as diagnosis and/or to assist the choice of suitable medicine.

This method could be applied several times to the mammal subject (human patient) during the treatment or during the monitoring of the treatment several weeks or months after the end of the treatment to reveal if a modification of genes expressions (or proteins synthesis) in a sample subject is obtained following the treatment.

Therefore, another aspect of the present invention is related to a method for a screening of compounds used for their anti tumoral activities upon tumors especially breast tumor, wherein a sufficient amount of the compound(s) is administrated to a mammal subject (preferably a human patient) suffering from cancer and wherein the prognosis (prognostic) method according to the invention is applied to said mammal subject before an administration of said active compound(s) and is applied following administration of said active compound(s) to identify, if the active compound(s) may modify the genetic profile (gene expression or protein synthesis) of the mammal subject.

A modification in the subject (patient) genetic profile (gene expression or protein synthesis) means that the obtained tumor sample before or after administration of the active compound(s) has been modified and will result into a different gene expression (or protein synthesis) in the sample (that is detectable by the gene set according to the invention). Therefore, this method is applied to identify if the active compound is efficient in the treatment of said tumor, especially breast tumor in a mammal subject, especially in a human patient.

Advantageously, in this method the active compound(s) which are submitted to this testing or screening method is recovered and is applied for an efficient treatment of mammal subject (human patient).

### DETAILED DESCRIPTION OF THE INVENTION

### In Vivo Interactions Between Breast Cancer (BC) Cells and Their Stromal Component/Analysis of Alterations in Gene Expressions.

The inventors have adapted the protocol described by Allinen and colleagues (2004) for the isolation of stroma cells and have managed to separate and isolate four different cell subpopulations: tumor epithelial cells (EpCAM positive), leukocytes (CD45 positive), myofibroblasts (CD10 positive) and endothelial cells. The inventors have also tested several RNAs amplification/labeling protocols for our gene expression experiments.

Up today, (myo)fibroblast cells (CD10) were isolated and purified from 28 breast tumors and 4 normal tissues. Gene expression analysis was performed using the Affymetrix GeneChip® Human Genome U133 Plus 2.0 arrays. Survival analysis was carried out using 12 publicly available micro-array datasets including more than 1200 systemically untreated breast cancer patients.

Breast tumor (myo)fibroblast stroma cells showed an altered gene expression patterns to the ones isolated from normal breast tissues (see Tables 12 and 13). While some of the differentially expressed genes are found to be associated with extracellular matrix formation/degradation and angiogenesis, the function of several other genes remains largely unknown.

Unsupervised hierarchical clustering analysis clustered breast tumor (myo)fibroblast cells into four main subgroups recapitulating the molecular portraits of breast cancer based on ER, HER2 status and tumor differentiation.

Similarly to tumor expression profiling studies, BC (myo)fibroblast cells isolated form intermediate grade tumors did not show a distinct gene expression pattern but a mixture of gene expression profiles similar to those derived from well and poorly differentiated tumors respectively.

A stroma gene expression signature developed from (myo)fibroblast cells isolated from normal versus BC tissues showed a statistically significant association with clinical outcome. Breast tumors with high expression levels of the stroma signature were significantly associated with worse prognosis (HR 1.55; CI 1.20-1.99; p=5.57 10⁻⁴). This association was mainly observed within the clinically high risk HER2+ subtypes. Interestingly, HER2+ tumors with high and low expression levels of the stroma signature showed 45% and 85% distant metastasis free survival at 5-year follow-up respectively (HR 2.53; CI 1.31-4.90; p=5.29 10⁻³).

Preliminary results highlight the importance of tumor epithelial-stroma cell interactions in breast carcinogenesis and breast cancer sub-typing. Moreover, it shows the role of stroma cells in tumor dissemination particularly within the HER2+ subtype and provide basis for the development of novel therapeutic strategies.

### Investigation of the Tumor Invasion and Immune Response Using in Silico Data

### Material and Methods

### Gene Expression Data

Gene expression datasets were retrieved from public databases or authors' website. The inventors have used normalized data (log 2 intensity in single-channel platforms or log 2 ratio in dual-channel platforms) as published by the original studies. No processing of gene expression data was necessary because of the meta-analytical framework of this study.

### Probe Annotation and Mapping

Hybridization probes were mapped to Entrez GeneID [19] through sequence alignment against RefSeq mRNA in the (NM) subset, similar to the approach by Shi et al.[20], using RefSeq version 21 (2007.01.21) and Entrez database version 2007.01.21. When multiple probes were mapped to the same GeneID, the one with the highest variance in a particular dataset was selected to represent the GeneID.

### Prototype-Based Co-Expression Modules

The inventors have considered a set of prototypes, i.e. genes known to be related to specific biological processes in breast cancer (BC) and aimed to identify the genes that are specifically co-expressed with each of them. To this end, the inventors computed for each gene the direct and the combined associations. The direct association is defined as the linear correlation between gene i and each prototype j separately, whereas the combined association is defined as the linear correlation between gene i and the best linear combination of prototypes, as identified by feature selection (orthogonal Gram-Schmidt feature selection [21]). Considering all the direct and combined associations obtained for gene i, a Friedman's test was used in order to identify the significantly highest associations. In case only one direct association (with prototype j) was left over, then gene i was assigned to module j and was noted as “specific” to prototype j. In contrast, if the highest associations included the multivariate association or several direct associations, then gene i was not assigned to any module j and was noted as “related” to all prototypes involved in the highest associations. A threshold on correlation allowed us to discard the genes that were not correlated to any prototypes. This method was applied in a meta-analytical framework, combining results from NKI2 (4) and VDX (16) datasets (581 patients, see Table 1). Table 1 represents characteristics of the publicly available gene expression datasets. Note that some samples are used in several studies. The following study ids have samples in common: NKI/NKI2 and UPP/STK/UNT/TBAGD/TBVDX/TAM. For all analyses, the inventors removed duplicated patients from small datasets (e.g. NKI) to avoid decreasing the sample size of large datasets (e.g. NKI2).

TABLE 1

| | | Number of patients | | Gene |
|---|---|---|---|---|
| Data- | | (% of untreated | | expression |
| set | Id | patients) | | platform |
| NKI | NKI | 117 | (95.8%) | Agilent |
| NKI | NKI2 | 295 | (55.9%) | Agilent |
| | | | | Stanford |
| STNO2 | STNO2 | 122 | (18%) | Microarray |
| | | | | cDNA National |
| NCI | NCI | 99 | (11.1%) | Cancer Institute |
| MGH | MGH | 60 | (0%) | Arcturus |
| UPP | UPP | 251 | (68.1%) | Affymetrix |
| STK | STK | 159 | (unknown) | Affymetrix |
| VDX | VDX | 286 | (100%) | Affymetrix |
| VDX2 | VDX2 | 180 | (100%) | Affymetrix |
| UNT | UNT | 137 | (100%) | Affymetrix |
| UNC | UNC | 153 | (0%) | Affymetrix |
| TRANSBIG | TBAGD | 307 | (100%) | Affymetrix |
| TRANSBIG | TBVDX | 198 | (100%) | Affymetrix |
| TAM | TAM | 255 | (0%) | Affymetrix |

The whole procedure is sketched in Supplementary FIG. 1. In order to identify genes that are coexpressed with one specific prototype, the inventors used a database of 581 patients from NKI2 and VDX datasets. First, they considered only the intersection of genes between the Affymetrix and Agilent platforms after having applied the mapping procedure as described above (see Section Probe annotation and mapping). The inventors refer hereafter to NKI2 and VDX reduced datasets as gene expressions of this intersection. The following procedure, sketched in Supplementary FIG. 1, is performed for each gene of the NKI2 and VDX reduced datasets:

1 All univariate linear models were fitted using prototypes as explanatory variable and the gene i as response variable in the NKI2 and VDX reduced datasets, resulting in seven couples of univariate linear models.
2 To test whether variability in coefficient estimates between the two platforms are due to sampling error alone, the inventors applied a stringent test of heterogeneity [Cochrane, 1954; 25] for each couple of coefficients. If at least one coefficients is heterogeneous (p-value<0.01), gene i was discarded for further analysis.
3 The inventors compared a set of linear models to identify if gene i is predictable by only one prototype, i.e. one model is significantly better than all the other candidates. To do so, we used the PRESS statistic [Allen, 1974; ref 22] to compute efficiently the leave-one-out cross-validation (LOOCV) errors and compared two models on the basis of their vector of LOOCV errors. A Friedman's test was used to identify the set of best models for NKI2 and VDX reduced datasets separately. For each comparison, the two p-values were meta-analytically combined using the Z-transform method [Whitlock, 2005]. A model was considered as significantly better than another one if the combined p-value<0.05. Because of computational limitation, we were not able to test all possible combinations of prototypes to predict gene i. Only the best set of prototypes with respect to mean squared LOOCV error of the corresponding multivariate linear model was identified using the orthogonal Gram-Schmidt feature selection [Chen et al., 1989]; ref 21. This multivariate model was used in addition to the set of univariate models.
4 The inventors tested the specificity of gene to one prototype by looking at this set of best models. If only one univariate model belonged to this set, it meant that the model using only the prototype j was significantly better than all the models with the other prototypes. Additionally, if the multivariate model belonged to the set of best models, it meant that the multivariate model is not significantly better than the model with prototype j.
5 Gene i was identified to be specific to prototype j and was included in the module, also called gene list, j.
In order to reduce the size of the modules, we filtered the specific genes using a threshold of 0.95 on the normalized mean squared LOOCV error.

### Module Scores

For a specific dataset, the module score was computed for each sample as:

$$Module score = {\sum }_{i} WiXi {\sum }_{i} \vert Wj \vert$$

where xᵢ is the expression of a gene in the module that is present in the dataset's platform. wᵢ is either +1 or −1 depending on the sign of the association with the prototypes. Robust scaling was performed on each module score to have the interquartile range equals to 1 and the median equals to 0 within each dataset, allowing for comparison between module scores.

### Gene Ontology and Functional Analysis

Gene ontology analyses were executed using Ingenuity Pathways Analysis tools (Ingenuity Systems, Mountain View, Calif. www.ingenuity.com), a web-delivered application that enables the discovery, visualization, and exploration of molecular interaction networks in gene expression data. The lists of genes identified to be specifically associated with the different prototypes, containing the HUGO gene symbol as well as an indication of positive or negative co-expression, were uploaded into the Ingenuity pathway analysis and correlated with the functional annotations stored in the Ingenuity pathway knowledge base.

### Clustering

In order to consistently identify molecular subgroups across the different datasets, we clustered the tumors using the ER (ESR1) and HER2(ERBB2) module scores by fitting Gaussian mixture models [23] with equal and diagonal variance for all clusters. The inventors have used the Bayesian Information Criterion [24] to test the number of components. Each tumor was automatically classified to one of the identified molecular subgroups using the maximum posterior probability of membership in the clusters.

### Association Analysis

The inventors have estimated the pairwise correlation of the module scores using Pearson's correlation coefficient. Each correlation coefficient was estimated for each dataset separately and combined with inverse variance-weighted method with fixed effect model [25]. Additionally, the inventors have tested the association between module scores and subtypes using Kruskal-Wallis test. The inventors have tested the association between module scores and clinical variables using Wilcoxon rank sum test. Each statistical test was applied for each dataset separately and p-values were combined using the inverse normal method with fixed effect model [29]. These association analyses were carried out both in the global population and in the different molecular subgroups.

### Survival Analysis

The inventors have considered the relapse-free survival (RFS) of untreated patients as the survival endpoint. When RFS was not available, the inventors have used distant metastasis free survival (DMFS) data. All the survival data were censored at 10 years. Survival curves were based on Kaplan-Meier estimates, with the Greenwood method for computing the 95% confidence intervals. Hazard ratios between two or three groups (subtypes and ternary module scores) were calculated using Cox regression with the dataset as stratum indicator, thus allowing for different baseline hazard functions between cohorts. For clinical variables and module scores, the hazard ratios were estimated for each dataset separately and combined with inverse variance-weighted method with fixed effect model [25]. The inventors have used a forward stepwise feature selection in a meta-analytical framework to identify the best multivariable Cox models. The significance thresholds regarding the combined p-values (Wald test for hazard ratio) for the inclusion of a new feature (variable) and for the exclusion of a previously selected feature (variable) were set to 0.05.

### Application of the Prognostic Gene Signatures

When cross-platform mapping was necessary, the inventors have only considered genes in the signatures that could be mapped to GeneID. A prediction score was computed for each signature, using a linear combination similar to the formula for module score above. Gene-specific weights (coefficients, correlations, or other measures) from the original studies were converted in +1 or −1 depending on the original up- or down-regulation of each gene. This computation method for previously published gene classifiers gave very similar results compared to the official classifications on the original datasets and allowed the application of gene signatures on different micro-array platforms. Robust scaling was performed on each gene signature to have the interquartile range equals to 1 and the median equals to 0 within each dataset, to allow for comparison between the different gene signatures.

### Results

### FIGURE LEGEND

FIG. 1 represents joint distribution between the ER (ESR1) and HER2(ERBB2) module scores for three example datasets: NKI2 (A), UNC (B), VDX (C). Clusters are identified by Gaussian mixture models with three components. The ellipses shown are the multivariate analogs of the standard deviations of the Gaussian of each cluster.

FIG. 2 represents survival curves for untreated patients stratified by molecular subtypes ESR1−/ERBB2−, ERBB2+ and ESR1+/ERBB2−.

FIG. 3 represents forest plots showing the log 2 hazard ratios (and 95% CI) of the univariate survival analyses in the global population (A) and in the ESR1−/ERBB2− (B), the ERBB2+ (C) and in the ESR1+/ERBB2− (D) subgroups of untreated breast cancer patients.

FIG. 4 represents Kaplan-Meier curves of the module scores which were significant in the univariate analysis in the molecular subgroup analysis. The module scores were split according to their 33% and 66% quantiles. STAT1 module in the ESR1−/ERBB2− subgroup (A), PLAU module in the ERBB2+ subgroup (B), STAT1 module in the ERBB2+ module (C), AURKA module in the ESR1+/ERBB2− subgroup (D).

FIG. 5 shows the Kaplan-meier survival curves for the ERB2+ subgroup of patients having low, intermediate and high scores for the combination of the tumor invasion and immune module scores.

FIG. 6 sketches the method used to identify prototype-based co-expression modules.

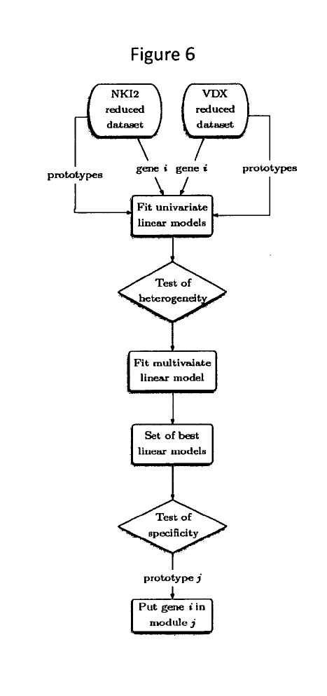

### DEFINING THE MOLECULAR MODULES OF BREAST CANCER

To develop the molecular modules the inventors have first selected typical genes to act as “prototypes” for each biological process, based on the literature and then applied a comparison of linear models (see methods) to generate modules of genes specifically associated with each of the prototype genes underlying different biological processes in breast cancer. The selected prototype genes were: AURKA (also known as STK6, 7 or 15), PLAU (also known as uPA), STAT1, VEGF, CASP3, ER (ESR1) and HER2(ERBB2), representing the proliferation, tumor invasion/metastasis, immune response, angiogenesis, apoptosis phenotypes and the ER (ESR1) and HER2 signaling respectively.

To identify genes that would perform well across multiple micro-array platforms and different breast cancer populations, the inventors have defined these molecular modules by analyzing a database of 581 breast tumors samples included in the van de Vijver et al. [4], and Wang et al. series [16], hybridized on Agilent and Affymetrix arrays respectively. Each module score was defined by the difference of the sums of the positively and negatively correlated genes for the chosen prototype only. In case a gene was correlated with more than one prototype, then it was not included in any module. These lists of genes are available as Table 2, see below. The inventors then mapped and computed each of these module scores on several published micro-array datasets totaling over 2100-tumor samples (see Table 1).

The main characteristics of these molecular modules are that they are identified as genes that are co-expressed consistently with the chosen prototypes in datasets using Agilent and Affymetrix micro-array platforms and that they are identified without looking at clinical variables and gene annotation.

### Characterization of the Genes Included in the Molecular Modules

The seven lists of genes representing the molecular modules, along with their sign, were uploaded into the Ingenuity pathway knowledge database (IPKB) for analysis of functional annotations.

The ER (ESR1) module was composed of 469 genes and as expected characterized by the co-expression of several luminal and basal genes already reported by previous micro-array studies such as XBP1, TFF1, TFF3, MYB, GATA3, PGR and several keratins. Information was found in the IPKB for 326 of these genes and 139 were significantly associated with a particular function such as small molecule biochemistry, cancer-related functions, lipid metabolism, cellular movement, cellular growth and proliferation or cell death. The HER2(ERBB2) module included 28 genes, with nearly half of them co-located on the 17q11-22 amplicon, such as THRA, ITGA3 and PNMT. Sixteen could be used for functional analysis and 15 were significantly associated with the following ontology classes: cancer-related functions, cell-to-cell signaling, cellular growth and proliferation, molecular transport and cell morphology. The proliferation module (AURKA) included 229 genes, with 34 of them represented in the previously reported genomic grade index. One hundred forty-three genes matched the IPKB, out of which 93 were significantly associated with a particular function. As expected, the majority of these genes, such as CCNB1, CCNB2, BIRC5, were involved in cellular growth and proliferation, cancer and cell cycle related functions. The tumor invasion/metastasis module (PLAU) included 68 genes with several metalloproteinases among them. Out of the 55 that mapped the IPKB, 46 were significantly associated with functions such as cellular movement, tissue development, cellular development and cancer-related functions. The immune response module (STAT1) included 95 genes and the functional analysis carried out on 82 of them revealed that the majority was associated with immune response, followed by cellular growth and proliferation, cell-signaling and cell death. The angiogenesis module (VEGF) included 10 genes related with cancer, gene expression, lipid metabolism and small molecule biochemistry and finally the apoptosis module (CASP3) included 9 genes mainly associated with protein synthesis and degradation, as well as cellular assembly and movement.

It is worth noting that for all the prototypes the lists of genes related to each prototype were much longer than the ones presented here, which represent the genes specifically associated to a given prototype taking into account the correlation with the other prototypes (Table 3).

TABLE 3

| | Nr of genes associated | Nr of genes specifically associated | |
|---|---|---|---|
| Prototype | with the prototype* | with the prototype** | |
| ESR1 | 990 | 468 | (47%) |
| ERBB2 | 158 | 27 | (17%) |
| AURKA | 730 | 228 | (31%) |
| PLAU | 241 | 67 | (28%) |
| STAT1 | 480 | 94 | (20%) |
| VEGF | 307 | 13 | (4%) |
| CASP3 | 76 | 9 | (12%) |

Table 3 represents number of genes associated with each prototype.
*These numbers represent the number of genes related with a given prototype, i.e. these genes may also be associated with another prototype.
**These numbers represent the number of genes specifically associated with a given prototype, which means that these genes are only associated to this prototype and not to others.
For example, the expression of chemokine IL8, which has been reported to have pro-angiogenic effects, was indeed associated with the expression of VEGF. However, since its expression was also correlated with the expression of PLAU, it was not included in any module. The apoptosis-related genes BCL2A1, BIRC3, CD2 and CD69 were not integrated in the apoptosis module, as their expression was also associated with ER (ESR1). Also, additional metalloproteases were found to be associated with PLAU, such as MMP1 and MMP9, but as their expression levels were also correlated with ER (ESR1) and STAT1, they were not included in the invasion module. This shows that the different biological processes are most probably interconnected, but here the inventors wanted to make them “specific” in order to better depict their individual impact on breast cancer biology and prognosis (prognostic).

The expression values of the genes included in the different modules were summarized in module scores for further analysis (see the “module score” section in the methods for details regarding the computation).

### Identification and Characterization of the ESR1−/ERBB2−, ESR1+/ERBB2− and ERBB2+Molecular Subgroups

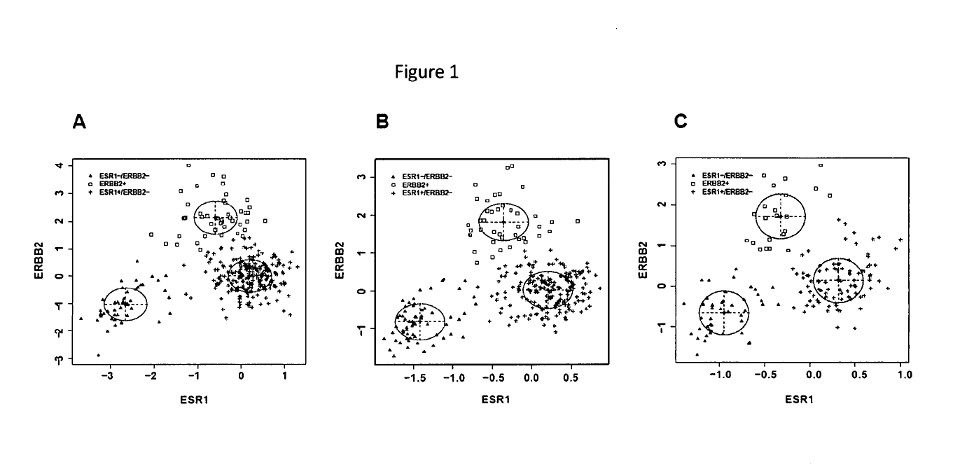

Since the inventors wanted to perform the analyses on the global population but also in the different subgroups based on the ER (ESR1) and HER2 modules, they needed to define these three molecular subgroups. To this end, the inventors used a clustering approach which consistently identified the three groups of patients in the different datasets, except for the MGH and VDX2/TBAGD datasets, due to the lack of ESR1− patients and the small number of probes respectively. The clusters for the NKI2, VDX and UNC cohorts are shown in FIG. 1 as an example.

The clinico-pathological characteristics per molecular subgroup are illustrated in Table 4.

TABLE 4

| | ESR1−/ERBB2− | | ERBB2+ | | ESR1+/ERBB2− | |
|---|---|---|---|---|---|---|
| Number of | subgroup | | subgroup | | subgroup | |
| patients (%) | (N = 189) | | (N = 129) | | (N = 628) | |
| Age | | | | | | |
| ≦50 years | 132 | (70) | 76 | (59) | 334 | (53) |
| >50 years | 57 | (30) | 53 | (41) | 294 | (47) |
| Size | | | | | | |
| ≦2 cm | 121 | (64) | 84 | (65) | 457 | (73) |
| >2 cm | 68 | (36) | 41 | (32) | 170 | (27) |
| Unknown | 0 | | 4 | (3) | 1 | (0) |
| Nodal status | | | | | | |
| Negative | 166 | (88) | 109 | (84) | 578 | (92) |
| Positive | 23 | (12) | 15 | (12) | 45 | (7) |
| Unknown | 0 | | 5 | (4) | 5 | (1) |
| Tumor grade | | | | | | |
| I | 5 | (3) | 3 | (2) | 131 | (21) |
| II | 19 | (10) | 31 | (24) | 238 | (38) |
| III | 151 | (80) | 70 | (54) | 189 | (30) |
| Unknown | 14 | (7) | 25 | (20) | 70 | (11) |
| Estrogen | | | | | | |
| receptors | | | | | | |
| Negative | 161 | (85) | 67 | (52) | 35 | (5) |
| Positive | 27 | (14) | 58 | (45) | 588 | (94) |
| Unknown | 1 | (1) | 4 | (3) | 5 | (1) |

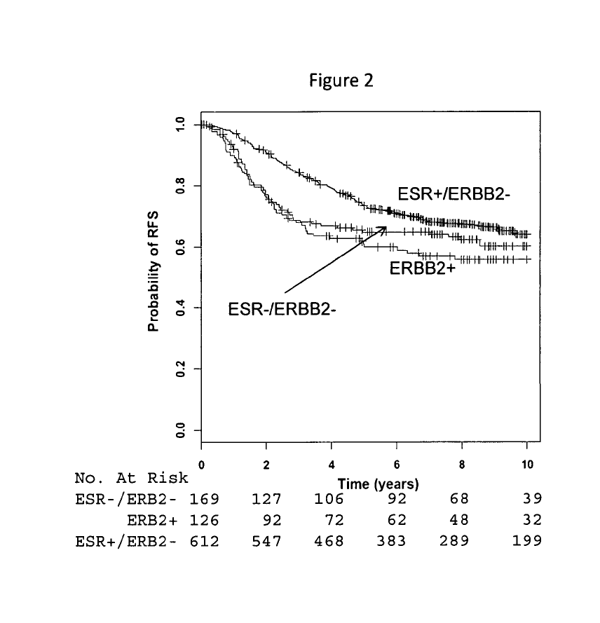

Table 4 represents clinico-pathological characteristics per molecular subgroup for the untreated breast cancer patients considered for the survival analyses. As one would expect, the vast majority of the tumors in the ESR1−/ERBB2− and ERSR1+/ERBB2− subgroups were negative and positive respectively for the ER (ESR1) protein status. On the contrary, the ERBB2+ subgroup was composed by a mixture of tumors with regard to the ER (ESR1) protein status. When comparing the survival curves of these three molecular subgroups across all the untreated patients of this meta-analysis, the inventors observed differences between the molecular subgroups, as already reported by others [27-31]. Indeed, the survival curve from the ESR1+/ERBB2− was significantly different from the two others (p=0.03 for ESR1−/ERBB2− and p=0.003 for ERBB2+). However, no difference in survival was noticed between the ESR1−/ERBB2− and ERBB2+ subgroups (p=0.56; see FIG. 2).

### Association Between Clinico-Pathological Parameters and Molecular Module Scores

Looking at the information on the 2180 patients, we started by investigating whether there was any association between the different module scores. One interesting finding was for example the positive and negative correlation between the proliferation module score on one hand and the angiogenesis and tumor invasion module scores on the other hand. These associations were conserved throughout the different molecular subtypes, with the highest correlations being observed in the ESR1−/ERBB2− subgroup. All results are provided in Table 5.

TABLE 5

| | | | | | |
|---|---|---|---|---|---|
| ERBB2 | AURKA | PLAU | VEGF | STAT1 | CASP3 |

(A) Global population

| | | | | | | |
|---|---|---|---|---|---|---|
| CASP3 | | | | | | |
| STAT1 | | | | | | 0.170 |
| VEGF | | | | | −0.290 | −0.108 |
| PLAU | | | | 0.009 | −0.007 | −0.134 |
| AURKA | | | −0.300 | 0.421 | 0.215 | 0.101 |
| ERBB2 | | 0.001 | 0.025 | 0.080 | −0.029 | −0.000 |
| ESR1 | 0.170 | −0.314 | −0.182 | −0.108 | −0.304 | −0.008 |

(B) ESR1−/ERBB2− subgroup

| | | | | | | |
|---|---|---|---|---|---|---|
| CASP3 | | | | | | |
| STAT1 | | | | | | 0.200 |
| VEGF | | | | | −0.410 | −0.265 |
| PLAU | | | | 0.009 | −0.134 | −0.158 |
| AURKA | | | −0.521 | 0.551 | 0.035 | 0.050 |
| ERBB2 | | −0.032 | 0.124 | 0.141 | −0.210 | −0.220 |
| ESR1 | 0.293 | −0.510 | 0.051 | −0.298 | −0.022 | −0.037 |

(C) ERBB2+ subgroup

| | | | | | | |
|---|---|---|---|---|---|---|
| CASP3 | | | | | | |
| STAT1 | | | | | | 0.070 |
| VEGF | | | | | −0.304 | −0.402 |
| PLAU | | | | 0.140 | 0.017 | −0.255 |
| AURKA | | | −0.201 | 0.400 | 0.144 | −0.050 |
| ERBB2 | | 0.179 | −0.145 | 0.105 | −0.150 | −0.012 |
| ESR1 | 0.165 | 0.075 | −0.214 | 0.005 | −0.287 | 0.006 |

(D) ESR1+/ERBB2− subgroup

| | | | | | | |
|---|---|---|---|---|---|---|
| CASP3 | | | | | | |
| STAT1 | | | | | | 0.174 |
| VEGF | | | | | −0.341 | −0.213 |
| PLAU | | | | −0.006 | 0.072 | −0.144 |
| AURKA | | | −0.360 | 0.245 | 0.170 | 0.112 |
| ERBB2 | | 0.271 | −0.087 | 0.171 | −0.045 | −0.103 |
| ESR1 | 0.050 | 0.171 | −0.306 | 0.262 | −0.318 | −0.161 |

Table 5 refers to the following four tables: meta-estimators of pair-wise Pearson's correlation coefficients between module scores of 2180 treated and untreated breast cancer patients from the global population (A), 319 patients from the ESR1−/ERBB2subgroup (B), 252 patients from the ERBB2+ subgroup (C) and 1610 patients from the ESR1+/ERBB2− subgroup (D).

The inventors further sought to characterize the association between the module scores and the well established clinico-pathological parameters such age, tumor size, nodal status, histological grade and ER (ESR1) status defined either by immunohistochemistry (IHC) or by ligand binding assay. Meaningful associations were found, establishing the validity of module scores. For instance, highly significant associations were observed between ESR1/proliferation module scores and ER (ESR1) protein status/histological grade. The inventors also noticed less known or new associations, such as for example a positive association between histological grade and the angiogenesis, immune response and apoptosis module values. The same associations were also reported for nodal involvement. However, the inventors did not observe any association between the invasion module values and the clinico-pathological markers. When investigating these associations in the different molecular subgroups, the inventors found similar associations in the ESR1+/ERBB2− subgroup, with one major difference being the highly significant correlation between the ERRBB2 module scores and the histological grade which was not observed in the global population. On the contrary, very few significant associations were reported in the two other subgroups. These results are summarized in Table 6.

TABLE 6

| | | | | |
|---|---|---|---|---|
| Age | Tumor Size | Nodal | ESR1-status | |
| (≦50 vs | (≦2 vs | Status | (IHC or LAB*, | Grade |
| >60 years) | >2 cm) | (− vs +) | − vs +) | (1 vs 3) |

(A) Global population

| | | | | | |
|---|---|---|---|---|---|
| ESR1 | + + + | − | − | + + + | − − − |
| ERBB2 | NS | + | + | + + + | NS |
| AURKA | NS | + + + | + + + | − − − | + + + |
| PLAU | NS | NS | NS | NS | NS |
| VEGF | NS | + + + | + + | − − − | + + + |
| STAT1 | − | NS | + + + | − − − | + + + |
| CASP3 | NS | + + | + + | − − | + + + |

(B) ESR1−/ERBB2− subgroup

| | | | | | |
|---|---|---|---|---|---|
| ESR1 | NS | NS | NS | NS | NS |
| ERBB2 | NS | NS | NS | NS | NS |
| AURKA | NS | + + | NS | − | + |
| PLAU | NS | NS | NS | NS | NS |
| VEGF | NS | NS | NS | NS | NS |
| STAT1 | NS | NS | NS | NS | NS |
| CASP3 | NS | NS | NS | −− | NS |

(C) ERBB2+ subgroup

| | | | | | |
|---|---|---|---|---|---|
| ESR1 | NS | NS | − | + + + | − |
| ERBB2 | NS | NS | NS | NS | NS |
| AURKA | NS | + + | NS | NS | NS |
| PLAU | NS | NS | NS | NS | NS |
| VEGF | + + | NS | NS | NS | NS |
| STAT1 | NS | NS | NS | NS | NS |
| CASP3 | NS | NS | NS | NS | NS |

(D) ESR1+/ERBB2− subgroup

| | | | | | |
|---|---|---|---|---|---|
| ESR1 | + + + | NS | NS | + + + | − − − |
| ERBB2 | NS | + | + + + | NS | + + + |
| AURKA | NS | + + + | + + + | − − − | + + + |
| PLAU | NS | NS | − | NS | NS |
| VEGF | NS | + + | + | + | + + + |
| STAT1 | − | NS | + + + | − − | + + + |
| CASP3 | NS | + + | + + | − | + + + |

Table 6 refers to the following four tables: association between the module scores and the clinico-pathological parameters for the global population (A), ESR1−/ERBB2(B), ERBB2+ (C) and ESR1+/ERBB2− (D) subgroups. The “+” sign represents a positive association between the variables with a p-value comprised between 0.01 and 0.05 (+), between 0.01 and 0.001 (++) and <0.001 (+++). The “−” sign represents a negative association between the variables with a p-value comprised between 0.01 and 0.05 (−), between 0.01 and 0.001 (−−)

### Molecular Modules, Clinico-Pathological Parameters and Prognosis (Prognostic)

To evaluate the prognostic value of these module scores in relation with the natural history of the disease the inventors considered only untreated breast cancer patients including 1235 tumor samples. For that purpose the inventors performed both, univariate and multivariate analysis for relapse free survival on systemically untreated patients with a mean follow-up of 7.4 years including well established clinico-pathological variables as well as the molecular modules defined in this study. These analyses were stratified according to the molecular subgroups to take into consideration the differences in survival over time of these three subgroups of patients (see FIG. 2).

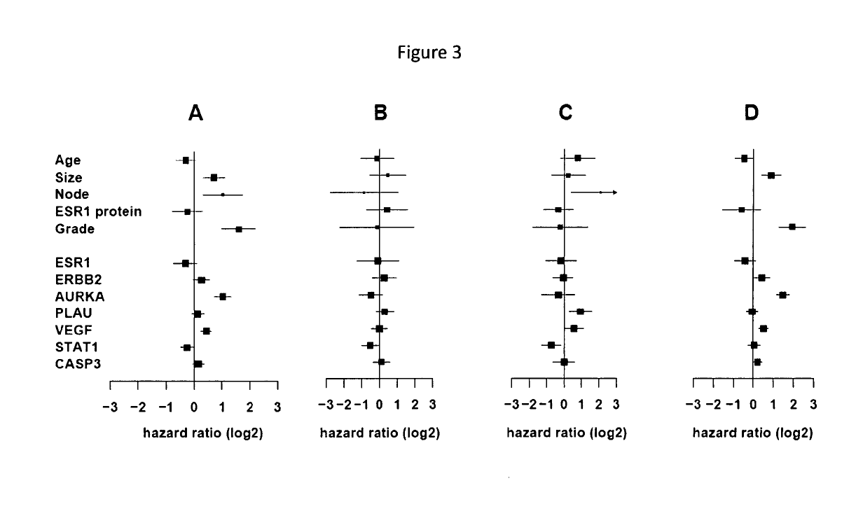

In a univariate model, almost all “well-established” clinico-pathological parameters, namely tumor size, histological grade, and nodal invasion, were significantly associated with clinical outcome. Among the molecular modules, proliferation, angiogenesis and immune response also displayed a statistically significant association with relapse free survival. Given the small percentage (6.7%, 83 out of 1225) of patients with nodal involvement, survival analysis results for nodal status should be interpreted with caution. The results of this univariate analysis are illustrated in FIG. 3 and shown in more details in Table 7.

TABLE 7
(A) Global population

| | hr | lower.95 | upper.95 | p | n |
|---|---|---|---|---|---|
| age | 0.813 | 0.630 | 1.050 | 1.13 10⁻⁰¹ | 876 |
| size | 1.641 | 1.248 | 2.157 | 3.90 10⁻⁰⁴ | 887 |
| node | 2.038 | 1.249 | 3.328 | 4.40 10⁻⁰³ | 315 |
| er | 0.844 | 0.581 | 1.228 | 3.75 10⁻⁰¹ | 888 |
| grade | 3.029 | 1.989 | 4.611 | 2.38 10⁻⁰⁷ | 802 |
| ESR1 | 0.801 | 0.601 | 1.068 | 1.31 10⁻⁰¹ | 907 |
| ERBB2 | 1.203 | 0.984 | 1.469 | 7.08 10⁻⁰² | 907 |
| AURKA | 2.040 | 1.666 | 2.497 | 4.84 10⁻¹² | 907 |
| PLAU | 1.095 | 0.939 | 1.277 | 2.47 10⁻⁰¹ | 907 |
| VEGF | 1.346 | 1.177 | 1.540 | 1.52 10⁻⁰⁵ | 907 |
| STAT1 | 0.845 | 0.715 | 0.998 | 4.78 10⁻⁰² | 907 |
| CASP3 | 1.117 | 0.973 | 1.281 | 1.15 10⁻⁰¹ | 907 |

(B) ESR1−/ERBB2− subgroup

| | hazard ratio | lower.95 | upper.95 | p-value | n |
|---|---|---|---|---|---|
| age | 0.918 | 0.485 | 1.737 | 7.92 10⁻⁰¹ | 133 |
| size | 1.388 | 0.687 | 2.804 | 3.61 10⁻⁰¹ | 82 |
| node | 0.549 | 0.149 | 2.020 | 3.67 10⁻⁰¹ | 37 |
| cr | 1.348 | 0.610 | 2.981 | 4.60 10⁻⁰¹ | 144 |
| grade | 0.903 | 0.212 | 3.851 | 8.90 10⁻⁰¹ | 89 |
| ESR1 | 0.938 | 0.411 | 2.138 | 8.78 10⁻⁰¹ | 165 |
| ERBB2 | 1.212 | 0.757 | 1.940 | 4.22 10⁻⁰¹ | 161 |
| AURKA | 0.721 | 0.458 | 1.135 | 1.57 10⁻⁰¹ | 169 |
| PLAU | 1.237 | 0.879 | 1.739 | 2.22 10⁻⁰¹ | 156 |
| VEGF | 1.001 | 0.737 | 1.360 | 9.93 10⁻⁰¹ | 165 |
| STAT1 | 0.698 | 0.496 | 0.982 | 3.92 10⁻⁰² | 169 |
| CASP3 | 1.082 | 0.771 | 1.519 | 6.47 10⁻⁰¹ | 165 |

(C) ERBB2+ subgroup

| | hazard ratio | lower.95 | upppr.95 | p-value | n |
|---|---|---|---|---|---|
| age | 1.709 | 0.862 | 3.387 | 1.25 10⁻⁰¹ | 108 |
| size | 1.171 | 0.594 | 2.307 | 6.48 10⁻⁰¹ | 76 |
| node | 4.318 | 1.314 | 14.192 | 1.60 10⁻⁰² | 29 |
| er | 0.795 | 0.436 | 1.450 | 4.54 10⁻⁰¹ | 107 |
| grade | 0.851 | 0.285 | 2.542 | 7.72 10⁻⁰¹ | 95 |
| ESR1 | 0.880 | 0.478 | 1.621 | 6.82 10⁻⁰¹ | 126 |
| ERBB2 | 0.963 | 0.650 | 1.427 | 8.50 10⁻⁰¹ | 126 |
| AURKA | 0.796 | 0.413 | 1.536 | 4.97 10⁻⁰¹ | 126 |
| PLAU | 1.914 | 1.214 | 3.018 | 5.22 10⁻⁰³ | 126 |
| VEGF | 1.483 | 1.003 | 2.195 | 4.86 10⁻⁰² | 126 |
| STAT1 | 0.595 | 0.403 | 0.878 | 8.99 10⁻⁰³ | 126 |
| CASP3 | 0.993 | 0.650 | 1.516 | 9.73 10⁻⁰¹ | 126 |

(D) ESR1+/ERBB2− subgroup

| | hazard ratio | lower.95 | upper.95 | p-value | n |
|---|---|---|---|---|---|
| age | 0.717 | 0.522 | 0.985 | 4.01 10⁻⁰² | 598 |
| size | 1.813 | 1.301 | 2.527 | 4.45 10⁻⁰⁴ | 605 |
| node | | | | | 233 |
| er | 0.658 | 0.340 | 1.273 | 2.14 10⁻⁰¹ | 515 |
| grade | 3.862 | 2.418 | 6.168 | 1.55 10⁻⁰⁸ | 538 |
| ESR1 | 0.751 | 0.525 | 1.073 | 1.15 10⁻⁰¹ | 605 |
| ERBB2 | 1.348 | 1.027 | 1.770 | 3.13 10⁻⁰² | 605 |
| AURKA | 2.784 | 2.219 | 3.493 | 9.03 10⁻¹⁹ | 598 |
| PLAU | 0.963 | 0.801 | 1.159 | 6.91 10⁻⁰¹ | 605 |
| VEGF | 1.418 | 1.210 | 1.661 | 1.52 10⁻⁰⁵ | 605 |
| STAT1 | 1.031 | 0.830 | 1.280 | 7.85 10⁻⁰¹ | 605 |
| CASP3 | 1.153 | 0.982 | 1.354 | 8.12 10⁻⁰² | 605 |

Table 7 corresponds to univariate analysis of different gene classifiers per molecular subgroup of untreated breast cancer patients. All signatures are considered here as continuous variables. GENE70=70 gene signature [10,4]; GENE76=76 gene signature [16,17]; P53=p53 signature [8]; WOUND=Wound response signature [12,18]; GGI=Genomic Grade Index [9]; ONCOTYPE=21-gene Recurrence Score [14]; IGS: 186-gene “invasiveness” gene signature [13].

In the multivariate analysis (n=775), proliferation [HR=2.48 (1.88-3.28), p=2 10⁻¹⁰], tumor invasion [1.41 (1.16-1.72), p=7 10⁻⁴], immune response [HR=0.72 (0.59-0.87), p=6 10⁻⁴], apoptosis [HR=1.18 (1.00-1.38), p=0.05], histological grade [HR=1.80 (1.12-2.88), p=0.02] were significantly associated with relapse free survival (RFS), with the proliferation module showing the largest HR and the most significant p-value among the molecular modules.

When the inventors considered the prototype genes alone, the performances were less pronounced compared to their respective modules, suggesting that averaging co-expressed genes into a module score is more stable and less dependent to cross-platform comparisons than the expression level of a singe gene.

### Molecular Module Scores, Clinico-Pathological Parameters and Prognosis (Prognostic) in the ESR1−/ERBB2−, ESR1+/ERBB2− and ERBB2+Molecular Subgroups

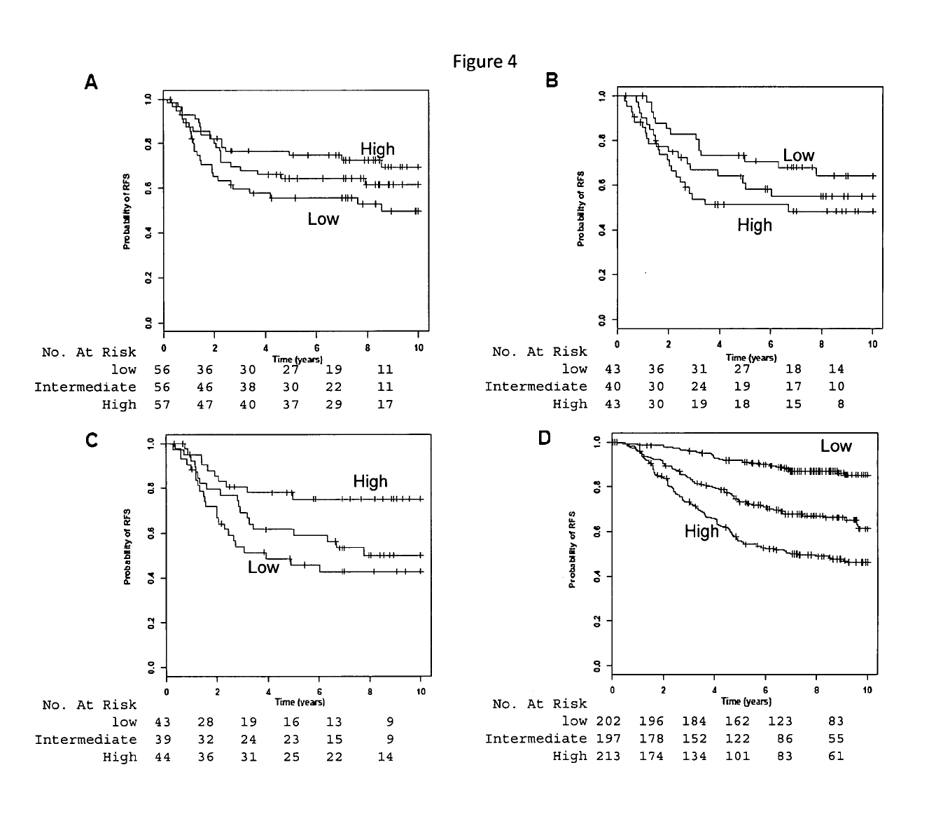

When investigating the prognostic value of the modules and clinico-pathological parameters according to the molecular subgroups defined above, the inventors observed that in the high risk ESR1−/ERBB2− subpopulation (n=169) only the immune response module showed a significant association with clinical outcome in both, univariate and multivariate analyses [HR=0.70 (0.50-0.98), p=0.04] (FIGS. 3-4).

Of interest, proliferation module lost its significance as almost all ER (ESR1) negative tumors showed high proliferation module scores.

In the ESR1+/ERBB2− subpopulation (n=531), age, tumor size and histological grade were associated with RFS, together with the HER2 (ERBB2), proliferation and angiogenesis modules. In multivariate analysis, only the proliferation module [HR=2.68 (2.02-3.55), p=9 10⁻¹²] and histological grade [HR=2.00 (1.18-3.37), p=0.01) remained significant, with the proliferation module having the highest HR and the most significant p-value.

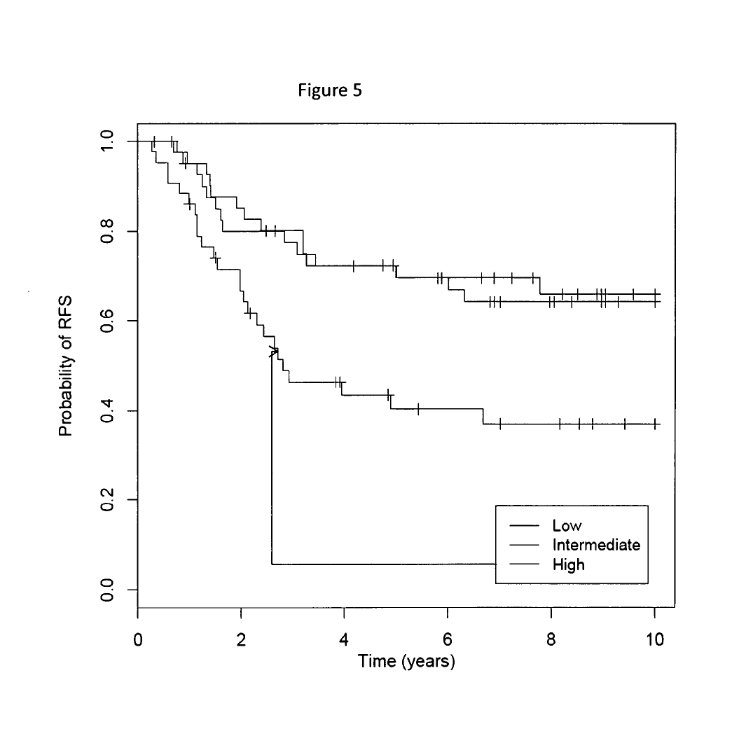

In the ERBB2+ tumors (n=126), nodal status, tumor invasion, angiogenesis and immune response modules or scores were significantly associated with RFS in the univariate model whereas only tumor invasion [HR=2.07 (1.32-3.25), p=0.001] and immune response [HR=0.56 (0.36-0.86), p=0.009] modules remained significantly associated with RFS in the multivariate model. The inventors then sought to combine these two variables in order to improve classification. Weights of +1 and −1 were used in the combination of the tumor invasion and immune response modules respectively. However, this simple combination did not significantly improve the classification of patients in the ERBB2+ subgroup with respect to prognosis (prognostic) as shown in FIG. 5.

### Dissecting Prognostic Gene Expression Signatures Using Molecular Modules

In order to investigate the biological meaning of the individual genes included in several published prognostic signatures (10, 4, 16, 17, 12, 18, 9, 14, 8, 13), the inventors applied the same comparison of linear models to several prognostic signatures in order to define which molecular category each individual gene included in these signatures belongs to. Table 8 illustrates the percentage of genes of each signature related to or specifically associated (value in brackets) with a particular prototype.

TABLE 8

| | | | AURKA | PLAU | VEGF | STAT1 | CASP3 |
|---|---|---|---|---|---|---|---|
| | ESR1 | ERBB2 | (Proliferation) | (Invasion) | (Angiogenesis) | (Immune response) | (Apoptosis) |
| GENE70 | 73% | 60% | 63% | 47% | 43% | 29% | 60% |
| | (10%) | (0%) | (14%) | (3%) | (0%) | (1%) | (0%) |
| GENE76 | 38% | 35% | 55% | 42% | 26% | 30% | 16% |
| | (3%) | (0%) | (16%) | (5%) | (1%) | (0%) | (1%) |
| P53 | 88% | 53% | 53% | 47% | 28% | 19% | 38% |
| | (34%) | (0%) | (16%) | (0%) | (0%) | (3%) | (0%) |
| WOUND | 42% | 30% | 52% | 39% | 35% | 30% | 40% |
| | (4%) | (0%) | (13%) | (3%) | (1%) | (0%) | (3%) |
| GGI | 73% | 37% | 99% | 64% | 43% | 43% | 30% |
| | (1%) | (2%) | (54%) | (0%) | (0%) | (0%) | (0%) |
| ONCOTYPE | 69% | 44% | 69% | 38% | 25% | 25% | 38% |
| | (19%) | (6%) | (13%) | (6%) | (0%) | (0%) | (0%) |
| IGS | 34% | 20% | 40% | 40% | 31% | 22% | 19% |
| | (10%) | (0%) | (10%) | (4%) | (1%) | (2%) | (0%) |

This analysis demonstrated that more than half of the genes in each signature investigated in this study were statistically associated with the proliferation prototype. Also the highest percentages of specific association, i.e. association with one prototype but not with the others, were also reported for AURKA, highlighting the importance of proliferation in several prognostic signatures.

The inventors further found that CD10 and/or PLAU signatures according to Tables 11 and/or 13 correlate with resistance to chemotherapy (anthracyclin).

The inventors use CD10 and/or PLAU signatures as diagnosis and/or to assist the choice of suitable medicine.

The inventors then went a step further by comparing the prognostic value of each molecular module of the “dissected” signature with the original one for three of the above reported prognostic gene signatures: the 70 gene [10,4], the 76 gene [16,17] and the genomic grade [9]. To do so, the inventors have used the TRANSBIG independent validation series of untreated primary breast cancer patients on which these signatures were computed using the original algorithms and micro-array platforms [5, 26], providing also the advantage that this population was not used for the development of any of these signatures. The inventors compared the hazard ratios for distant metastasis free survival for the group of genes from the original signatures, which were specifically associated with one of the prototypes, with the hazard ratio obtained with the original ones. Interestingly, as shown in FIG. 8, the performances of the proliferation modules were equivalent to the original signatures for all three investigated signatures, suggesting that proliferation might be the driving force. FIG. 8 represents forest plots showing the log 2 hazard ratios (and 95% CI) of the univariate analyses carried out on the TRANSBIG validation data [18-19] using the dissected signatures of GENE70=70 gene signature [1-2] (A), GENE76=76 gene signature [3-4] (B) and GGI=Genomic Grade Index [7] (C).

### Evaluating the Impact of the Prognostic Signatures in the Different Molecular Subgroups

In order to investigate which molecular subtype of breast cancer may benefit from these prognostic signatures the inventors analyzed the prognostic impact of the different gene signatures reported above in the different molecular subgroups defined by the ER (ESR1) and HER2 (ERBB2) molecular module scores. Since the exact algorithms for generating the different gene signatures cannot be applied on different micro-array platforms, the inventors decided to compute the classifiers as done for the module scores, using the direction of the association reported in the respective initial publications. Being concerned by the fact that a signed average might be less efficient than the original algorithm, the inventors conducted some comparison studies on original publications and found that the original and modified scores were highly correlated and that their performances were very similar. Since most predictors are often best described using unimodal distributions and since using dichotomized outcome variables may introduce a significant bias in comparing different prognostic signatures, the inventors considered here the different signatures as continuous variables. Also, it should be noted that given the application of robust scaling, the different signatures can be compared to one another.

The analysis of the prognostic power of these signatures by molecular subgroup, which was carried out only on patients which were not used in the development of these predictors, showed that the performance of these signatures seemed to be confined to the ESR1+/ERBB2-subgroup of patients (Table 9). Indeed the different signatures were not informative at all in the two other molecular subgroups.

TABLE 9

| | ESR1−/ERBB2− | | | ERBB2+ | | | ESR1+/ERBB2− | | |
|---|---|---|---|---|---|---|---|---|---|
| | HR | | Nr of | HR | | Nr of | HR | | Nr of |
| | (95% CI) | p-value | patients | (95% CI) | p-value | patients | (95% CI) | p-value | patients |
| GENE70 | 1.12 | 0.60 | 154 | 1.29 | 0.36 | 120 | 2.11 | 3 10⁻¹⁰ | 566 |
| | (0.73-1.72) | | | (0.75-2.20) | | | (1.67-2.66) | | |
| GENE76 | 1.30 | 0.32 | 99 | 0.81 | 0.42 | 85 | 1.52 | 2 10⁻⁵ | 422 |
| | (0.78-2.15) | | | (0.49-1.34) | | | (1.24-1.88) | | |
| P53 | 1.01 | 0.98 | 163 | 1.04 | 0.92 | 126 | 2.23 | 4 10⁻⁷ | 605 |
| | (0.42-2.42) | | | (0.51-2.11) | | | (1.64-3.03) | | |
| WOUND | 0.90 | 0.54 | 160 | 1.24 | 0.35 | 126 | 1.48 | 5 10⁻⁶ | 598 |
| | (0.65-1.26) | | | (0.79-1.93) | | | (1.25-1.75) | | |
| GGI | 0.78 | 0.38 | 165 | 0.79 | 0.48 | 126 | 3.16 | 2 10⁻¹⁹ | 598 |
| | (0.44-1.36) | | | (0.40-1.53) | | | (2.46-4.06) | | |
| ONCOTYPE | 0.86 | 0.74 | 156 | 1.00 | 1.00 | 126 | 4.79 | 3 10⁻²⁰ | 605 |
| | (0.36-2.08) | | | (0.50-2.02) | | | (3.43-6.68) | | |
| IGS | 1.08 | 0.70 | 169 | 0.96 | 0.85 | 126 | 2.12 | 6 10⁻¹³ | 605 |
| | (0.73-1.61) | | | (0.63-1.46) | | | (1.73-2.60) | | |

In this study, the inventors developed molecular modules representing several biological processes previously described in breast cancer, i.e. proliferation, tumor invasion, immune response, angiogenesis, apoptosis, as well as estrogen and HER2 (ERBB2) signalling. Although by dissecting breast cancer into its molecular components we simplified the nature of the disease, this study yielded a wealth of information regarding the understanding of the main biological processes involved in breast cancer and their impact on prognosis (prognostic).

The inventors first identified seven lists of genes representing the molecular modules. The module comprising the highest number of genes was the ER (ESR1) module (468 genes). This was not surprising since several publications on the molecular classification of breast cancer have repeatedly and consistently identified the estrogen receptor status of breast cancer as the main discriminator of expression subgroups [27, 28, 29, 30]. The second list with the highest number of genes was the one related to proliferation module (228 genes), which is consistent with the findings reported previously by Sotiriou et al. [30]. In contrast to these long lists, the modules reflecting angiogenesis, apoptosis and HER2 (ERBB2) signalling only ended up with a very limited number of genes, 13, 9 and 27 genes respectively. This can be partially explained by the fact that many genes associated with these modules were also associated with ER (ESR1) or proliferation (AURKA) and therefore not retained in the development of the other molecular modules.

The functional analysis of this molecular modules revealed also interesting information. As expected, many genes included in these modules were known to be associated with the chosen biological process. But many others, representing sometimes more than half of the module, were not yet reported to be related with breast cancer or were previously reported to be associated with another biological phenotype.

Investigating the relationship between traditional clinico-pathological markers and the different molecular modules revealed a positive association between the ER (ESR1) module and the age of the patient, an association which has been reported frequently for the protein levels of ER (ESR1) [31], as well as with the ER (ESR1) status, underlining a very good correlation between protein and expression levels of ER (ESR1).

Interestingly, the inventors observed a positive association between the HER2 (ERBB2) module and the ER (ESR1) protein expression status. As it has been suggested that the clinical efficacy of endocrine therapy might be compromised by the presence of HER2 (ERBB2) amplification or over-expression [32, 33, 34, 35, 36], the interrelationship of ER (ESR1) and HER2 (ERBB2) has come to have an important role in the management of breast cancer. Although the amplification/over-expression of HER2 (ERBB2) is generally inversely correlated with the expression of ER (ESR1), the precise extend of this correlation has only recently been reported by Lal et al. [37] in a large series of 3,655 breast cancer tumors using two of the standardized FDA-approved methods for HER2 (ERBB2) testing. Interestingly, they reported that almost half of the HER2 (ERBB2) positive tumors (49.1%) still expressed ER (ESR1). This supports the present finding that HER2 (ERBB2) module-positive tumors are associated with a positive ER (ESR1) protein status.

The inventors did not observe any association between the tumor invasion module (PLAU) and the clinico-pathological markers. This is in agreement with the study published by Leissner et al. [38], who investigated the mRNA expression of PLAU in lymph-node and hormone-receptor positive breast cancer.

Regarding the angiogenesis module, Bolat et al. also observed a positive correlation between VEGF and tumor size, although interestingly this finding seemed to be restricted to invasive ductal and not lobular carcinomas [39].

In a study involving 73 breast cancer patients, Widchwendter et al. found that high STAT1 activation was a significant predictor of good prognosis (prognostic) independent of the well-known prognosis (prognostic) markers and that the only parameter that correlated with STAT1 activation was the nodal status, the majority of tumors derived from LN-negative patients being associated with a high STAT1 activation [40], which is what the inventors also reported. This observation is in agreement with the fact that node-negative patients and high STAT1 are associated with a better prognosis (prognostic).

Breast cancer is a clinically heterogeneous disease. Several groups have consistently identified different molecular subclasses of breast cancer, with the basal-like (mostly ER (ESR1) and HER2 (ERBB2) negative) and HER2 (ERBB2) (mostly HER2 (ERBB2) amplified) subgroups showing the shortest relapse-free and overall survival, whereas the luminal-like type (estrogen receptor-positive) tumors had a more favorable clinical outcome (summarized in [41]). As we can no longer ignore the fact that these subgroups represent different types of breast cancer disease, we conducted the same analysis in the three subgroups identified by the main discriminators: ER (ESR1) and HER2 (ERBB2).

In the ESR1+/ERBB2− subgroup, proliferation module and histological grade were the two variables which remained associated with survival in the multivariate analysis, with the proliferation module having the most significant p-value. This is consistent with the finding that two clinically distinct ESR1-positive molecular subgroups can be defined by the genomic grade [6]. In the ERBB2+ subgroup, tumor invasion and immune response appeared to be the main processes associated with tumor progression. This finding supports that mRNA expression of PLAU was a powerful prognostic indicator in HER2 (ERBB2) positive tumors [42].

In the third subgroup (ESR1−/ERBB2−), only immune response appeared to predict prognosis (prognostic). It has been reported that tumors which do not express the hormone receptors and HER2 (ERBB2), commonly called the “triple-negative” or ‘basal-like” tumors, are more aggressive. Given their triple negative status, these patients cannot be treated with the conventional targeted therapies currently available for breast cancer, such as endocrine or HER2 (ERBB2)-targeted therapies, leaving chemotherapy as the only weapon.

In this context, several authors have suggested that chemotherapy might be more efficient in this subtype of the disease [43, 44]. However defining the optimal chemotherapy regimen remains controversial. Since BRCA1 pathway activity seems to be impaired in many of these tumors and since BRCA1 functions in DNA repair and cell cycle checkpoints, some authors have suggested that these tumors might be associated with sensitivity to DNA-damaging chemotherapy and may also be associated with resistance to spindle poisons [49]. In this study, the inventors showed that impaired immune response might be linked with the development of distant metastases (in this particular subgroup of patients). Indeed, high expression levels of the immune module (Tables 10 and 11) were associated with a significantly better outcome, both at the univariate and multivariate level.

It has been shown that STAT1 is particularly important in activating interferon-γ (IFN-γ) and its antitumor effects. In addition to inhibiting proliferation and survival, IFN-γ enhances the immunogenicity of tumor cells in part through enhancing STAT1-dependent expression of MHC proteins [46]. Based on this observation and the fact that an attenuated STAT1 signalling in tumors might be correlated with their malignant behavior, Lynch et al. recently postulated that enhancing gene transcription mediated by STAT1 may be an effective approach to cancer therapy [47]. Therefore, they screened 5,120 compounds and identified one molecule, 2-(1,8-naphthyridin-2-yl)phenol, that enhanced gene activation mediated by STAT1 over that seen with maximally efficacious concentration of IFN. Since STAT1 activation seems to be an important element in the killing of tumor cells in response to cytotoxic agents through repression of pro-survival genes and activation of apoptosis genes, its activation may be particularly important in patients receiving chemotherapy and particularly in these ESR1−/ERBB2− patients where most therapeutic approaches rely on cytotoxic agents that induce cell death in a nonspecific manner.

When the inventors dissected the main prognostic gene signatures reported so far in the literature to better understand their biological meaning, the inventors noticed that they were all composed by a significant proportion of proliferation-related genes. Also when the inventors compared the original signatures with their molecular modules in an independent series of patients, they noticed that the proliferation genes contained in the original signature were able to resume its prognostic performance. This underlines the fact that proliferation-related genes appear to be a common denominator of several existing prognostic gene expression signatures. Since defects in cell cycle deregulation are a fundamental characteristic of breast cancer, it is not surprising that these genes are involved in breast cancer prognosis (prognostic). Several studies showed indeed that increased expression of cell-cycle and proliferation-associated genes was correlated with poor outcome (reviewed in [48]). There are of course differences in the exact proliferation-associated genes, due to the difference in population analyzed or platform used. Although the use of proliferation-associated cell markers is not new, for example the protein expression levels of Ki67 and PCNA have already been used as prognostic markers for decades, gene expression profiling studies suggested that measuring proliferation using a more objective, automated and quantitative assay may be more robust compared to the less quantitative assays such as immunohistochemistry.

By investigating the prognostic ability of the main gene signatures reported so far according to the different breast cancer subtypes, the inventors have observed that the prognostic power of these signatures was limited to the ESR1+/ERBB2− molecular subgroup composed by estrogen receptor-positive patients. This is in agreement with the findings that: 1) proliferation seems to be the main contributor of these signatures and 2) the ESR1+/ERBB2− subgroup is the only molecular subgroup displaying a wide range of proliferation values.

This finding also emphasizes the need of additional prognostic markers for the other two molecular subgroups, and more specifically for the ESR1−/ERBB2− subgroup, which is associated with a poor prognosis (prognostic) and limited therapeutic options. Therefore, the inventors believe that by studying the immune response mechanisms in this particular subgroup of patients might help to better understand these tumors and to develop efficient targeted therapies.

To conclude, by identifying molecular modules representing the main biological mechanisms involved in breast cancer, the inventors were able to better characterize the biological foundation of the different prognostic signatures and to understand the mechanisms that trigger the different tumors to progress. These findings may help to define new clinico-genomic models and to identify new targets in the specific molecular subgroups, in order to make a step towards truly personalized medicine.

### Investigation of the Immune Response by Studying CD4+ Cells

The inventors have profiled CD4+ cells isolated from primary invasive ductal carcinomas. An unsupervised, hierarchical clustering algorithm allowed the inventors to distinguish two groups of tumors which were different regarding the pathways involved in immune response. Considering these immune pathways, 118 genes that are differentially expressed in tumor infiltrating CD4+ cells were identified and they generated a gene signature called “CD4 infiltrating tumor signature” (CD4ITS) that differs substantially from previously reported gene signatures in breast cancer. The relationship between CD4ITS and clinical outcome in more than 2600 patients listed in public datasets was also analysed. An important finding was that the CD4ITS was associated with the risk of metastasis in patients with subtype 1 breast carcinoma who are usually associated with the worst prognosis (prognostic).

### Materials and Methods

Patient's samples. Patients with invasive ductal breast carcinoma were recruited for the study. No patient had received any adjuvant systemic therapy. Human breast carcinoma tissues were obtained at the time of the surgery.

Patient datasets. Nine gene expression datasets obtained by micro-array analysis of tumor specimens from a total of 2641 patients with primary breast cancer were used: the dataset from van de Vijver 2002⁴, Buyse 2006⁵, Desmedt 2007²⁶, Loi 2007⁶, Sotiriou 2003⁷, Miller 2005⁸, Sotiriou 2006⁹, van't veer 2002¹⁰ and Sorlie 2003¹¹.

Isolation of CD4+ cells. A procedure to isolate CD4+ cells from ductal breast carcinoma was established. Briefly, carcinoma samples were mechanically dissociated using a scalpel. Fragments were incubated in 12-well culture dish with a mixture of Collagenase-Type 4 (Worthington) in x-vivo media (BioWhittaker) in a 37° C. incubator with 5% CO₂ with constant agitation for 20-60 min, depending of the size of the sample. Following dissociation, the digestion product were filtered through a nylon mesh using piston syringe and washed with x-vivo. The CD4+ cells were isolated form the unicellular suspension using Dynal® CD4 Positive Isolation Kit according to the manufacturer's instructions. The purity of the population was checked by flow cytometry.

Flow cytometry. To verify the quality of the T CD4+ cells isolation, the inventors have analyzed CD3, CD4 and CD8 surface expression by flow cytometry were analyzed. For this issue, beads of an aliquot of cells were detached according to the manufacturer's procedure. Briefly, 5 μl of each specific OItest conjugated antibody (Beckman Coulter) was added to the test tube containing cells resuspended in 50 μl HAFA buffer (RPMI 1640 without phenol red (BioWhittaker), 3% inactivated FBS, 20 mM NaN₃). The tube was vortexed and incubated for 30 minutes at 4° C., protected from the light. Cells were washed with PBS and fixed in 2% paraformaldehyde. Fluorescence analysis was performed by use of a FACSCalibur (BD Biosciences).

Isolation of RNA from lymphocytes. The RNA was extracted from fresh CD4+ cells using the phenol/chloroform procedure with TriPure Isolation Reagent (Roche Applied Science). Briefly, Tripure (1 ml) was added to each tube containing CD4+ cells. The tubes were vortexed and chloroform was added. Samples were placed on a Phase Lock Gel™ (Expenders) and centrifuged at 15682 rcf. The upper aqueous phase was removed and placed in a new tube. Isopropanol and glycogen were added, and then the tube was centrifuged to precipitate the RNA. The RNA pellet was washed twice with 75% ethanol, dried using Speedvack, and resuspended in nuclease-free water. The amount and the quality of RNA were respectively determined using the Nanodrop and the Agilent Capiler System.

Gene expression analysis. 10 patient's breast carcinomas with a sufficient amount of good quality RNA were isolated from purified CD4+ cells infiltrating primary tumour. Micro-array analysis was performed with Affymetrix U133Plus Genechips (Affymetrix). RNA two-cycle amplification, hybridation and scanning were done according to standard Affymetrix protocols. Image analysis and probe quantification was performed with the Affymetrix software that produced raw probe intensity data in the Affymetrix CEL files. The program RMA was use to normalise the data.

Statistical analysis. Considering the 10 expression profiles of CD4+ cells isolated from invasive ductal carcinomas, an unsupervised, hierarchical clustering was established. On the basis of the BioCarta pathways, the difference between the clusters was analysed. Genes involved in pathways related to the immune response and presenting a significant difference in the expression level were selected to compose the CD4ITS. A score, called the CD4ITS index (CD4ITSI) was introduced to summarize the similarity between the expression profile related to the immune reaction and the clinical outcome. Considering genes composing the CD4ITS, the CD4ITSI was defined as the sum of the fold change in upregulated genes subtracted from the sum of the fold change in downregulated genes. This score was then calculated for each patient listed in the datasets (n=2641). The datasets were exploited in whole or distinguishing the different subtypes of patient's tumors and/or the (un)administration of any therapy. Univariate and multivariate analyses of relapse with the use of the Cox proportional-hazards method were performed with the use of SPSS, version 15.0. To estimate the rates of overall metastasis-free survival along the time, the Kaplan-Meier method was used. In this issue, considered patient's data were then sorted by ascending score and a cutoff point was defined at 75^{th} percentile which divided the patients into two groups. Patients with low and high scores were assigned respectively to the group 1 and 2. Results were illustrated on survival curves.

### Results—Expression Profile of Tumor Infiltrating CD4+ Cells Differs According to the Er Status.

Using the micro-array technology, the genetic profiles of CD4+ cells isolated from 10 breast carcinomas was established namely 5 ER+ and 5 ER−. Regarding these profiles, an unsupervised clustering revealed 2 main clusters. Interestingly, these two clusters correspond practically to the ER status of the tumor. These clusters were very stable and reproducible using different clustering methods (centered, uncentered, completed or average linkage).

Localisation CD4+—Th1/Th2—Generation of the CD4+ infiltrating tumor signature (CD4ITS).

Considering the cellular pathways, the difference between the two main clusters which divide the expression profiles of the CD4+ cells infiltrating mammary tumors was examined. There were 37 statistically significant pathways which differed between the two clusters. Interestingly, 31 of those pathways were associated with immune reaction. A genetic signature, called the “CD4+ infiltrating tumor signature” (CD4ITS) was established. To access this issue, genes involved in these 31 immune pathways on the basis of a significant difference (p value<0.05) were selected.

The CD4ITS and outcome in breast cancer. The CD4ITS index (CD4ITSI) was calculated for each patient in the databases using the formula described in the patients and methods section. This index was tested for its association with clinical outcomes in a time relapse-free survival analysis using Cox proportional-hazards model in several datasets (n=2641). Considering this whole dataset, a low correlation was revealed between the CD4ITSI and the clinical outcome, with hazard ratios of 0.909 (95% CI, 0.840 to 0.984; P=0.018). Considering this result three subtypes of breast carcinomas, namely Esr1− Erbb2− (subtype 1 or “basal-like”), Erbb2+ (subtype2) and Esr1+ Erbb2− (subtype3 or “luminal”), were distinguish for discerning samples on the basis of these subtypes. Results showed a strong and statistically significant correlation between CD4ISI and the clinical outcome on subtype 1 breast carcinoma, with hazard ratios of 0.733 (95% CI, 0.620 to 0.867; P=0.000). A similar correlation was shown regarding the subtype 2 but with a slighter effect, with hazard ratios of 0.790 (95% CI, 0.635 to 0.982; P=0.033). No correlation was displayed with subtype 3, with hazard ratios of 0.920 (95% CI, 0.812 to 1,042; P=0.187).

To make further investigation among patient with subtype 1 breast carcinoma and to estimate the time relapse-free survival, the Kaplan-Meier method was used. In this issue, the patients were stratified according to the CD4ITS as described in the patients and methods section. The estimated 5-years rates of overall metastasis-free survival were 57.7% (CD4ITSI<75^{th} percentile) and 81.8% (CD4ITSI≧75^{th} D percentile).

The prognostic value of the CD4IS on treated and untreated patients with subtype 1 breast cancer was investigated. The prognostic value of CD4ITS is stronger on treated patients, with hazard ratios of 0.673 (95% CI, 0.512 to 0.884; P=0.004), than on untreated patients, with hazard ratios of 0.792 (95% CI, 0.638 to 0.983; P=0.034) (see table 4). The Kaplan-Meier method was performed as described above, the estimated 5-years rates of overall metastasis-free survival among treated and untreated patients were 48.7% (CD4ITSI<75^{th} percentile) and 81.5% (CD4ISI≧75^{th} percentile); 60.9% (CD4ITSI<75^{th} percentile) and 81.25% (CD4ISI≧75^{th} percentile) respectively.

The CD4ITS and other prognostic signatures. To estimate the robustness of the signature, according to the invention, the inventors have compared CD4ITS to the published predictive signatures, namely Wound¹², IGS¹³, Oncotype¹⁴, GGI⁹, Gene 70⁴, Gene 76¹⁵, on the treated and/or untreated patients with subtype 1 breast cancer. A Cox proportional-hazards model showed that CD4ITS was the unique signature which had a statistically significant predictive value among patient with subtype 1 breast cancer with hazard ratio of 0.733 (95% CI, 0.620 to 0.867; P=0.000). Discerning treated and untreated patients, the exclusive validity of the CD4ITS is strongly conserved among the treated one.

TABLE 2

| module | EntrezGene.ID | HUGO.gene.symbol | agilent | affy | coefficient | NMSE |
|---|---|---|---|---|---|---|
| ESR1 | 2099 | ESR1 | NM_000125 | 205225_at | 1 | 0 |
| | 23158 | TBC1D9 | AB020689 | 212956_at | 0.818853934 | 0.329519058 |
| | 2625 | GATA3 | NM_002051 | 209602_s_at | 0.808404454 | 0.340901046 |
| | 771 | CA12 | NM_001218 | 204508_s_at | 0.769664466 | 0.403723308 |
| | 3169 | FOXA1 | NM_004496 | 204667_at | 0.747740313 | 0.445912639 |
| | 4602 | MYB | NM_005375 | 204798_at | 0.724360247 | 0.476220193 |
| | 7802 | DNALI1 | NM_003462 | 205186_at | 0.722064641 | 0.476993136 |
| | 18 | ABAT | NM_020686 | 209459_s_at | 0.68431164 | 0.500878387 |
| | 7494 | XBP1 | NM_005080 | 200670_at | 0.706606341 | 0.504567097 |
| | 57758 | SCUBE2 | NM_020974 | 219197_s_at | 0.706307294 | 0.507028611 |
| | 2066 | ERBB4 | AF007153 | 214053_at | 0.705524131 | 0.50920309 |
| | 9 | NAT1 | NM_000662 | 214440_at | 0.68994857 | 0.524568765 |
| | 10551 | AGR2 | NM_006408 | 209173_at | 0.682493984 | 0.524896233 |
| | 987 | LRBA | M83822 | 212692_s_at | 0.667204458 | 0.545200585 |
| | 56521 | DNAJC12 | AF176012 | 218976_at | 0.654147619 | 0.552279601 |
| | 2203 | FBP1 | NM_000507 | 209696_at | 0.666017848 | 0.563765784 |
| | 51466 | EVL | NM_016337 | 217838_s_at | 0.653404963 | 0.564019798 |
| | 51442 | VGLL1 | NM_016267 | 215729_s_at | −0.66129561 | 0.567442475 |
| | 57496 | MKL2 | NM_014048 | 218259_at | 0.64903192 | 0.567499146 |
| | 7031 | TFF1 | NM_003225 | 205009_at | 0.6449711 | 0.567670532 |
| | 1153 | CIRBP | NM_001280 | 200810_s_at | 0.644376986 | 0.57712969 |
| | 26227 | PHGDH | NM_006623 | 201397_at | −0.64928809 | 0.582061385 |
| | 1555 | CYP2B6 | M29873 | 206754_s_at | 0.631227682 | 0.596212258 |
| | 6648 | SOD2 | NM_000636 | 215223_s_at | −0.62622708 | 0.605433039 |
| | 55638 | NA | NM_017786 | 218692_at | 0.629800859 | 0.605503031 |
| | 221061 | C10orf38 | AL050367 | 212771_at | −0.61911622 | 0.620120942 |
| | 7033 | TFF3 | NM_003226 | 204623_at | 0.616219874 | 0.620667764 |
| | 53335 | BCL11A | NM_018014 | 219497_s_at | −0.61751635 | 0.624593924 |
| | 79818 | ZNF552 | Contig43054 | 219741_x_at | 0.610820144 | 0.627481194 |
| | 57613 | KIAA1467 | AB040900 | 213234_at | 0.590842681 | 0.631251573 |
| | 8416 | ANXA9 | NM_003568 | 210085_s_at | 0.600083497 | 0.632229077 |
| | 582 | BBS1 | Contig1503_RC | 218471_s_at | 0.607975339 | 0.634990977 |
| | 54463 | NA | NM_019000 | 218532_s_at | 0.601669708 | 0.636624769 |
| | 55733 | HHAT | NM_018194 | 219687_at | 0.57829406 | 0.638592631 |
| | 2674 | GFRA1 | NM_005264 | 205696_s_at | 0.584823646 | 0.638780117 |
| | 4478 | MSN | NM_002444 | 200600_at | −0.59183487 | 0.643848416 |
| | 51097 | SCCPDH | NM_016002 | 201825_s_at | 0.594863448 | 0.646197689 |
| | 54502 | NA | NM_019027 | 218035_s_at | 0.597290216 | 0.649932337 |
| | 26018 | LRIG1 | AL117666 | 211596_s_at | 0.591723382 | 0.65103686 |
| | 55793 | FAM63A | NM_018379 | 221856_s_at | 0.586608892 | 0.655692588 |
| | 3868 | KRT16 | NM_005557 | 209800_at | −0.54949798 | 0.660555073 |
| | 54961 | SSH3 | NM_017857 | 219919_s_at | 0.580160177 | 0.662407239 |
| | 60481 | ELOVL5 | AF111849 | 208788_at | 0.582552358 | 0.663927448 |
| | 3667 | IRS1 | NM_005544 | 204686_at | 0.57148821 | 0.670004986 |
| | 83439 | TCF7L1 | Contig57725_RC | 221016_s_at | −0.57685166 | 0.670185709 |
| | 10950 | BTG3 | NM_006806 | 205548_s_at | −0.57803585 | 0.671668378 |
| | 3572 | IL6ST | NM_002184 | 204863_s_at | 0.566168955 | 0.672265327 |
| | 4783 | NFIL3 | NM_005384 | 203574_at | −0.55143972 | 0.674600099 |
| | 51161 | C3orf18 | NM_016210 | 219114_at | 0.553100882 | 0.675614902 |
| | 2296 | FOXC1 | NM_001453 | 213260_at | −0.56246613 | 0.677073594 |
| | 6664 | SOX11 | NM_003108 | 204914_s_at | −0.57838974 | 0.677177874 |
| | 5613 | PRKX | NM_005044 | 204061_at | −0.55539077 | 0.679650809 |
| | 8543 | LMO4 | NM_006769 | 209204_at | −0.56711672 | 0.680574997 |
| | 55686 | MREG | NM_018000 | 219648_at | 0.57186844 | 0.680694279 |
| | 8100 | IFT88 | NM_006531 | 204703_at | 0.55028445 | 0.682287138 |
| | 2617 | GARS | NM_002047 | 208693_s_at | −0.56419322 | 0.684354279 |
| | 3945 | LDHB | NM_002300 | 201030_x_at | −0.55557485 | 0.685360876 |
| | 8382 | NME5 | NM_003551 | 206197_at | 0.555210673 | 0.689486281 |
| | 10614 | HEXIM1 | NM_006460 | 202815_s_at | 0.5516074 | 0.690267345 |
| | 9633 | MTL5 | NM_004923 | 219786_at | 0.561763365 | 0.692112214 |
| | 2568 | GABRP | NM_014211 | 205044_at | −0.55883521 | 0.693312003 |
| | 23324 | MAN2B2 | AB023152 | 214703_s_at | 0.555058606 | 0.693977059 |
| | 55765 | C1orf106 | NM_018265 | 219010_at | −0.54180004 | 0.695474669 |
| | 5104 | SERPINA5 | J02639 | 209443_at | 0.552615794 | 0.696714554 |
| | 5174 | PDZK1 | NM_002614 | 205380_at | 0.546051055 | 0.697188944 |
| | 56674 | TMEM9B | Contig1462_RC | 218065_s_at | 0.528127412 | 0.698235582 |
| | 1054 | CEBPG | NM_001806 | 204203_at | −0.55314581 | 0.698369112 |
| | 9120 | SLC16A6 | NM_004694 | 207038_at | 0.548877174 | 0.701189497 |
| | 79641 | ROGDI | Contig292_RC | 218394_at | 0.54629249 | 0.701533185 |
| | 23303 | KIF13B | AF279865 | 202962_at | 0.541898896 | 0.702905771 |
| | 2173 | FABP7 | NM_001446 | 205029_s_at | −0.52941225 | 0.703037328 |
| | 23171 | GPD1L | D42047 | 212510_at | 0.544914666 | 0.705950088 |
| | 9674 | KIAA0040 | NM_014656 | 203143_s_at | 0.532088271 | 0.708978452 |
| | 27134 | TJP3 | NM_014428 | 213412_at | 0.542775525 | 0.710067869 |
| | 79921 | TCEAL4 | Contig3659_RC | 202371_at | 0.541970152 | 0.710331465 |
| | 54898 | ELOVL2 | AL080199 | 213712_at | 0.52925655 | 0.710508034 |
| | 1345 | COX6C | NM_004374 | 201754_at | 0.539941313 | 0.710572245 |
| | 5937 | RBMS1 | NM_016839 | 207266_x_at | −0.53974436 | 0.711344043 |
| | 400451 | NA | AL110139 | 51158_at | 0.537420183 | 0.716062616 |
| | 3898 | LAD1 | NM_005558 | 203287_at | −0.53550815 | 0.716693669 |
| | 2530 | FUT8 | NM_004480 | 203988_s_at | 0.505530007 | 0.718532442 |
| | 51306 | C5orf5 | NM_016603 | 218518_at | 0.528812601 | 0.719378071 |
| | 25837 | RAB26 | NM_014353 | 219562_at | 0.526164961 | 0.719523191 |
| | 10982 | MAPRE2 | X94232 | 202501_at | −0.51938230 | 0.721044346 |
| | 1632 | DCI | NM_001919 | 209759_s_at | 0.5213171 | 0.721375708 |
| | 7905 | REEP5 | M73547 | 208873_s_at | 0.525130991 | 0.725825747 |
| | 1101 | CHAD | NM_001267 | 206869_at | 0.526770704 | 0.726408365 |
| | 323 | APBB2 | U62325 | 213419_at | 0.507242904 | 0.729583221 |
| | 28958 | CCDC56 | NM_014019 | 218026_at | 0.523641457 | 0.729997843 |
| | 1476 | CSTB | NM_000100 | 201201_at | −0.52228528 | 0.730310348 |
| | 9435 | CHST2 | NM_004267 | 203921_at | −0.52396710 | 0.730941092 |
| | 7371 | UCK2 | NM_012474 | 209825_s_at | −0.51709149 | 0.733658287 |
| | 2737 | GLI3 | NM_000168 | 205201_at | 0.521494671 | 0.733707267 |
| | 8685 | MARCO | NM_006770 | 205819_at | −0.51838499 | 0.73371596 |
| | 3295 | HSD17B4 | NM_000414 | 201413_at | 0.49793269 | 0.738043938 |
| | 11013 | TMSL8 | D82345 | 205347_s_at | −0.48243814 | 0.738461069 |
| | 51604 | PIGT | NM_015937 | 217770_at | 0.514231244 | 0.738548025 |
| | 6663 | SOX10 | NM_006941 | 209842_at | −0.52250076 | 0.739074324 |
| | 85377 | MICALL1 | Contig55538_RC | 221779_at | −0.51653462 | 0.739527411 |
| | 58495 | OVOL2 | AL079276 | 211778_s_at | 0.509854248 | 0.740100478 |
| | 1116 | CHI3L1 | NM_001276 | 209395_at | −0.50752539 | 0.741531574 |
| | 11001 | SLC27A2 | NM_003645 | 205768_s_at | 0.504487267 | 0.743254132 |
| | 25841 | ABTB2 | AL050374 | 213497_at | −0.50152319 | 0.744291557 |
| | 64080 | RBKS | Contig54394_RC | 57540_at | 0.501098938 | 0.744631881 |
| | 375035 | SFT2D2 | AL035297 | 214838_at | −0.48888167 | 0.745192165 |
| | 10479 | SLC9A6 | NM_006359 | 203909_at | −0.46218527 | 0.746780768 |
| | 5002 | SLC22A18 | NM_002555 | 204981_at | 0.498450997 | 0.747634385 |
| | 8645 | KCNK5 | NM_003740 | 219615_s_at | −0.50676541 | 0.748157343 |
| | 79885 | HDAC11 | AL137362 | 219847_at | 0.503640516 | 0.748262024 |
| | 11254 | SLC6A14 | NM_007231 | 219795_at | −0.46793656 | 0.748739207 |
| | 122616 | C14orf79 | AF038188 | 213512_at | 0.508580125 | 0.749420609 |
| | 79650 | C16orf57 | Contig56298_RC | 218060_s_at | −0.51270039 | 0.749551419 |
| | 23321 | TRIM2 | AB011089 | 202341_s_at | −0.50510712 | 0.749962222 |
| | 23327 | NEDD4L | AB007899 | 212448_at | 0.502371307 | 0.750281297 |
| | 22977 | AKR7A3 | NM_012067 | 206469_x_at | 0.49969396 | 0.750370918 |
| | 8581 | LY6D | X82693 | 206276_at | −0.49652701 | 0.750473705 |
| | 8842 | PROM1 | NM_006017 | 204304_s_at | −0.49873779 | 0.750894641 |
| | 4953 | ODC1 | NM_002539 | 200790_at | −0.50017862 | 0.752229895 |
| | 55544 | RBM38 | X75315 | 212430_at | −0.48523095 | 0.752354883 |
| | 55663 | ZNF446 | NM_017908 | 219900_s_at | 0.502643541 | 0.752376668 |
| | 27124 | PIB5PA | U45975 | 213651_at | 0.493911581 | 0.753414597 |
| | 6715 | SRD5A1 | NM_001047 | 211056_s_at | −0.49787464 | 0.756655029 |
| | 51809 | GALNT7 | NM_017423 | 218313_s_at | 0.491503578 | 0.757011056 |
| | 89927 | C16orf45 | Contig1239_RC | 212736_at | 0.491495819 | 0.757310477 |
| | 1827 | DSCR1 | NM_004414 | 208370_s_at | −0.45318343 | 0.757687519 |
| | 51706 | CYB5R1 | NM_016243 | 202263_at | 0.480014471 | 0.75876488 |
| | 3383 | ICAM1 | NM_000201 | 202638_s_at | −0.4921546 | 0.759111299 |
| | 5806 | PTX3 | NM_002852 | 206157_at | −0.50095406 | 0.759263083 |
| | 9501 | RPH3AL | NM_006987 | 221614_s_at | 0.489345723 | 0.759692293 |
| | 3613 | IMPA2 | NM_014214 | 203126_at | −0.49271114 | 0.759753232 |
| | 7568 | ZNF20 | AL080125 | 213916_at | 0.474191523 | 0.760393024 |
| | 6280 | S100A9 | NM_002965 | 203535_at | −0.48574767 | 0.761593701 |
| | 22929 | SEPHS1 | NM_012247 | 208941_s_at | −0.49031224 | 0.762710604 |
| | 81563 | C1orf21 | Contig56307 | 221272_s_at | 0.48956231 | 0.762763451 |
| | 1389 | CREBL2 | NM_001310 | 201990_s_at | 0.468866383 | 0.764274897 |
| | 1410 | CRYAB | NM_001885 | 209283_at | −0.49071498 | 0.764626005 |
| | 10884 | MRPS30 | NM_016640 | 218398_at | 0.479596064 | 0.765432562 |
| | 55614 | C20orf23 | AK000142 | 219570_at | 0.486726442 | 0.765836231 |
| | 1824 | DSC2 | Contig49790_RC | 204750_s_at | −0.48878224 | 0.765994757 |
| | 7851 | MALL | U17077 | 209373_at | −0.48905517 | 0.766316309 |
| | 2743 | GLRB | NM_000824 | 205280_at | 0.480525648 | 0.766572036 |
| | 427 | ASAH1 | NM_004315 | 210980_s_at | 0.474147175 | 0.766857518 |
| | 5241 | PGR | NM_000926 | 208305_at | 0.507968301 | 0.767931467 |
| | 51364 | ZMYND10 | NM_015896 | 205714_s_at | 0.465885335 | 0.768320131 |
| | 6926 | TBX3 | NM_016569 | 219682_s_at | 0.467758204 | 0.768972653 |
| | 5193 | PEX12 | NM_000286 | 205094_at | 0.465534987 | 0.771299562 |
| | 8531 | CSDA | NM_003651 | 201161_s_at | −0.48379436 | 0.771700739 |
| | 23 | ABCF1 | AF027302 | 200045_at | −0.45941767 | 0.771727802 |
| | 7545 | ZIC1 | NM_003412 | 206373_at | −0.47973354 | 0.77245107 |
| | 819 | CAMLG | NM_001745 | 203538_at | 0.470697705 | 0.772933304 |
| | 2947 | GSTM3 | NM_000849 | 202554_s_at | 0.477492539 | 0.773863567 |
| | 5825 | ABCD3 | NM_002858 | 202850_at | 0.478558366 | 0.774199051 |
| | 5860 | QDPR | NM_000320 | 209123_at | 0.466880459 | 0.77694304 |
| | 59342 | SCPEP1 | Contig51742_RC | 218217_at | −0.46539062 | 0.777429767 |
| | 51806 | CALML5 | NM_017422 | 220414_at | −0.43692661 | 0.777841349 |
| | 79603 | LASS4 | Contig55127_RC | 218922_s_at | 0.44467496 | 0.780061636 |
| | 21 | ABCA3 | NM_001089 | 204343_at | 0.476768516 | 0.780354714 |
| | 54847 | SIDT1 | NM_017699 | 219734_at | 0.457175309 | 0.78051878 |
| | 8537 | BCAS1 | NM_003657 | 204378_at | 0.471260926 | 0.781068878 |
| | 10874 | NMU | NM_006681 | 206023_at | −0.40879552 | 0.782327854 |
| | 54149 | C21orf91 | NM_017447 | 220941_s_at | −0.45741133 | 0.782940362 |
| | 9929 | JOSD1 | NM_014876 | 201751_at | −0.45878624 | 0.785508213 |
| | 5317 | PKP1 | NM_000299 | 221854_at | −0.47574048 | 0.785750041 |
| | 7388 | UQCRH | NM_006004 | 202233_s_at | −0.46334012 | 0.786324045 |
| | 64764 | CREB3L2 | AL080209 | 212345_s_at | −0.44888154 | 0.78771472 |
| | 10127 | ZNF263 | NM_005741 | 203707_at | 0.459983171 | 0.78860236 |
| | 80347 | COASY | U18919 | 201913_s_at | 0.441985485 | 0.788930057 |
| | 126353 | C19orf21 | Contig53480_RC | 212925_at | 0.448608295 | 0.789172076 |
| | 50865 | HEBP1 | NM_015987 | 218450_at | 0.446561227 | 0.790515478 |
| | 54812 | AFTPH | Contig44143 | 217939_s_at | 0.455170453 | 0.791035737 |
| | 64087 | MCCC2 | AL079298 | 209624_s_at | 0.462857334 | 0.792137211 |
| | 8884 | SLC5A6 | AL096737 | 204087_s_at | −0.43982908 | 0.793363126 |
| | 5269 | SERPINB6 | S69272 | 211474_s_at | 0.46113414 | 0.793737295 |
| | 4321 | MMP12 | NM_002426 | 204580_at | −0.44026565 | 0.793907251 |
| | 8190 | MIA | NM_006533 | 206560_s_at | −0.42956164 | 0.794003971 |
| | 6769 | STAC | NM_003149 | 205743_at | −0.46154415 | 0.794035744 |
| | 51368 | TEX264 | NM_015926 | 218548_x_at | 0.435409448 | 0.794574725 |
| | 23541 | SEC14L2 | NM_012429 | 204541_at | 0.449863872 | 0.795691113 |
| | 9185 | REPS2 | NM_004726 | 205645_at | 0.442965761 | 0.796203486 |
| | 185 | AGTR1 | NM_000685 | 205357_s_at | 0.448719626 | 0.796491882 |
| | 7368 | UGT8 | NM_003360 | 208358_s_at | −0.47320635 | 0.797181557 |
| | 399665 | FAM102A | AL049365 | 212400_at | 0.426089803 | 0.797887209 |
| | 12 | SERPINA3 | NM_001085 | 202376_at | 0.430128647 | 0.798346485 |
| | 55975 | KLHL7 | NM_018846 | 220238_s_at | −0.44715312 | 0.799331759 |
| | 25864 | ABHD14A | AL050015 | 210006_at | 0.431227602 | 0.799391044 |
| | 4851 | NOTCH1 | NM_017617 | 218902_at | −0.44628024 | 0.800453543 |
| | 9091 | PIGQ | NM_004204 | 204144_s_at | 0.448022351 | 0.800799077 |
| | 1299 | COL9A3 | NM_001853 | 204724_s_at | −0.43453156 | 0.801359118 |
| | 2800 | GOLGA1 | NM_002077 | 203384_s_at | 0.432417726 | 0.801979288 |
| | 8326 | FZD9 | NM_003508 | 207639_at | −0.46571299 | 0.802324839 |
| | 6376 | CX3CL1 | NM_002996 | 203687_at | −0.44647627 | 0.802408813 |
| | 8399 | PLA2G10 | NM_003561 | 207222_at | 0.441846629 | 0.802595278 |
| | 5327 | PLAT | NM_000931 | 201860_s_at | 0.446276147 | 0.802779242 |
| | 22885 | ABLIM3 | NM_014945 | 205730_s_at | 0.446223817 | 0.803580219 |
| | 11094 | C9orf7 | NM_017586 | 219223_at | 0.438954737 | 0.803900187 |
| | 5321 | PLA2G4A | M68874 | 210145_at | −0.42416523 | 0.80390189 |
| | 57348 | TTYH1 | NM_020659 | 219415_at | −0.45165274 | 0.805615356 |
| | 6787 | NEK4 | NM_003157 | 204634_at | 0.438354592 | 0.807293759 |
| | 123872 | LRRC50 | AL137334 | 222068_s_at | 0.423132817 | 0.808146112 |
| | 10421 | CD2BP2 | NM_006110 | 202257_s_at | 0.438472091 | 0.809185652 |
| | 5971 | RELB | NM_006509 | 205205_at | −0.42058475 | 0.810752119 |
| | 6833 | ABCC8 | NM_000352 | 210246_s_at | 0.43299799 | 0.811094072 |
| | 11122 | PTPRT | NM_007050 | 205948_at | 0.441958947 | 0.811634327 |
| | 23650 | TRIM29 | NM_012101 | 211002_s_at | −0.41153904 | 0.812560427 |
| | 79629 | OCEL1 | Contig49281_RC | 205441_at | 0.402331924 | 0.812866251 |
| | 8722 | CTSF | NM_003793 | 203657_s_at | 0.436109995 | 0.813444547 |
| | 57110 | HRASLS | NM_020386 | 219984_s_at | −0.43040468 | 0.813917579 |
| | 6697 | SPR | NM_003124 | 203458_at | 0.374042555 | 0.815469964 |
| | 2919 | CXCL1 | NM_001511 | 204470_at | −0.43103914 | 0.815720462 |
| | 27250 | PDCD4 | AL049932 | 212593_s_at | 0.42229844 | 0.815720916 |
| | 23245 | ASTN2 | AB014534 | 215407_s_at | 0.432272945 | 0.81655549 |
| | 10265 | IRX5 | NM_005853 | 210239_at | 0.444238765 | 0.816746883 |
| | 2824 | GPM6B | Contig448_RC | 209170_s_at | −0.42759793 | 0.8168277 |
| | 10644 | IGF2BP2 | NM_006548 | 218847_at | −0.40137448 | 0.817753304 |
| | 7436 | VLDLR | NM_003383 | 209822_s_at | −0.41016150 | 0.81824919 |
| | 25825 | BACE2 | NM_012105 | 217867_x_at | −0.42961248 | 0.818674706 |
| | 10827 | C5orf3 | NM_018691 | 218588_s_at | 0.427773891 | 0.819304526 |
| | 4828 | NMB | M21551 | 205204_at | −0.42674501 | 0.820247788 |
| | 6720 | SREBF1 | NM_004176 | 202308_at | 0.417450053 | 0.820708855 |
| | 10477 | UBE2E3 | NM_006357 | 210024_s_at | −0.42413489 | 0.822164226 |
| | 3066 | HDAC2 | NM_001527 | 201833_at | −0.42527142 | 0.822454328 |
| | 55224 | ETNK2 | NM_018208 | 219268_at | 0.400594749 | 0.823435185 |
| | 875 | CBS | NM_000071 | 212816_s_at | −0.36357167 | 0.823556622 |
| | 3872 | KRT17 | NM_000422 | 205157_s_at | −0.39795768 | 0.82378018 |
| | 753 | C18orf1 | NM_004338 | 207996_s_at | 0.423862631 | 0.823845166 |
| | 136 | ADORA2B | NM_000676 | 205891_at | −0.42306361 | 0.823856862 |
| | 2013 | EMP2 | NM_001424 | 204975_at | 0.421077857 | 0.824624291 |
| | 1917 | EEF1A2 | NM_001958 | 204540_at | 0.430874995 | 0.825239707 |
| | 3576 | IL8 | NM_000584 | 202859_x_at | −0.42263800 | 0.825795247 |
| | 419 | ART3 | NM_001179 | 210147_at | −0.43304415 | 0.825917814 |
| | 55650 | PIGV | NM_017837 | 51146_at | 0.420582519 | 0.826931805 |
| | 23107 | MRPS27 | D87453 | 212145_at | 0.406366641 | 0.826940683 |
| | 25818 | KLK5 | NM_012427 | 222242_s_at | −0.41340419 | 0.827115168 |
| | 8309 | ACOX2 | NM_003500 | 205364_at | 0.408316599 | 0.827876009 |
| | 1047 | CLGN | NM_004362 | 205830_at | 0.369392157 | 0.82901223 |
| | 10002 | NR2E3 | NM_014249 | 208388_at | 0.407775212 | 0.830043531 |
| | 60487 | TRMT11 | Contig54010_RC | 218877_s_at | −0.40566142 | 0.830431941 |
| | 10656 | KHDRBS3 | NM_006558 | 209781_s_at | −0.40340408 | 0.831344622 |
| | 55240 | STEAP3 | NM_018234 | 218424_s_at | −0.41466295 | 0.83324228 |
| | 3315 | HSPB1 | NM_001540 | 201841_s_at | 0.406168651 | 0.834031319 |
| | 10273 | STUB1 | NM_005861 | 217934_x_at | 0.413376875 | 0.834700244 |
| | 2171 | FABP5 | NM_001444 | 202345_s_at | −0.41219044 | 0.835111923 |
| | 55184 | C20orf12 | NM_018152 | 219951_s_at | 0.39674387 | 0.835120573 |
| | 5783 | PTPN13 | NM_006264 | 204201_s_at | 0.392109759 | 0.835383296 |
| | 1877 | E4F1 | NM_004424 | 218524_at | 0.400337951 | 0.83577919 |
| | 11098 | PRSS23 | NM_007173 | 202458_at | 0.408630816 | 0.836021917 |
| | 10202 | DHRS2 | NM_005794 | 214079_at | 0.394698247 | 0.836221587 |
| | 80223 | RAB11FIP1 | Contig1682_RC | 219681_s_at | 0.409041709 | 0.836355265 |
| | 79627 | OGFRL1 | Contig39960_RC | 219582_at | −0.41147589 | 0.836715105 |
| | 6948 | TCN2 | NM_000355 | 204043_at | −0.40164819 | 0.836747162 |
| | 3097 | HIVEP2 | NM_006734 | 212641_at | −0.40364447 | 0.838742793 |
| | 8985 | PLOD3 | NM_001084 | 202185_at | −0.40629339 | 0.83937633 |
| | 3892 | KRT86 | X99142 | 215189_at | −0.40898783 | 0.839394877 |
| | 10575 | CCT4 | NM_006430 | 200877_at | −0.40322219 | 0.839667184 |
| | 51004 | COQ6 | NM_015940 | 218760_at | 0.40443291 | 0.839743802 |
| | 4071 | TM4SF1 | M90657 | 215034_s_at | −0.4024996 | 0.839926234 |
| | 1718 | DHCR24 | D13643 | 200862_at | 0.380176977 | 0.839949625 |
| | 1381 | CRABP1 | NM_004378 | 205350_at | −0.40429027 | 0.8409904 |
| | 9368 | SLC9A3R1 | NM_004252 | 201349_at | 0.405852497 | 0.841380916 |
| | 92104 | TTC30A | AL049329 | 213679_at | 0.403451511 | 0.841551015 |
| | 9518 | GDF15 | NM_004864 | 221577_x_at | 0.402707288 | 0.841948716 |
| | 6364 | CCL20 | NM_004591 | 205476_at | −0.36319472 | 0.842019711 |
| | 3306 | HSPA2 | U56725 | 211538_s_at | 0.395674599 | 0.842245746 |
| | 79605 | PGBD5 | Contig53598_RC | 219225_at | −0.40705584 | 0.84277541 |
| | 23336 | DMN | AB002351 | 212730_at | −0.39034362 | 0.843586584 |
| | 1356 | CP | NM_000096 | 204846_at | −0.40404337 | 0.843884436 |
| | 54619 | CCNJ | NM_019084 | 219470_x_at | −0.38111750 | 0.844401655 |
| | 9200 | PTPLA | NM_014241 | 219654_at | −0.39972249 | 0.844778941 |
| | 51302 | CYP39A1 | NM_016593 | 220432_s_at | −0.33695618 | 0.844975117 |
| | 5191 | PEX7 | NM_000288 | 205420_at | 0.396991099 | 0.845179405 |
| | 706 | TSPO | NM_007311 | 202096_s_at | −0.39169845 | 0.845341528 |
| | 7159 | TP53BP2 | NM_005426 | 203120_at | −0.39572610 | 0.845767077 |
| | 55218 | EXDL2 | NM_018199 | 218363_at | 0.401498328 | 0.846250153 |
| | 79669 | C3orf52 | Contig53814_RC | 219474_at | 0.388442276 | 0.846776039 |
| | 10140 | TOB1 | NM_005749 | 202704_at | 0.367622466 | 0.84725245 |
| | 11226 | GALNT6 | Contig49342_RC | 219956_at | 0.395283101 | 0.847253692 |
| | 6652 | SORD | NM_003104 | 201563_at | 0.394652204 | 0.847767541 |
| | 3418 | IDH2 | NM_002168 | 210046_s_at | −0.40013914 | 0.847804159 |
| | 10200 | MPHOSPH6 | NM_005792 | 203740_at | −0.39554753 | 0.848141674 |
| | 7345 | UCHL1 | NM_004181 | 201387_s_at | −0.37679195 | 0.84953539 |
| | 6564 | SLC15A1 | NM_005073 | 207254_at | −0.34318347 | 0.850903361 |
| | 54458 | PRR13 | NM_018457 | 217794_at | 0.392279425 | 0.850920162 |
| | 51103 | NDUFAF1 | NM_016013 | 204125_at | 0.353122452 | 0.85105789 |
| | 11042 | NA | NM_006780 | 215043_s_at | 0.388381527 | 0.851937806 |
| | 10040 | TOM1L1 | NM_005486 | 204485_s_at | 0.382624539 | 0.852751814 |
| | 1117 | CHI3L2 | U49835 | 213060_s_at | −0.37689236 | 0.853033349 |
| | 112398 | EGLN2 | NM_017555 | 220956_s_at | 0.392095205 | 0.853446237 |
| | 9258 | MFHAS1 | NM_004225 | 213457_at | −0.32447140 | 0.85362056 |
| | 374 | AREG | NM_001657 | 205239_at | 0.375610148 | 0.854146851 |
| | 2982 | GUCY1A3 | NM_000856 | 221942_s_at | −0.38254572 | 0.854163644 |
| | 688 | KLF5 | NM_001730 | 209211_at | −0.39113342 | 0.854558871 |
| | 1960 | EGR3 | NM_004430 | 206115_at | 0.373008187 | 0.85611316 |
| | 7993 | UBXD6 | NM_005671 | 215983_s_at | 0.382878926 | 0.856242287 |
| | 25823 | TPSG1 | NM_012467 | 220339_s_at | 0.373878408 | 0.856591509 |
| | 4485 | MST1 | L11924 | 205614_x_at | 0.357450422 | 0.857946991 |
| | 23528 | ZNF281 | NM_012482 | 218401_s_at | 0.379127283 | 0.858339794 |
| | 1672 | DEFB1 | NM_005218 | 210397_at | −0.39076646 | 0.858685673 |
| | 28960 | DCPS | NM_014026 | 218774_at | −0.38267717 | 0.858774643 |
| | 5268 | SERPINB5 | NM_002639 | 204855_at | −0.35802733 | 0.859249445 |
| | 934 | CD24 | NM_013230 | 209772_s_at | −0.36282951 | 0.86062728 |
| | 55450 | CAMK2N1 | NM_018584 | 218309_at | 0.370660238 | 0.860945792 |
| | 6261 | RYR1 | NM_000540 | 205485_at | −0.35082856 | 0.861340834 |
| | 2627 | GATA6 | NM_005257 | 210002_at | −0.37081347 | 0.862200066 |
| | 57180 | ACTR3B | NM_020445 | 218868_at | −0.38659759 | 0.862506996 |
| | 4036 | LRP2 | NM_004525 | 205710_at | 0.350254766 | 0.86266905 |
| | 29116 | MYLIP | NM_013262 | 220319_s_at | 0.373793594 | 0.862681243 |
| | 57211 | GPR126 | AL080079 | 213094_at | −0.37693751 | 0.862687147 |
| | 4435 | CITED1 | NM_004143 | 207144_s_at | 0.375304645 | 0.862985246 |
| | 54913 | RPP25 | NM_017793 | 219143_s_at | −0.37237191 | 0.86390199 |
| | 9982 | FGFBP1 | NM_005130 | 205014_at | −0.33016268 | 0.864260466 |
| | 11170 | FAM107A | NM_007177 | 209074_s_at | −0.35901803 | 0.864884193 |
| | 3294 | HSD17B2 | NM_002153 | 204818_at | −0.38270805 | 0.866150203 |
| | 6583 | SLC22A4 | NM_003059 | 205896_at | 0.323184257 | 0.866415185 |
| | 79170 | ATAD4 | Contig61975 | 219127_at | 0.373271428 | 0.867669413 |
| | 79745 | CLIP4 | Contig48631 | 219944_at | −0.27836229 | 0.86848439 |
| | 2813 | GP2 | NM_016295 | 214324_at | 0.346238895 | 0.868853586 |
| | 6723 | SRM | NM_003132 | 201516_at | −0.34578620 | 0.870266606 |
| | 1360 | CPB1 | NM_001871 | 205509_at | 0.346493776 | 0.871724386 |
| | 5016 | OVGP1 | NM_002557 | 205432_at | 0.340204667 | 0.872087776 |
| | 5271 | SERPINB8 | NM_002640 | 206034_at | −0.35808395 | 0.872952965 |
| | 347902 | AMIGO2 | Contig49079_RC | 222108_at | 0.36104055 | 0.87334578 |
| | 79719 | NA | Contig57044_RC | 202851_at | 0.364020628 | 0.874136088 |
| | 55258 | NA | NM_018271 | 219044_at | 0.358273868 | 0.874179008 |
| | 8563 | THOC5 | NM_003678 | 209418_s_at | −0.35724536 | 0.874354782 |
| | 83464 | APH1B | Contig53314_RC | 221036_s_at | 0.38272656 | 0.874569471 |
| | 23532 | PRAME | NM_006115 | 204086_at | −0.35189188 | 0.87568013 |
| | 6834 | SURF1 | NM_003172 | 204295_at | 0.360498545 | 0.876816575 |
| | 6019 | RLN2 | NM_005059 | 214519_s_at | 0.340131262 | 0.877580596 |
| | 214 | ALCAM | NM_001627 | 201951_at | 0.357195699 | 0.878486882 |
| | 55333 | SYNJ2BP | NM_018373 | 219156_at | 0.354152982 | 0.878595717 |
| | 10525 | HYOU1 | NM_006389 | 200825_s_at | −0.35389917 | 0.879309158 |
| | 2232 | FDXR | NM_004110 | 207813_s_at | 0.357851956 | 0.88094545 |
| | 274 | BIN1 | NM_004305 | 210202_s_at | −0.36200933 | 0.8810547 |
| | 10307 | APBB3 | NM_006051 | 204650_s_at | 0.346101202 | 0.882638244 |
| | 8986 | RPS6KA4 | NM_003942 | 204632_at | −0.33810477 | 0.882825424 |
| | 56938 | ARNTL2 | NM_020183 | 220658_s_at | −0.35442683 | 0.883130457 |
| | 9510 | ADAMTS1 | NM_006988 | 222162_s_at | −0.31714081 | 0.883576407 |
| | 2770 | GNAI1 | NM_002069 | 209576_at | −0.34021112 | 0.883662467 |
| | 4350 | MPG | NM_002434 | 203686_at | 0.341676941 | 0.884004809 |
| | 863 | CBFA2T3 | NM_005187 | 208056_s_at | 0.344392794 | 0.884416124 |
| | 2891 | GRIA2 | NM_000826 | 205358_at | 0.325402619 | 0.884813944 |
| | 10309 | UNG2 | X52486 | 210021_s_at | 0.340406908 | 0.884921127 |
| | 7037 | TFRC | NM_003234 | 207332_s_at | −0.33653368 | 0.884923454 |
| | 3574 | IL7 | NM_000880 | 206693_at | −0.34389077 | 0.885221043 |
| | 55293 | UEVLD | NM_018314 | 220775_s_at | 0.344688842 | 0.885938381 |
| | 27165 | GLS2 | NM_013267 | 205531_s_at | 0.254837341 | 0.886441129 |
| | 55188 | RIC8B | NM_018157 | 219446_at | 0.342486332 | 0.887434273 |
| | 11202 | KLK8 | NM_007196 | 206125_s_at | −0.35998705 | 0.887541757 |
| | 51181 | DCXR | NM_016286 | 217973_at | 0.299804251 | 0.88771423 |
| | 827 | CAPN6 | NM_014289 | 202965_s_at | −0.32896134 | 0.888075448 |
| | 390 | RND3 | Contig3682_RC | 212724_at | −0.33533047 | 0.888607585 |
| | 54438 | GFOD1 | NM_018988 | 219821_s_at | −0.33775830 | 0.889053494 |
| | 10079 | ATP9A | AB014511 | 212062_at | 0.328282857 | 0.889255142 |
| | 4285 | MIPEP | NM_005932 | 36830_at | 0.356463366 | 0.889469146 |
| | 8324 | FZD7 | NM_003507 | 203706_s_at | −0.33206439 | 0.889884855 |
| | 9052 | GPRC5A | NM_003979 | 203108_at | 0.346433922 | 0.890040223 |
| | 9508 | ADAMTS3 | AB002364 | 214913_at | −0.29195187 | 0.890309433 |
| | 10519 | CIB1 | NM_006384 | 201953_at | 0.318187791 | 0.890742687 |
| | 7138 | TNNT1 | NM_003283 | 213201_s_at | 0.331611482 | 0.891033522 |
| | 51735 | RAPGEF6 | NM_016340 | 219112_at | 0.326267887 | 0.89116631 |
| | 54970 | TTC12 | NM_017868 | 219587_at | 0.291552597 | 0.891346796 |
| | 2591 | GALNT3 | NM_004482 | 203397_s_at | −0.34242172 | 0.891358691 |
| | 2348 | FOLR1 | NM_000802 | 204437_s_at | −0.32727835 | 0.891730283 |
| | 2954 | GSTZ1 | NM_001513 | 209531_at | 0.334740431 | 0.891823109 |
| | 23318 | ZCCHC11 | D83776 | 212704_at | −0.28744690 | 0.891980859 |
| | 10267 | RAMP1 | NM_005855 | 204916_at | 0.331220193 | 0.892185659 |
| | 25984 | KRT23 | NM_015515 | 218963_s_at | −0.33772871 | 0.89242928 |
| | 6496 | SIX3 | NM_005413 | 206634_at | −0.26458260 | 0.892787299 |
| | 786 | CACNG1 | NM_000727 | 206612_at | 0.325288477 | 0.893132764 |
| | 22976 | PAXIP1 | U80735 | 212825_at | 0.314975901 | 0.893439408 |
| | 283232 | TMEM80 | Contig52603_RC | 221951_at | 0.334733545 | 0.894635943 |
| | 629 | CFB | NM_001710 | 202357_s_at | 0.325947876 | 0.895246912 |
| | 7286 | TUFT1 | NM_020127 | 205807_s_at | 0.324287679 | 0.8957374 |
| | 5562 | PRKAA1 | NM_006251 | 209799_at | −0.27248266 | 0.897249406 |
| | 9851 | KIAA0753 | NM_014804 | 204711_at | 0.33776741 | 0.897696217 |
| | 79622 | C16orf33 | Contig52526_RC | 218493_at | 0.313083514 | 0.898920401 |
| | 55316 | RSAD1 | NM_018346 | 218307_at | 0.329901495 | 0.898981065 |
| | 6271 | S100A1 | NM_006271 | 205334_at | −0.32519543 | 0.899120454 |
| | 55859 | BEX1 | NM_018476 | 218332_at | 0.315589822 | 0.899579486 |
| | 3595 | IL12RB2 | NM_001559 | 206999_at | −0.34467894 | 0.900222341 |
| | 5100 | PCDH8 | NM_002590 | 206935_at | −0.35519567 | 0.900356755 |
| | 2861 | GPR37 | NM_005302 | 209631_s_at | −0.31562942 | 0.902920283 |
| | 26278 | SACS | NM_014363 | 213262_at | −0.29589301 | 0.903024533 |
| | 55506 | H2AFY2 | NM_018649 | 218445_at | −0.31488076 | 0.904286521 |
| | 64215 | DNAJC1 | Contig3538_RC | 218409_s_at | 0.309391077 | 0.904704283 |
| | 3096 | HIVEP1 | NM_002114 | 204512_at | −0.30420168 | 0.905214361 |
| | 23059 | CLUAP1 | AB014543 | 204577_s_at | 0.308081913 | 0.905659063 |
| | 79602 | ADIPOR2 | Contig41209_RC | 201346_at | 0.294636455 | 0.905943382 |
| | 56683 | C21orf59 | NM_017835 | 218123_at | 0.30298336 | 0.906330205 |
| | 22943 | DKK1 | NM_012242 | 204602_at | −0.31707767 | 0.906552011 |
| | 6277 | S100A6 | NM_014624 | 217728_at | −0.31127446 | 0.906567008 |
| | 65983 | GRAMD3 | AL157454 | 218706_s_at | −0.31070593 | 0.906845373 |
| | 4255 | MGMT | NM_002412 | 204880_at | 0.306014355 | 0.906934039 |
| | 10406 | WFDC2 | NM_006103 | 203892_at | 0.310318913 | 0.908053059 |
| | 3760 | KCNJ3 | NM_002239 | 207142_at | 0.289824264 | 0.90907496 |
| | 23552 | CCRK | NM_012119 | 205271_s_at | 0.281880641 | 0.910569983 |
| | 9722 | NOS1AP | AB007933 | 215153_at | 0.229340894 | 0.911497251 |
| | 23613 | PRKCBP1 | AB032951 | 209049_s_at | 0.299807266 | 0.911563244 |
| | 202 | AIM1 | U83115 | 212543_at | −0.28250629 | 0.912039471 |
| | 51207 | DUSP13 | NM_016364 | 219963_at | 0.295957672 | 0.913470799 |
| | 83988 | NCALD | AF052142 | 211685_s_at | −0.27863454 | 0.913549975 |
| | 2920 | CXCL2 | NM_002089 | 209774_x_at | −0.23251798 | 0.913929307 |
| | 8870 | IER3 | NM_003897 | 201631_s_at | 0.293240479 | 0.914353765 |
| | 55245 | C20orf44 | NM_018244 | 217935_s_at | 0.292257279 | 0.914633438 |
| | 6666 | SOX12 | NM_006943 | 204432_at | 0.288976299 | 0.91494091 |
| | 80279 | CDK5RAP3 | AK000260 | 218740_s_at | 0.295086243 | 0.915477346 |
| | 1644 | DDC | NM_000790 | 205311_at | −0.25539982 | 0.915582189 |
| | 5441 | POLR2L | NM_021128 | 202586_at | 0.290705454 | 0.915792241 |
| | 9022 | CLIC3 | NM_004669 | 219529_at | −0.29342331 | 0.915932573 |
| | 7769 | ZNF226 | NM_015919 | 219603_s_at | 0.291518083 | 0.91618188 |
| | 27239 | GPR162 | NM_019858 | 205056_s_at | 0.267327121 | 0.916259358 |
| | 26504 | CNNM4 | NM_020184 | 218900_at | 0.299283579 | 0.916676204 |
| | 3400 | ID4 | NM_001546 | 209291_at | −0.29901729 | 0.917135234 |
| | 1733 | DIO1 | NM_000792 | 206457_s_at | 0.277146054 | 0.918178806 |
| | 25915 | C3orf60 | AL049955 | 209177_at | 0.275728009 | 0.918466799 |
| | 1525 | CXADR | NM_001338 | 203917_at | −0.29399348 | 0.918866262 |
| | 1475 | CSTA | NM_005213 | 204971_at | −0.29629654 | 0.919065795 |
| | 2155 | F7 | NM_019616 | 207300_s_at | 0.291791149 | 0.919083227 |
| | 4188 | MDFI | NM_005586 | 205375_at | −0.29462263 | 0.919236535 |
| | 3622 | ING2 | NM_001564 | 205981_s_at | 0.290622475 | 0.919303599 |
| | 25980 | C20orf4 | NM_015511 | 218089_at | 0.203116625 | 0.919391746 |
| | 8310 | ACOX3 | NM_003501 | 204242_s_at | 0.287582101 | 0.919961112 |
| | 54820 | NDE1 | NM_017668 | 218414_s_at | 0.282080137 | 0.920079592 |
| | 5816 | PVALB | NM_002854 | 205336_at | 0.227358785 | 0.920203757 |
| | 60686 | C14orf93 | Contig51318_RC | 219009_at | 0.24607044 | 0.920539974 |
| | 8792 | TNFRSF11A | NM_003839 | 207037_at | −0.30152349 | 0.920541992 |
| | 54894 | RNF43 | NM_017763 | 218704_at | 0.280441269 | 0.923270824 |
| | 5737 | PTGFR | NM_000959 | 207177_at | −0.2231448 | 0.924206492 |
| | 1501 | CTNND2 | U96136 | 209618_at | 0.273276047 | 0.924383316 |
| | 7764 | ZNF217 | NM_006526 | 203739_at | 0.276000692 | 0.925380013 |
| | 8405 | SPOP | NM_003563 | 208927_at | 0.270754072 | 0.926506674 |
| | 1847 | DUSP5 | NM_004419 | 209457_at | 0.277032448 | 0.927166495 |
| | 4488 | MSX2 | NM_002449 | 205555_s_at | 0.295463635 | 0.927546165 |
| | 7163 | TPD52 | NM_005079 | 201691_s_at | 0.263461652 | 0.927805212 |
| | 25790 | CCDC19 | NM_012337 | 220308_at | 0.286351098 | 0.928605166 |
| | 5803 | PTPRZ1 | NM_002851 | 204469_at | −0.26445918 | 0.92970977 |
| | 23635 | SSBP2 | NM_012446 | 203787_at | 0.261272248 | 0.930412837 |
| | 6548 | SLC9A1 | S68616 | 209453_at | 0.266541892 | 0.930417948 |
| | 8187 | ZNF239 | NM_005674 | 206261_at | 0.273064581 | 0.931123654 |
| | 2588 | GALNS | NM_000512 | 206335_at | −0.23243233 | 0.93213956 |
| | 54903 | MKS1 | NM_017777 | 218630_at | 0.248040673 | 0.932362145 |
| | 55163 | PNPO | Contig55446_RC | 218511_s_at | 0.255506984 | 0.932823779 |
| | 55101 | NA | NM_018035 | 218038_at | 0.266549718 | 0.933387577 |
| | 4682 | NUBP1 | NM_002484 | 203978_at | 0.244519893 | 0.934015928 |
| | 3779 | KCNMB1 | NM_004137 | 209948_at | −0.21564509 | 0.934522794 |
| | 64849 | SLC13A3 | AF154121 | 205243_at | −0.27379455 | 0.935284703 |
| | 4691 | NCL | NM_005381 | 200610_s_at | −0.25948109 | 0.93550478 |
| | 64428 | NARFL | Contig41536_RC | 218742_at | 0.203857245 | 0.935624333 |
| | 23266 | LPHN2 | NM_012302 | 206953_s_at | −0.25295037 | 0.936162229 |
| | 29104 | N6AMT1 | NM_013240 | 220311_at | 0.222484457 | 0.937942569 |
| | 1783 | DYNC1LI2 | NM_006141 | 203590_at | −0.24622451 | 0.938320864 |
| | 8987 | NA | NM_003943 | 203986_at | 0.243504322 | 0.938630895 |
| | 79852 | ABHD9 | Contig21225_RC | 220013_at | −0.27078394 | 0.93887984 |
| | 57586 | SYT13 | AB037848 | 221859_at | 0.239472393 | 0.939365745 |
| | 8785 | MATN4 | NM_003833 | 207123_s_at | −0.20822884 | 0.939574568 |
| | 10331 | B3GNT3 | NM_014256 | 204856_at | −3 | 0.940573085 |
| | 5357 | PLS1 | NM_002670 | 205190_at | 0.247326218 | 0.940664991 |
| | 54880 | BCOR | Contig26100_RC | 219433_at | 0.229605443 | 0.942981745 |
| | 55790 | NA | NM_018371 | 219049_at | −0.25042614 | 0.943118658 |
| | 4139 | MARK1 | NM_018650 | 221047_s_at | −0.24475937 | 0.944329845 |
| | 81539 | SLC38A1 | Contig58438_RC | 218237_s_at | 0.241702504 | 0.945111586 |
| | 10810 | WASF3 | NM_006646 | 204042_at | −0.18215567 | 0.945444166 |
| | 926 | CD8B | NM_004931 | 215332_s_at | −0.24348476 | 0.945464604 |
| | 50805 | IRX4 | NM_016358 | 220225_at | −0.23224835 | 0.945544554 |
| | 58513 | EPS15L1 | NM_021235 | 221056_x_at | 0.233246267 | 0.94611709 |
| | 6304 | SATB1 | NM_002971 | 203408_s_at | −0.23571514 | 0.946625307 |
| | 79446 | WDR25 | Contig50337_RC | 219609_at | 0.208642099 | 0.948915101 |
| | 23366 | NA | AB020702 | 213424_at | 0.234295176 | 0.948952138 |
| | 55699 | IARS2 | NM_018060 | 217900_at | 0.230870685 | 0.949477716 |
| ERBB2 | 2064 | ERBB2 | NM_004448 | 216836_s_at | 1 | 0 |
| | 93210 | PERLD1 | Contig56503_RC | 221811_at | 0.907758645 | 0.17200875 |
| | 5709 | PSMD3 | NM_002809 | 201388_at | 0.679856111 | 0.551760856 |
| | 5409 | PNMT | NM_002686 | 206793_at | 0.65236504 | 0.581082444 |
| | 55876 | GSDML | NM_018530 | 219233_s_at | 0.551201489 | 0.701042445 |
| | 22794 | CASC3 | NM_007359 | 207842_s_at | 0.475868476 | 0.791261269 |
| | 3927 | LASP1 | NM_006148 | 200618_at | 0.465455223 | 0.802630026 |
| | 147179 | WIPF2 | U90911 | 212051_at | 0.438708817 | 0.803363538 |
| | 55040 | EPN3 | NM_017957 | 220318_at | 0.402128957 | 0.840891081 |
| | 5245 | PHB | NM_002634 | 200659_s_at | 0.397536834 | 0.852777893 |
| | 9635 | CLCA2 | NM_006536 | 217528_at | 0.36055161 | 0.867650117 |
| | 3227 | HOXC11 | NM_014212 | 206745_at | 0.312754199 | 0.881082423 |
| | 29095 | ORMDL2 | NM_014182 | 218556_at | 0.349298325 | 0.883214676 |
| | 5909 | RAP1GAP | NM_002885 | 203911_at | 0.337350258 | 0.889359836 |
| | 1573 | CYP2J2 | NM_000775 | 205073_at | 0.309379585 | 0.903278515 |
| | 26154 | ABCA12 | AL080207 | 215465_at | 0.292060066 | 0.908124968 |
| | 3081 | HGD | NM_000187 | 205221_at | 0.302330606 | 0.90880385 |
| | 8804 | CREG1 | NM_003851 | 201200_at | −0.29666354 | 0.915982859 |
| | 9914 | ATP2C2 | NM_014861 | 206043_s_at | 0.291958436 | 0.917143657 |
| | 5129 | PCTK3 | AL161977 | 214797_s_at | −0.29470259 | 0.919581811 |
| | 54793 | KCTD9 | NM_017634 | 218823_s_at | −0.28572478 | 0.919693777 |
| | 404093 | CUEDC1 | NM_017949 | 219468_s_at | 0.320633179 | 0.925765463 |
| | 3675 | ITGA3 | NM_002204 | 201474_s_at | 0.274007124 | 0.927570492 |
| | 55129 | TMEM16K | NM_018075 | 218910_at | 0.256032493 | 0.92892133 |
| | 24147 | FJX1 | NM_014344 | 219522_at | −0.25223514 | 0.939735137 |
| | 1048 | CEACAM5 | M29540 | 201884_at | 0.25663632 | 0.947093755 |
| | 9572 | NR1D1 | X72631 | 204760_s_at | 0.244126274 | 0.94968023 |
| | 51375 | SNX7 | NM_015976 | 205573_s_at | −0.23406410 | 0.949762889 |
| AURKA | 6790 | AURKA | NM_003600 | 208079_s_at | 1 | 0 |
| | 11065 | UBE2C | NM_007019 | 202954_at | 0.820863855 | 0.332578721 |
| | 9133 | CCNB2 | NM_004701 | 202705_at | 0.79214599 | 0.375663771 |
| | 1058 | CENPA | NM_001809 | 204962_s_at | 0.786068713 | 0.378411034 |
| | 332 | BIRC5 | NM_001168 | 202095_s_at | 0.785737371 | 0.385905904 |
| | 11004 | KIF2C | NM_006845 | 209408_at | 0.776738323 | 0.403529163 |
| | 10112 | KIF20A | NM_005733 | 218755_at | 0.7580889 | 0.420402209 |
| | 991 | CDC20 | NM_001255 | 202870_s_at | 0.743241214 | 0.435115841 |
| | 2305 | FOXM1 | U74612 | 202580_x_at | 0.743383899 | 0.439906192 |
| | 891 | CCNB1 | Contig56843_RC | 214710_s_at | 0.749756817 | 0.441921351 |
| | 22974 | TPX2 | AB024704 | 210052_s_at | 0.748568487 | 0.468134359 |
| | 9088 | PKMYT1 | NM_004203 | 204267_x_at | 0.702883844 | 0.47437898 |
| | 54478 | FAM64A | NM_019013 | 221591_s_at | 0.685128928 | 0.487318586 |
| | 4751 | NEK2 | NM_002497 | 204641_at | 0.718457153 | 0.487941235 |
| | 24137 | KIF4A | NM_012310 | 218355_at | 0.710510621 | 0.488813369 |
| | 23397 | NCAPH | D38553 | 212949_at | 0.72007551 | 0.490967285 |
| | 9319 | TRIP13 | U96131 | 204033_at | 0.710205816 | 0.499972805 |
| | 4085 | MAD2L1 | NM_002358 | 203362_s_at | 0.695603942 | 0.517656017 |
| | 9156 | EXO1 | NM_006027 | 204603_at | 0.673978083 | 0.540280713 |
| | 10615 | SPAG5 | NM_006461 | 203145_at | 0.670442201 | 0.550833392 |
| | 7083 | TK1 | NM_003258 | 202338_at | 0.643196792 | 0.554895627 |
| | 6491 | STIL | NM_003035 | 205339_at | 0.679351067 | 0.561436112 |
| | 6241 | RRM2 | NM_001034 | 209773_s_at | 0.663496582 | 0.564978476 |
| | 55839 | CENPN | NM_018455 | 219555_s_at | 0.665830165 | 0.566600085 |
| | 7298 | TYMS | NM_001071 | 202589_at | 0.65945932 | 0.568519762 |
| | 641 | BLM | NM_000057 | 205733_at | 0.649401343 | 0.584673125 |
| | 4171 | MCM2 | NM_004526 | 202107_s_at | 0.635855115 | 0.597104864 |
| | 1164 | CKS2 | NM_001827 | 204170_s_at | 0.614902417 | 0.610429408 |
| | 79682 | MLF1IP | Contig64688 | 218883_s_at | 0.624317967 | 0.615339427 |
| | 10129 | FRY | U50534 | 204072_s_at | −0.59404899 | 0.652505205 |
| | 51659 | GINS2 | NM_016095 | 221521_s_at | 0.582355702 | 0.652817049 |
| | 10212 | DDX39 | NM_005804 | 201584_s_at | 0.568291258 | 0.657312844 |
| | 3925 | STMN1 | NM_005563 | 200783_s_at | 0.589613162 | 0.657518464 |
| | 79801 | SHCBP1 | Contig34952 | 219493_at | 0.585901802 | 0.661475953 |
| | 3014 | H2AFX | NM_002105 | 205436_s_at | 0.579987829 | 0.666254194 |
| | 10535 | RNASEH2A | NM_006397 | 203022_at | 0.580753923 | 0.666515392 |
| | 5984 | RFC4 | NM_002916 | 204023_at | 0.575746351 | 0.671194217 |
| | 55970 | GNG12 | AL049367 | 212294_at | −0.56373935 | 0.68491997 |
| | 1033 | CDKN3 | NM_005192 | 209714_s_at | 0.575815638 | 0.6918622 |
| | 55388 | MCM10 | NM_018518 | 220651_s_at | 0.572262092 | 0.69399602 |
| | 55257 | C20orf20 | NM_018270 | 218586_at | 0.553371639 | 0.695442511 |
| | 1163 | CKS1B | NM_001826 | 201897_s_at | 0.545468556 | 0.698030816 |
| | 8914 | TIMELESS | NM_003920 | 203046_s_at | 0.559966788 | 0.704852194 |
| | 54821 | NA | NM_017669 | 219650_at | 0.506228567 | 0.70697648 |
| | 23371 | TENC1 | AB028998 | 212494_at | −0.54033843 | 0.719688949 |
| | 8544 | PIR | NM_003662 | 207469_s_at | 0.51732303 | 0.722573201 |
| | 8317 | CDC7 | AF015592 | 204510_at | 0.522596999 | 0.730034447 |
| | 2331 | FMOD | NM_002023 | 202709_at | −0.49793008 | 0.730688731 |
| | 51512 | GTSE1 | NM_016426 | 215942_s_at | 0.522293944 | 0.737008012 |
| | 6424 | SFRP4 | NM_003014 | 204051_s_at | −0.50398156 | 0.739316208 |
| | 55353 | LAPTM4B | NM_018407 | 208029_s_at | 0.510974612 | 0.741225782 |
| | 8404 | SPARCL1 | NM_004684 | 200795_at | −0.50844548 | 0.744694596 |
| | 990 | CDC6 | NM_001254 | 203967_at | 0.503962062 | 0.748292813 |
| | 7043 | TGFB3 | NM_003239 | 209747_at | −0.50101461 | 0.750780117 |
| | 11047 | ADRM1 | NM_007002 | 201281_at | 0.481127919 | 0.752181185 |
| | 58190 | CTDSP1 | NM_021198 | 217844_at | −0.48706893 | 0.757675543 |
| | 79838 | TMC5 | Contig45537_RC | 219580_s_at | −0.48922140 | 0.762742558 |
| | 84823 | LMNB2 | M94362 | 216952_s_at | 0.492907473 | 0.765450281 |
| | 83989 | C5orf21 | AF070617 | 212936_at | −0.48676706 | 0.766896872 |
| | 1793 | DOCK1 | NM_001380 | 203187_at | −0.48337292 | 0.768557986 |
| | 9358 | ITGBL1 | NM_004791 | 205422_s_at | −0.43649111 | 0.769646328 |
| | 8836 | GGH | NM_003878 | 203560_at | 0.484685676 | 0.769709668 |
| | 57088 | PLSCR4 | NM_020353 | 218901_at | −0.482651 | 0.770237787 |
| | 6642 | SNX1 | AL050148 | 213364_s_at | −0.46500284 | 0.770486626 |
| | 4969 | OGN | NM_014057 | 218730_s_at | −0.46695975 | 0.770624576 |
| | 90627 | STARD13 | AL049801 | 213103_at | −0.48080449 | 0.770936403 |
| | 11260 | XPOT | NM_007235 | 212160_at | 0.472165093 | 0.772199633 |
| | 22827 | NA | AF114818 | 209899_s_at | 0.477068606 | 0.773496315 |
| | 9793 | CKAP5 | D43948 | 212832_s_at | 0.466604145 | 0.783735263 |
| | 2791 | GNG11 | NM_004126 | 204115_at | −0.43671582 | 0.785914493 |
| | 55247 | NEIL3 | NM_018248 | 219502_at | 0.387791125 | 0.785965193 |
| | 10234 | LRRC17 | NM_005824 | 205381_at | −0.47039399 | 0.78807293 |
| | 9353 | SLIT2 | NM_004787 | 209897_s_at | −0.44561465 | 0.7891295 |
| | 1841 | DTYMK | NM_012145 | 203270_at | 0.453199348 | 0.790596547 |
| | 9631 | NUP155 | NM_004298 | 206550_s_at | 0.463044246 | 0.793503739 |
| | 5424 | POLD1 | NM_002691 | 203422_at | 0.436580111 | 0.79418075 |
| | 6631 | SNRPC | NM_003093 | 201342_at | 0.439785378 | 0.794257849 |
| | 10186 | LHFP | NM_005780 | 218656_s_at | −0.45165415 | 0.800444579 |
| | 4521 | NUDT1 | NM_002452 | 204766_s_at | 0.452653404 | 0.801745536 |
| | 3479 | IGF1 | X57025 | 209540_at | −0.44609695 | 0.802085779 |
| | 4172 | MCM3 | NM_002388 | 201555_at | 0.449081552 | 0.802988628 |
| | 2205 | FCER1A | NM_002001 | 211734_s_at | −0.44806141 | 0.803412984 |
| | 55732 | C1orf112 | NM_018186 | 220840_s_at | 0.42605845 | 0.806117986 |
| | 9077 | DIRAS3 | NM_004675 | 215506_s_at | −0.44520841 | 0.806296741 |
| | 5557 | PRIM1 | NM_000946 | 205053_at | 0.449712622 | 0.807788703 |
| | 54963 | UCKL1 | NM_017859 | 218533_s_at | 0.435505247 | 0.808482789 |
| | 54512 | EXOSC4 | NM_019037 | 218695_at | 0.438481818 | 0.808756437 |
| | 79901 | CYBRD1 | Contig52737_RC | 217889_s_at | −0.44056444 | 0.809596032 |
| | 10161 | P2RY5 | NM_005767 | 218589_at | −0.44050726 | 0.811708835 |
| | 29097 | CNIH4 | NM_014184 | 218728_s_at | 0.405953438 | 0.816190894 |
| | 6513 | SLC2A1 | NM_006516 | 201250_s_at | 0.43835292 | 0.81712218 |
| | 51123 | ZNF706 | NM_016096 | 218059_at | 0.428982832 | 0.819079758 |
| | 857 | CAV1 | NM_001753 | 203065_s_at | −0.42094884 | 0.825361732 |
| | 51110 | LACTB2 | NM_016027 | 218701_at | 0.384063357 | 0.829135483 |
| | 51204 | CCDC44 | NM_016360 | 221069_s_at | 0.414669919 | 0.829701293 |
| | 54845 | RBM35A | NM_017697 | 219121_s_at | 0.404725151 | 0.831774816 |
| | 283 | ANG | NM_001145 | 205141_at | −0.41211819 | 0.834366082 |
| | 79652 | C16orf30 | Contig26371_RC | 219315_s_at | −0.40614066 | 0.835774978 |
| | 56944 | OLFML3 | NM_020190 | 218162_at | −0.39638017 | 0.835872435 |
| | 3297 | HSF1 | NM_005526 | 202344_at | 0.393113682 | 0.836172966 |
| | 27235 | COQ2 | NM_015697 | 213379_at | 0.394874544 | 0.838129037 |
| | 2487 | FRZB | NM_001463 | 203698_s_at | −0.40214515 | 0.842301657 |
| | 3251 | HPRT1 | NM_000194 | 202854_at | 0.401889944 | 0.842800545 |
| | 5119 | PCOLN3 | NM_002768 | 201933_at | 0.401736559 | 0.842814242 |
| | 6839 | SUV39H1 | NM_003173 | 218619_s_at | 0.396921778 | 0.845003472 |
| | 27303 | RBMS3 | NM_014483 | 206767_at | −0.38281855 | 0.845114787 |
| | 10468 | FST | NM_013409 | 204948_s_at | −0.37734935 | 0.851436401 |
| | 26289 | AK5 | NM_012093 | 219308_s_at | −0.39522360 | 0.852323896 |
| | 55038 | CDCA4 | NM_017955 | 218399_s_at | 0.386970228 | 0.853046269 |
| | 7283 | TUBG1 | NM_001070 | 201714_at | 0.377543673 | 0.856260137 |
| | 23212 | RRS1 | D25218 | 209567_at | 0.381084547 | 0.859588011 |
| | 65094 | JMJD4 | Contig52872_RC | 218560_s_at | 0.386721791 | 0.860408119 |
| | 55379 | LRRC59 | NM_018509 | 222231_s_at | 0.366371991 | 0.860584113 |
| | 10956 | NA | NM_006812 | 215399_s_at | −0.29552516 | 0.860849464 |
| | 51022 | GLRX2 | NM_016066 | 219933_at | 0.373617007 | 0.862306014 |
| | 54915 | YTHDF1 | NM_017798 | 221741_s_at | 0.367355134 | 0.86250978 |
| | 54861 | SNRK | D43636 | 209481_at | −0.36814557 | 0.864874681 |
| | 79000 | C1orf135 | Contig25124_RC | 220011_at | 0.34885364 | 0.865018496 |
| | 79776 | ZFHX4 | Contig48790_RC | 219779_at | −0.37598813 | 0.866552699 |
| | 79971 | GPR177 | Contig53944_RC | 221958_s_at | −0.34276730 | 0.866720045 |
| | 7718 | ZNF165 | NM_003447 | 206683_at | 0.338079971 | 0.869974566 |
| | 201254 | STRA13 | U95006 | 209478_at | 0.363815143 | 0.871696996 |
| | 1848 | DUSP6 | NM_001946 | 208893_s_at | −0.34350182 | 0.871975414 |
| | 9037 | SEMA5A | NM_003966 | 205405_at | −0.37577719 | 0.872467328 |
| | 5433 | POLR2D | NM_004805 | 203664_s_at | 0.390567073 | 0.873347886 |
| | 29087 | THYN1 | NM_014174 | 218491_s_at | −0.32498531 | 0.874699946 |
| | 79864 | C11orf63 | Contig27559_RC | 220141_at | −0.35818107 | 0.875013566 |
| | 358 | AQP1 | NM_000385 | 209047_at | −0.32225578 | 0.876068416 |
| | 6634 | SNRPD3 | NM_004175 | 202567_at | 0.356764571 | 0.876553009 |
| | 2621 | GAS6 | NM_000820 | 202177_at | −0.35061025 | 0.876900397 |
| | 56270 | WDR45L | NM_019613 | 209076_s_at | 0.337179642 | 0.876953353 |
| | 5187 | PER1 | NM_002616 | 202861_at | −0.35662350 | 0.877249218 |
| | 2098 | ESD | AF112219 | 215096_s_at | −0.33165654 | 0.877568889 |
| | 81887 | LAS1L | Contig40237_RC | 208117_s_at | 0.355525467 | 0.878185905 |
| | 1811 | SLC26A3 | NM_000111 | 206143_at | −0.32496995 | 0.878523665 |
| | 54535 | CCHCR1 | NM_019052 | 42361_g_at | 0.303212335 | 0.879290516 |
| | 55526 | DHTKD1 | Contig173 | 209916_at | 0.302461461 | 0.880741229 |
| | 57161 | PELI2 | NM_021255 | 219132_at | −0.34000435 | 0.881182055 |
| | 2353 | FOS | NM_005252 | 209189_at | −0.34853137 | 0.881316836 |
| | 51279 | C1RL | NM_016546 | 218983_at | −0.34801489 | 0.882609 |
| | 60436 | TGIF2 | AF055012 | 218724_s_at | 0.347072353 | 0.883569866 |
| | 3028 | HSD17B10 | NM_004493 | 202282_at | 0.341783943 | 0.88402224 |
| | 26519 | TIMM10 | NM_012456 | 218408_at | 0.342150925 | 0.884715217 |
| | 25960 | GPR124 | AB040964 | 221814_at | −0.33867805 | 0.88492336 |
| | 10252 | SPRY1 | AF041037 | 212558_at | −0.34627190 | 0.885767923 |
| | 6199 | RPS6KB2 | NM_003952 | 203777_s_at | 0.316080366 | 0.885921604 |
| | 9824 | ARHGAP11A | NM_014783 | 204492_at | 0.271468635 | 0.886970555 |
| | 55630 | SLC39A4 | NM_017767 | 219215_s_at | 0.353664658 | 0.887047277 |
| | 7049 | TGFBR3 | NM_003243 | 204731_at | −0.32807103 | 0.887698816 |
| | 8607 | RUVBL1 | NM_003707 | 201614_s_at | 0.268410584 | 0.888152059 |
| | 2581 | GALC | NM_000153 | 204417_at | −0.33728855 | 0.888213228 |
| | 862 | RUNX1T1 | NM_004349 | 205528_s_at | −0.35143858 | 0.88846914 |
| | 8458 | TTF2 | NM_003594 | 204407_at | 0.333371618 | 0.88848286 |
| | 9775 | EIF4A3 | NM_014740 | 201303_at | 0.334470277 | 0.891654944 |
| | 3181 | HNRPA2B1 | NM_002137 | 205292_s_at | 0.334227798 | 0.892344287 |
| | 26039 | SS18L1 | AB014593 | 213140_s_at | 0.31535083 | 0.892395413 |
| | 10580 | SORBS1 | NM_015385 | 218087_s_at | −0.33607143 | 0.892619568 |
| | 7056 | THBD | NM_000361 | 203888_at | −0.30846240 | 0.894985585 |
| | 8322 | FZD4 | NM_012193 | 218665_at | −0.35048586 | 0.895167871 |
| | 1003 | CDH5 | NM_001795 | 204677_at | −0.32733789 | 0.895661116 |
| | 2152 | F3 | NM_001993 | 204363_at | −0.33176999 | 0.895910725 |
| | 55068 | NA | NM_017993 | 219501_at | −0.29959642 | 0.897626597 |
| | 64785 | GINS3 | AL137379 | 218719_s_at | 0.345282183 | 0.898041826 |
| | 79042 | TSEN34 | Contig3597_RC | 218132_s_at | 0.316134089 | 0.898125459 |
| | 8805 | TRIM24 | NM_015905 | 204391_x_at | 0.320229877 | 0.899125295 |
| | 1478 | CSTF2 | NM_001325 | 204459_at | 0.319509099 | 0.900149824 |
| | 1746 | DLX2 | NM_004405 | 207147_at | −0.32079479 | 0.902276681 |
| | 57125 | PLXDC1 | NM_020405 | 219700_at | −0.27855897 | 0.902333798 |
| | 22998 | NA | AB029025 | 212328_at | −0.31356352 | 0.903307846 |
| | 79915 | C17orf41 | Contig36210_RC | 220223_at | 0.298348091 | 0.904268882 |
| | 7026 | NR2F2 | M64497 | 215073_s_at | −0.31788442 | 0.905831798 |
| | 7474 | WNT5A | Contig40434_RC | 213425_at | −0.31039903 | 0.906409867 |
| | 55857 | C20orf19 | NM_018474 | 219961_s_at | −0.33045535 | 0.90691686 |
| | 114625 | ERMAP | NM_018538 | 219905_at | −0.29372548 | 0.907329798 |
| | 8857 | FCGBP | NM_003890 | 203240_at | −0.31144091 | 0.908506651 |
| | 26872 | STEAP1 | NM_012449 | 205542_at | −0.30415820 | 0.909645834 |
| | 7226 | TRPM2 | NM_003307 | 205708_s_at | 0.290916974 | 0.911329018 |
| | 29844 | TFPT | NM_013342 | 218996_at | 0.271529206 | 0.913433463 |
| | 4719 | NDUFS1 | NM_005006 | 203039_s_at | 0.303109253 | 0.915015151 |
| | 4013 | LOH11CR2A | NM_014622 | 210102_at | −0.30279595 | 0.915117797 |
| | 3396 | ICT1 | NM_001545 | 204868_at | 0.292070088 | 0.91536279 |
| | 397 | ARHGDIB | NM_001175 | 201288_at | −0.28431343 | 0.916109977 |
| | 10436 | EMG1 | U72514 | 209233_at | 0.29513303 | 0.91771301 |
| | 51582 | AZIN1 | NM_015878 | 201772_at | 0.28911943 | 0.917927776 |
| | 10598 | AHSA1 | NM_012111 | 201491_at | 0.290857764 | 0.9179611 |
| | 333 | APLP1 | NM_005166 | 209462_at | 0.265203127 | 0.919016116 |
| | 51142 | CHCHD2 | NM_016139 | 217720_at | 0.294292226 | 0.919415001 |
| | 27123 | DKK2 | NM_014421 | 219908_at | −0.28658318 | 0.919956834 |
| | 55020 | NA | NM_017931 | 218272_at | −0.28480702 | 0.922283445 |
| | 23460 | ABCA6 | Contig35210_RC | 217504_at | −0.27426772 | 0.922481847 |
| | 64321 | SOX17 | Contig37354_RC | 219993_at | −0.27801934 | 0.925123949 |
| | 7098 | TLR3 | NM_003265 | 206271_at | −0.27152130 | 0.925325276 |
| | 6338 | SCNN1B | NM_000336 | 205464_at | 0.28820584 | 0.925826366 |
| | 3692 | ITGB4BP | NM_002212 | 210213_s_at | 0.263212244 | 0.926734961 |
| | 10253 | SPRY2 | NM_005842 | 204011_at | −0.28525645 | 0.926765742 |
| | 2669 | GEM | NM_005261 | 204472_at | −0.28050966 | 0.926916522 |
| | 79679 | VTCN1 | Contig52970_RC | 219768_at | −0.26124143 | 0.927139343 |
| | 79618 | HMBOX1 | Contig1982_RC | 219269_at | −0.27039086 | 0.92843197 |
| | 8772 | FADD | NM_003824 | 202535_at | 0.27301337 | 0.93042485 |
| | 9986 | RCE1 | NM_005133 | 205333_s_at | 0.25749527 | 0.930511454 |
| | 58500 | ZNF250 | X16282 | 213858_at | 0.249529287 | 0.93097776 |
| | 11081 | KERA | NM_007035 | 220504_at | −0.32349270 | 0.932434909 |
| | 7064 | THOP1 | NM_003249 | 203235_at | 0.21439195 | 0.932738348 |
| | 55799 | CACNA2D3 | NM_018398 | 219714_s_at | −0.26160430 | 0.932985294 |
| | 49855 | ZNF291 | AL137612 | 209741_x_at | −0.25994490 | 0.933064583 |
| | 54606 | DDX56 | NM_019082 | 217754_at | 0.202591131 | 0.934651171 |
| | 7164 | TPD52L1 | NM_003287 | 203786_s_at | 0.260470913 | 0.934685044 |
| | 80775 | TMEM177 | Contig49309_RC | 218897_at | 0.265363587 | 0.934961966 |
| | 667 | DST | NM_001723 | 204455_at | −0.24839799 | 0.935375903 |
| | 2781 | GNAZ | NM_002073 | 204993_at | 0.258872319 | 0.936532833 |
| | 23464 | GCAT | NM_014291 | 205164_at | 0.251880375 | 0.936847336 |
| | 79763 | ISOC2 | Contig2889_RC | 218893_at | 0.256164207 | 0.936952189 |
| | 4649 | MYO9A | NM_006901 | 219027_s_at | −0.25417332 | 0.93701735 |
| | 53820 | DSCR6 | NM_018962 | 207267_s_at | 0.229254645 | 0.93734872 |
| | 3638 | INSIG1 | NM_005542 | 201625_s_at | 0.284659697 | 0.938726931 |
| | 11171 | STRAP | NM_007178 | 200870_at | 0.252556209 | 0.940118601 |
| | 10992 | SF3B2 | NM_006842 | 200619_at | 0.254492749 | 0.940473638 |
| | 6832 | SUPV3L1 | NM_003171 | 212894_at | 0.253167283 | 0.940890077 |
| | 55922 | NKRF | NM_017544 | 205004_at | 0.237927975 | 0.9421922 |
| | 10557 | RPP38 | NM_006414 | 205562_at | 0.267313355 | 0.943143623 |
| | 3216 | HOXB6 | NM_018952 | 205366_s_at | −0.24536489 | 0.944854741 |
| | 54785 | C17orf59 | NM_017622 | 219417_s_at | −0.23521088 | 0.945554277 |
| | 1933 | EEF1B2 | X60656 | 200705_s_at | −0.23781987 | 0.945587039 |
| | 8161 | COIL | NM_004645 | 203653_s_at | 0.232189669 | 0.945723554 |
| | 594 | BCKDHB | NM_000056 | 213321_at | −0.25979226 | 0.9475144 |
| | 6286 | S100P | NM_005980 | 204351_at | 0.232257446 | 0.948099124 |
| | 3954 | LETM1 | NM_012318 | 218939_at | 0.233460226 | 0.948276398 |
| | 51087 | YBX2 | NM_015982 | 219704_at | 0.196514735 | 0.948900789 |
| | 10953 | TOMM34 | NM_006809 | 201870_at | 0.204607911 | 0.949034891 |
| PLAU | 5328 | PLAU | NM_002658 | 211668_s_at | 1 | 0 |
| | 649 | BMP1 | NM_001199 | 207595_s_at | 0.686303345 | 0.534305465 |
| | 4323 | MMP14 | NM_004995 | 202827_s_at | 0.666244138 | 0.559607929 |
| | 7070 | THY1 | NM_006288 | 208850_s_at | 0.613593172 | 0.627698291 |
| | 1290 | COL5A2 | NM_000393 | 221730_at | 0.570972856 | 0.62999627 |
| | 8038 | ADAM12 | NM_003474 | 202952_s_at | 0.546163691 | 0.662574251 |
| | 23452 | ANGPTL2 | AF007150 | 219514_at | 0.574017552 | 0.66386681 |
| | 4237 | MFAP2 | NM_017459 | 203417_at | 0.573117712 | 0.674166716 |
| | 871 | SERPINH1 | NM_004353 | 207714_s_at | 0.551607834 | 0.675286499 |
| | 1291 | COL6A1 | X15880 | 212091_s_at | 0.553673759 | 0.701177797 |
| | 3671 | ISLR | NM_005545 | 207191_s_at | 0.513171443 | 0.726476697 |
| | 9260 | PDLIM7 | NM_005451 | 214121_x_at | 0.529257266 | 0.735614613 |
| | 55742 | PARVA | NM_018222 | 217890_s_at | 0.483569524 | 0.736339664 |
| | 25903 | OLFML2B | AL050137 | 213125_at | 0.516201362 | 0.740220151 |
| | 6876 | TAGLN | NM_003186 | 205547_s_at | 0.500057895 | 0.748828695 |
| | 5476 | CTSA | NM_000308 | 200661_at | 0.476318761 | 0.763036848 |
| | 5159 | PDGFRB | NM_002609 | 202273_at | 0.475040267 | 0.769821276 |
| | 54587 | MXRA8 | AL050202 | 213422_s_at | 0.437778456 | 0.784354172 |
| | 9180 | OSMR | NM_003999 | 205729_at | 0.433306368 | 0.79490084 |
| | 1281 | COL3A1 | NM_000090 | 201852_x_at | 0.449280663 | 0.806105195 |
| | 26585 | GREM1 | NM_013372 | 218468_s_at | 0.431076597 | 0.806133268 |
| | 2191 | FAP | NM_004460 | 209955_s_at | 0.449475987 | 0.808337233 |
| | 1627 | DBN1 | NM_004395 | 217025_s_at | 0.429269432 | 0.809226482 |
| | 23299 | BICD2 | AB014599 | 209203_s_at | 0.430848727 | 0.813994971 |
| | 51330 | TNFRSF12A | NM_016639 | 218368_s_at | 0.436061674 | 0.821259664 |
| | 7421 | VDR | NM_000376 | 204253_s_at | 0.423203335 | 0.823722546 |
| | 6591 | SNAI2 | Contig1585_RC | 213139_at | 0.409857641 | 0.824381249 |
| | 2037 | EPB41L2 | NM_001431 | 201718_s_at | 0.421951551 | 0.825246889 |
| | 55033 | FKBP14 | NM_017946 | 219390_at | 0.425656347 | 0.827817825 |
| | 4681 | NBL1 | NM_005380 | 201621_at | 0.410725353 | 0.836503012 |
| | 10487 | CAP1 | NM_006367 | 213798_s_at | 0.414551349 | 0.843899961 |
| | 526 | ATP6V1B2 | NM_001693 | 201089_at | 0.385305229 | 0.845387478 |
| | 2050 | EPHB4 | NM_004444 | 216680_s_at | 0.33501482 | 0.850336946 |
| | 9697 | TRAM2 | NM_012288 | 202369_s_at | 0.37440913 | 0.851530018 |
| | 4921 | DDR2 | NM_006182 | 205168_at | 0.37934529 | 0.852102907 |
| | 9945 | GFPT2 | NM_005110 | 205100_at | 0.420846996 | 0.852411188 |
| | 4811 | NID1 | NM_002508 | 202007_at | 0.426030363 | 0.85968909 |
| | 8481 | OFD1 | NM_003611 | 203569_s_at | −0.33640817 | 0.875372065 |
| | 23705 | IGSF4 | NM_014333 | 209030_s_at | 0.326615812 | 0.877277896 |
| | 23166 | STAB1 | AJ275213 | 204150_at | 0.345752035 | 0.879137539 |
| | 8459 | TPST2 | NM_003595 | 204079_at | 0.292694524 | 0.879236195 |
| | 23645 | PPP1R15A | NM_014330 | 202014_at | 0.334435453 | 0.88314905 |
| | 27295 | PDLIM3 | NM_014476 | 209621_s_at | 0.344670867 | 0.885652512 |
| | 93974 | ATPIF1 | NM_016311 | 218671_s_at | −0.32802985 | 0.886105389 |
| | 51592 | TRIM33 | NM_015906 | 212435_at | −0.33038360 | 0.895125804 |
| | 4314 | MMP3 | NM_002422 | 205828_at | 0.304242677 | 0.895658603 |
| | 1833 | EPYC | NM_004950 | 206439_at | 0.337308341 | 0.895915378 |
| | 157567 | ANKRD46 | U79297 | 212731_at | −0.32344971 | 0.898025232 |
| | 8904 | CPNE1 | NM_003915 | 206918_s_at | 0.318038406 | 0.900793856 |
| | 602 | BCL3 | NM_005178 | 204907_s_at | 0.304998235 | 0.904399401 |
| | 2720 | GLB1 | NM_000404 | 201576_s_at | 0.322062138 | 0.906764094 |
| | 59286 | UBL5 | Contig65670_RC | 218011_at | −0.27021325 | 0.914865462 |
| | 8408 | ULK1 | NM_003565 | 209333_at | 0.27421269 | 0.918353875 |
| | 55035 | NOL8 | NM_017948 | 218244_at | −0.27456644 | 0.922310693 |
| | 7042 | TGFB2 | NM_003238 | 220407_s_at | 0.286360255 | 0.923466436 |
| | 5155 | PDGFB | NM_002608 | 204200_s_at | 0.269055708 | 0.931600028 |
| | 10409 | BASP1 | NM_006317 | 202391_at | 0.244062133 | 0.932183339 |
| | 10993 | SDS | NM_006843 | 205695_at | 0.245388394 | 0.933091037 |
| | 6233 | RPS27A | NM_002954 | 200017_at | −0.26468902 | 0.933902258 |
| | 8507 | ENC1 | NM_003633 | 201340_s_at | 0.230967436 | 0.934843627 |
| | 176 | AGC1 | NM_013227 | 217161_x_at | 0.214527206 | 0.938418486 |
| | 9849 | ZNF518 | NM_014803 | 204291_at | −0.27940542 | 0.941723169 |
| | 51463 | GPR89A | NM_016334 | 222140_s_at | −0.24633996 | 0.942684028 |
| | 6141 | RPL18 | NM_000979 | 222297_x_at | −0.24477092 | 0.944074771 |
| | 4205 | MEF2A | NM_005587 | 208328_s_at | 0.206794876 | 0.9444056 |
| | 1774 | DNASE1L1 | NM_006730 | 203912_s_at | 0.232623402 | 0.946207309 |
| | 4430 | MYO1B | AK000160 | 212364_at | 0.228075133 | 0.947362794 |
| | 57158 | JPH2 | NM_020433 | 220385_at | 0.163350482 | 0.949439143 |
| VEGF | 7422 | VEGFA | NM_003376 | 211527_x_at | 1 | 0 |
| | 911 | CD1C | NM_001765 | 205987_at | −0.30279189 | 0.875335287 |
| | 4005 | LMO2 | NM_005574 | 204249_s_at | −0.35419700 | 0.876731359 |
| | 4222 | MEOX1 | NM_013999 | 205619_s_at | −0.35048957 | 0.882751646 |
| | 29927 | SEC61A1 | NM_013336 | 217716_s_at | 0.348075751 | 0.885518246 |
| | 6166 | RPL36AL | NM_001001 | 207585_s_at | −0.33751206 | 0.887065036 |
| | 9450 | LY86 | NM_004271 | 205859_at | −0.29401754 | 0.907178982 |
| | 22900 | CARD8 | NM_014959 | 204950_at | −0.29984162 | 0.912490569 |
| | 1776 | DNASE1L3 | NM_004944 | 205554_s_at | −0.29876991 | 0.915582301 |
| | 1119 | CHKA | NM_001277 | 204233_s_at | 0.293232546 | 0.918063311 |
| | 22809 | ATF5 | NM_012068 | 204999_s_at | 0.217042464 | 0.937083889 |
| | 23417 | MLYCD | NM_012213 | 218869_at | −0.23534131 | 0.939494944 |
| | 23592 | LEMD3 | NM_014319 | 218604_at | −0.26982318 | 0.947647276 |
| | 51621 | KLF13 | NM_015995 | 219878_s_at | 0.242003861 | 0.947879938 |
| STAT1 | 6772 | STAT1 | NM_007315 | 209969_s_at | 1 | 0 |
| | 3627 | CXCL10 | NM_001565 | 204533_at | 0.791673192 | 0.373734657 |
| | 6890 | TAP1 | NM_000593 | 202307_s_at | 0.773730642 | 0.38014378 |
| | 6373 | CXCL11 | NM_005409 | 210163_at | 0.729976561 | 0.469038038 |
| | 3620 | INDO | NM_002164 | 210029_at | 0.693332241 | 0.480540278 |
| | 4283 | CXCL9 | NM_002416 | 203915_at | 0.705931141 | 0.506582671 |
| | 4599 | MX1 | NM_002462 | 202086_at | 0.700341707 | 0.512026803 |
| | 27074 | LAMP3 | NM_014398 | 205569_at | 0.691286706 | 0.51665141 |
| | 9636 | ISG15 | NM_005101 | 205483_s_at | 0.692921839 | 0.521514816 |
| | 64108 | RTP4 | Contig51660_RC | 219684_at | 0.66510774 | 0.521724062 |
| | 55008 | HERC6 | NM_017912 | 219352_at | 0.680045765 | 0.534540502 |
| | 10964 | IFI44L | NM_006820 | 204439_at | 0.68441612 | 0.53484654 |
| | 4600 | MX2 | M30818 | 204994_at | 0.676333667 | 0.545187222 |
| | 3437 | IFIT3 | NM_001549 | 204747_at | 0.676843523 | 0.547342002 |
| | 51191 | HERC5 | NM_016323 | 219863_at | 0.654162297 | 0.55158659 |
| | 91543 | RSAD2 | AF026941 | 213797_at | 0.654314865 | 0.566762715 |
| | 23586 | DDX58 | NM_014314 | 218943_s_at | 0.640872007 | 0.568844077 |
| | 6352 | CCL5 | NM_002985 | 1405_i_at | 0.660200416 | 0.568867672 |
| | 27299 | ADAMDEC1 | NM_014479 | 206134_at | 0.642299127 | 0.589527746 |
| | 914 | CD2 | NM_001767 | 205831_at | 0.644301271 | 0.616877785 |
| | 55601 | NA | NM_017631 | 218986_s_at | 0.613852226 | 0.621928407 |
| | 10866 | HCP5 | NM_006674 | 206082_at | 0.610103583 | 0.629169819 |
| | 9111 | NMI | NM_004688 | 203964_at | 0.603257958 | 0.639437655 |
| | 9806 | SPOCK2 | NM_014767 | 202524_s_at | 0.584098575 | 0.641216629 |
| | 6355 | CCL8 | NM_005623 | 214038_at | 0.570756407 | 0.651950505 |
| | 10346 | TRIM22 | NM_006074 | 213293_s_at | 0.590810894 | 0.652849087 |
| | 4069 | LYZ | NM_000239 | 213975_s_at | 0.544927822 | 0.662182124 |
| | 3659 | IRF1 | NM_002198 | 202531_at | 0.589919529 | 0.66222688 |
| | 3902 | LAG3 | NM_002286 | 206486_at | 0.541977347 | 0.668358145 |
| | 9595 | PSCDBP | NM_004288 | 209606_at | 0.567980838 | 0.668469879 |
| | 22797 | TFEC | NM_012252 | 206715_at | 0.599293976 | 0.668483201 |
| | 10537 | UBD | NM_006398 | 205890_s_at | 0.578544702 | 0.670772877 |
| | 11262 | SP140 | NM_007237 | 207777_s_at | 0.577805009 | 0.679232612 |
| | 1075 | CTSC | NM_001814 | 201487_at | 0.562320779 | 0.681366545 |
| | 2537 | IFI6 | NM_002038 | 204415_at | 0.563222465 | 0.683899859 |
| | 7941 | PLA2G7 | NM_005084 | 206214_at | 0.557200093 | 0.695642543 |
| | 917 | CD3G | NM_000073 | 206804_at | 0.55769671 | 0.698961356 |
| | 1890 | ECGF1 | NM_001953 | 204858_s_at | 0.546473637 | 0.700870238 |
| | 51316 | PLAC8 | NM_016619 | 219014_at | 0.538438452 | 0.703113148 |
| | 10875 | FGL2 | NM_006682 | 204834_at | 0.524540085 | 0.705303623 |
| | 3003 | GZMK | NM_002104 | 206666_at | 0.530074132 | 0.717735405 |
| | 962 | CD48 | NM_001778 | 204118_at | 0.533233612 | 0.719024509 |
| | 6775 | STAT4 | NM_003151 | 206118_at | 0.550392357 | 0.72324098 |
| | 2841 | GPR18 | Contig35647_RC | 210279_at | 0.521231488 | 0.726949329 |
| | 5026 | P2RX5 | NM_002561 | 210448_s_at | 0.504830283 | 0.729589032 |
| | 10437 | IFI30 | NM_006332 | 201422_at | 0.511822231 | 0.735812254 |
| | 4068 | SH2D1A | NM_002351 | 210116_at | 0.471245594 | 0.7433416 |
| | 7805 | LAPTM5 | NM_006762 | 201720_s_at | 0.498421145 | 0.746819193 |
| | 969 | CD69 | NM_001781 | 209795_at | 0.471158768 | 0.753189587 |
| | 5778 | PTPN7 | NM_002832 | 204852_s_at | 0.499057802 | 0.75677133 |
| | 3394 | IRF8 | NM_002163 | 204057_at | 0.489162341 | 0.768389511 |
| | 11040 | PIM2 | NM_006875 | 204269_at | 0.47698737 | 0.770321793 |
| | 51513 | ETV7 | NM_016135 | 221680_s_at | 0.532716749 | 0.771749503 |
| | 29909 | GPR171 | NM_013308 | 207651_at | 0.467045116 | 0.776788947 |
| | 5720 | PSME1 | NM_006263 | 200814_at | 0.463856614 | 0.778162143 |
| | 330 | BIRC3 | NM_001165 | 210538_s_at | 0.47318545 | 0.778456521 |
| | 356 | FASLG | NM_000639 | 210865_at | 0.521488064 | 0.782352474 |
| | 8519 | IFITM1 | NM_003641 | 201601_x_at | 0.469088027 | 0.78238098 |
| | 24138 | IFIT5 | NM_012420 | 203596_s_at | 0.466667589 | 0.783188342 |
| | 3689 | ITGB2 | NM_000211 | 202803_s_at | 0.461692343 | 0.784532984 |
| | 11118 | BTN3A2 | NM_007047 | 212613_at | 0.461680236 | 0.788500748 |
| | 3059 | HCLS1 | NM_005335 | 202957_at | 0.450361209 | 0.795023723 |
| | 6398 | SECTM1 | NM_003004 | 213716_s_at | 0.425961617 | 0.799831467 |
| | 55843 | ARHGAP15 | NM_018460 | 218870_at | 0.417535994 | 0.801382989 |
| | 22914 | KLRK1 | NM_007360 | 205821_at | 0.437660493 | 0.809727352 |
| | 10261 | IGSF6 | NM_005849 | 206420_at | 0.436549677 | 0.81219172 |
| | 1880 | EBI2 | NM_004951 | 205419_at | 0.399159019 | 0.815726925 |
| | 26034 | NA | AB007863 | 214735_at | 0.40937931 | 0.829560298 |
| | 29887 | SNX10 | NM_013322 | 218404_at | 0.400589724 | 0.835603896 |
| | 79132 | NA | Contig63102_RC | 219364_at | 0.391375097 | 0.849609415 |
| | 684 | BST2 | NM_004335 | 201641_at | 0.384303271 | 0.854129545 |
| | 55337 | NA | NM_018381 | 218429_s_at | 0.386327296 | 0.857355054 |
| | 341 | APOC1 | NM_001645 | 204416_x_at | 0.36462583 | 0.861296021 |
| | 51237 | NA | NM_016459 | 221286_s_at | 0.370554593 | 0.874957917 |
| | 445347 | NA | M17323 | 209813_x_at | 0.305107684 | 0.886124869 |
| | 56829 | ZC3HAV1 | NM_020119 | 220104_at | 0.342023355 | 0.888935417 |
| | 23564 | DDAH2 | NM_013974 | 214909_s_at | −0.33358568 | 0.889200466 |
| | 23547 | LILRA4 | AF041261 | 210313_at | 0.341444621 | 0.894341374 |
| | 10148 | EBI3 | NM_005755 | 219424_at | 0.284618325 | 0.894479773 |
| | 3823 | KLRC3 | NM_007333 | 207723_s_at | 0.269791167 | 0.896638494 |
| | 50856 | CLEC4A | NM_016184 | 221724_s_at | 0.348085505 | 0.90159803 |
| | 959 | CD40LG | NM_000074 | 207892_at | 0.330319064 | 0.90731366 |
| | 7409 | VAV1 | NM_005428 | 206219_s_at | 0.346468277 | 0.907387687 |
| | 2745 | GLRX | NM_002064 | 206662_at | 0.30616967 | 0.910310197 |
| | 54 | ACP5 | NM_001611 | 204638_at | 0.276526368 | 0.911099185 |
| | 5993 | RFX5 | NM_000449 | 202964_s_at | 0.292677164 | 0.911410075 |
| | 51816 | CECR1 | NM_017424 | 219505_at | 0.305675892 | 0.913657631 |
| | 7187 | TRAF3 | NM_003300 | 208315_x_at | 0.246604319 | 0.921975101 |
| | 4218 | RAB8A | NM_005370 | 208819_at | 0.272692263 | 0.923395016 |
| | 3606 | IL18 | NM_001562 | 206295_at | 0.265963985 | 0.927706943 |
| | 1942 | EFNA1 | NM_004428 | 202023_at | −0.25887098 | 0.934754499 |
| | 10125 | RASGRP1 | NM_005739 | 205590_at | 0.256021016 | 0.936422237 |
| | 9985 | REC8L1 | NM_005132 | 218599_at | 0.258614123 | 0.936428333 |
| | 9034 | CCRL2 | NM_003965 | 211434_s_at | 0.318651272 | 0.940353226 |
| | 10126 | DNAL4 | NM_005740 | 204008_at | −0.21990042 | 0.943877702 |
| CASP3 | 836 | CASP3 | NM_004346 | 202763_at | 1 | 0 |
| | 10393 | ANAPC10 | NM_014885 | 207845_s_at | 0.356889908 | 0.902909966 |
| | 7738 | ZNF184 | U66561 | 213452_at | 0.2920488 | 0.913630754 |
| | 3728 | JUP | NM_002230 | 201015_s_at | −0.27257126 | 0.924223529 |
| | 8237 | USP11 | NM_004651 | 208723_at | −0.29065181 | 0.925692835 |
| | 402 | ARL2 | NM_001667 | 202564_x_at | −0.25533419 | 0.935253954 |
| | 25978 | CHMP2B | NM_014043 | 202536_at | 0.265905131 | 0.937256343 |
| | 6301 | SARS | NM_006513 | 200802_at | −0.25179738 | 0.937862493 |
| | 55361 | NA | AL353952 | 209346_s_at | −0.24294692 | 0.943220971 |
| | 5977 | DPF2 | NM_006268 | 202116_at | −0.21593926 | 0.947438324 |

TABLE 10

| gene.symbol | EntrezGene.ID |
|---|---|
| ALPI | 248 |
| ANPEP | 290 |
| ARHGDIB | 397 |
| BAG4 | 9530 |
| BAX | 581 |
| BBS9 | 27241 |
| BID | 637 |
| BIRC3 | 330 |
| BLVRA | 644 |
| C17orf46 | 124783 |
| CASP10 | 843 |
| CASP6 | 839 |
| CASP8 | 841 |
| CASP9 | 842 |
| CD28 | 940 |
| CD33 | 945 |
| CD4 | 920 |
| CD40 | 958 |
| CD44 | 960 |
| CD5 | 921 |
| CD7 | 924 |
| CD80 | 941 |
| CD86 | 942 |
| CFLAR | 8837 |
| CR2 | 1380 |
| CRADD | 8738 |
| CSNK1D | 1453 |
| CUTL1 | 1523 |
| CYCS | 54205 |
| DAXX | 1616 |
| EIF4A1 | 1973 |
| EIF4E | 1977 |
| ELK1 | 2002 |
| FAF1 | 11124 |
| FAS | 355 |
| FKBP1A | 2280 |
| GRB2 | 2885 |
| HLA-A | 3105 |
| HLA-DRB1 | 3123 |
| HLA-DRB5 | 3127 |
| ICAM1 | 3383 |
| ICOSLG | 23308 |
| IKBKB | 3551 |
| IL10RA | 3587 |
| IL12B | 3593 |
| IL12RB2 | 3595 |
| IL13 | 3596 |
| IL15 | 3600 |
| IL1A | 3552 |
| IL2RA | 3559 |
| IL3 | 3562 |
| IL4R | 3566 |
| IRAK2 | 3656 |
| ITGA4 | 3676 |
| ITGAM | 3684 |
| ITGAX | 3687 |
| ITK | 3702 |
| JAK1 | 3716 |
| JAK3 | 3718 |
| JUNB | 3726 |
| LMNA | 4000 |
| LMNB1 | 4001 |
| LTA | 4049 |
| MADD | 8567 |
| MAF | 4094 |
| MAP2K3 | 5606 |
| MAP3K14 | 9020 |
| MAP3K7IP1 | 10454 |
| MAP4K2 | 5871 |
| MAPK1 | 5594 |
| MAPK8 | 5599 |
| MYD88 | 4615 |
| NCF2 | 4688 |
| NFKB1 | 4790 |
| NR3C1 | 2908 |
| NSMAF | 8439 |
| PAK2 | 5062 |
| PDK2 | 5164 |
| PIK3C2G | 5288 |
| PLCB1 | 23236 |
| PPP1R13B | 23368 |
| PPP3CA | 5530 |
| PRF1 | 5551 |
| PRKAR1B | 5575 |
| PRKDC | 5591 |
| PTEN | 5728 |
| PTENP1 | 11191 |
| PTPRC | 5788 |
| PVRL1 | 5818 |
| RAF1 | 5894 |
| RELA | 5970 |
| RHEB | 6009 |
| RPS6KB1 | 6198 |
| SPTAN1 | 6709 |
| STAT3 | 6774 |
| STAT5A | 6776 |
| TANK | 10010 |
| TAP1 | 6890 |
| TAP2 | 6891 |
| TGFB1 | 7040 |
| TNF | 7124 |
| TNFRSF10A | 8797 |
| TNFRSF13B | 23495 |
| TNFRSF1B | 7133 |
| TNFRSF25 | 8718 |
| TNFSF13B | 10673 |
| TOLLIP | 54472 |
| TRA@ | 6955 |
| TRAF1 | 7185 |
| TRAF3 | 7187 |

TABLE 11

| gene.symbol | EntrezGene.ID |
|---|---|
| ACP5 | 54 |
| ADAMDEC1 | 27299 |
| APOC1 | 341 |
| ARHGAP15 | 55843 |
| BIRC3 | 330 |
| BST2 | 684 |
| BTN3A2 | 11118 |
| CCL5 | 6352 |
| CCL8 | 6355 |
| CCRL2 | 9034 |
| CD2 | 914 |
| CD3G | 917 |
| CD40LG | 959 |
| CD48 | 962 |
| CD69 | 969 |
| CECR1 | 51816 |
| CLEC4A | 50856 |
| CTSC | 1075 |
| CXCL10 | 3627 |
| CXCL11 | 6373 |
| CXCL9 | 4283 |
| DDAH2 | 23564 |
| DDX58 | 23586 |
| DNAL4 | 10126 |
| EBI2 | 1880 |
| EBI3 | 10148 |
| ECGF1 | 1890 |
| EFNA1 | 1942 |
| ETV7 | 51513 |
| FASLG | 356 |
| FGL2 | 10875 |
| FLJ11286 | 55337 |
| FLJ20035 | 55601 |
| GLRX | 2745 |
| GPR171 | 29909 |
| GPR18 | 2841 |
| GZMK | 3003 |
| HCLS1 | 3059 |
| HCP5 | 10866 |
| HERC5 | 51191 |
| HERC6 | 55008 |
| IFI30 | 10437 |
| IFI44L | 10964 |
| IFI6 | 2537 |
| IFIT3 | 3437 |
| IFIT5 | 24138 |
| IFITM1 | 8519 |
| IGSF6 | 10261 |
| IL18 | 3606 |
| INDO | 3620 |
| IRF1 | 3659 |
| IRF8 | 3394 |
| ISG15 | 9636 |
| ITGB2 | 3689 |
| KLRC3 | 3823 |
| KLRK1 | 22914 |
| LAG3 | 3902 |
| LAMP3 | 27074 |
| LAPTM5 | 7805 |
| LGP2 | 79132 |
| LILRA4 | 23547 |
| LILRB1 | 10859 |
| MGC29506 | 51237 |
| MX1 | 4599 |
| MX2 | 4600 |
| NMI | 9111 |
| P2RX5 | 5026 |
| PIM2 | 11040 |
| PIP3-E | 26034 |
| PLA2G7 | 7941 |
| PLAC8 | 51316 |
| PSCDBP | 9595 |
| PSME1 | 5720 |
| PTPN7 | 5778 |
| RAB8A | 4218 |
| RASGRP1 | 10125 |
| REC8L1 | 9985 |
| RFX5 | 5993 |
| RSAD2 | 91543 |
| RTP4 | 64108 |
| SECTM1 | 6398 |
| SH2D1A | 4068 |
| SNX10 | 29887 |
| SP140 | 11262 |
| SPOCK2 | 9806 |
| STAT1 | 6772 |
| STAT4 | 6775 |
| TAP1 | 6890 |
| TFEC | 22797 |
| TRAF3 | 7187 |
| TRGV9 | 6983 |
| TRIM22 | 10346 |
| UBD | 10537 |
| VAV1 | 7409 |
| ZC3HAV1 | 56829 |

TABLE 12

| gene.symbol | EntrezGene.ID |
|---|---|
| FGD6 | 55785 |
| PLAC9 | 219348 |
| CAB39L | 81617 |
| FGD6 | 55785 |
| LONRF3 | 79836 |
| CGI-38 | 51673 |
| STXBP6 | 29091 |
| FHL1 | 2273 |
| STXBP6 | 29091 |
| LEPR | 3953 |
| CA4 | 762 |
| TNMD | 64102 |
| POSTN | 10631 |
| LOC58489 | 58489 |
| LOC284825 | 284825 |
| LRP1B | 53353 |
| TIMP4 | 7079 |
| STXBP6 | 29091 |
| WNT11 | 7481 |
| PLAC9 | 219348 |
| MICAL2 | 9645 |
| PKD1L2 | 114780 |
| SDC1 | 6382 |
| FHL1 | 2273 |
| FHL1 | 2273 |
| F2RL2 | 2151 |
| AKR1C2 | 1646 |
| LEF1 | 51176 |
| ADAM12 | 8038 |
| ADH1C | 126 |
| VIT | 5212 |
| HOP | 84525 |
| GPX3 | 2878 |
| RRM2 | 6241 |
| GPX3 | 2878 |
| MYOC | 4653 |
| CLEC3B | 7123 |
| GRP | 2922 |
| GJB2 | 2706 |
| AADAC | 13 |
| MATN3 | 4148 |
| PPAPDC1A | 196051 |
| LOC646324 | 646324 |
| COL10A1 | 1300 |
| COL10A1 | 1300 |

TABLE 13

| gene.symbol | EntrezGene.ID |
|---|---|
| PLAU | 5328 |
| BMP1 | 649 |
| MMP14 | 4323 |
| THY1 | 7070 |
| COL5A2 | 1290 |
| ADAM12 | 8038 |
| ANGPTL2 | 23452 |
| MFAP2 | 4237 |
| SERPINH1 | 871 |
| COL6A1 | 1291 |
| ISLR | 3671 |
| PDLIM7 | 9260 |
| PARVA | 55742 |
| OLFML2B | 25903 |
| TAGLN | 6876 |
| CTSA | 5476 |
| PDGFRB | 5159 |
| MXRA8 | 54587 |
| OSMR | 9180 |
| COL3A1 | 1281 |
| GREM1 | 26585 |
| FAP | 2191 |
| DBN1 | 1627 |
| BICD2 | 23299 |
| TNFRSF12A | 51330 |
| VDR | 7421 |
| SNAI2 | 6591 |
| EPB41L2 | 2037 |
| FKBP14 | 55033 |
| NBL1 | 4681 |
| CAP1 | 10487 |
| ATP6V1B2 | 526 |
| EPHB4 | 2050 |
| TRAM2 | 9697 |
| DDR2 | 4921 |
| GFPT2 | 9945 |
| NID1 | 4811 |
| OFD1 | 8481 |
| CADM1 | 23705 |
| STAB1 | 23166 |
| TPST2 | 8459 |
| PPP1R15A | 23645 |
| PDLIM3 | 27295 |
| ATPIF1 | 93974 |
| TRIM33 | 51592 |
| MMP3 | 4314 |
| EPYC | 1833 |
| ANKRD46 | 157567 |
| CPNE1 | 8904 |
| BCL3 | 602 |
| GLB1 | 2720 |
| UBL5 | 59286 |
| ULK1 | 8408 |
| NOL8 | 55035 |
| TGFB2 | 7042 |
| PDGFB | 5155 |
| BASP1 | 10409 |
| SDS | 10993 |
| RPS27A | 6233 |
| ENC1 | 8507 |
| ACAN | 176 |
| ZNF518 | 9849 |
| GPR89A | 51463 |
| RPL18 | 6141 |
| MEF2A | 4205 |
| DNASE1L1 | 1774 |
| MYO1B | 4430 |
| JPH2 | 57158 |

### REFERENCES

1. Desmedt, C. and Sotiriou, C. Cell Cycle, 5: 2198-2202, 2006.
2. Galon, J. et al. Science, 313: 1960-1964, 2006.
3. Bates, G. J. et al. J. Clin. Oncol., 24: 5373-5380, 2006.
4. van de Vijver, M. et al. N. Engl. J. Med., 347: 1999-2009, 2002.
5. Buyse, M. et al. J. Natl. Cancer Inst., 98: 1183-1192, 2006.
6. Loi, S. et al. J. Clin. Oncol., 25: 1239-1246, 2007.
7. Sotiriou, C. et al. Proc. Natl. Acad. Sci. U.S.A, 100: 10393-10398, 2003.
8. Miller, L. D. et al. Proc. Natl. Acad. Sci. U.S.A, 102: 13550-13555, 2005.
9. Sotiriou, C. et al. J. Natl. Cancer Inst., 98: 262-272, 2006.
10. 't Veer, L. J. et al. Nature, 415: 530-536, 2002.
11. Sorlie, T. et al. Proc. Natl. Acad. Sci. U.S.A, 100: 8418-8423, 2003.
12. Chang, H. Y. et al. PLoS. Biol., 2: E7, 2004.
13. Liu, R. et al. N. Engl. J. Med., 356: 217-226, 2007.
14. Paik, S. et al. N. Engl. J. Med., 351: 2817-2826, 2004.
15. 't Veer, L. J. et al. Breast Cancer Res., 5: 57-58, 2003.
16. Wang Y, et al. Lancet 2005, 365, 671-679.
17. Foekens J A, et al. J. Clin Oncol 2006, 24, 1665-1671
18. Chang H Y, et al. Proc Natl Acad Sci USA 2005, 102, 3738-3743.
19. Maglott D, et al. Nucleic acids research 2007 Database issue): D26-31.
20. Shi L, et al. Nat. Biotechnol. 2006, 9, 1151-61.
21. S. Chen and S. A. Billings and W. Luo. Proc Natl Acad Sci USA 1989, 30, 1873-1896.
22. Allen D M. Technometrics 1974, 19, 125-127.
23. McLachlan G and Peel D (2000) Finite Mixture Models, J. Wiley and Sons, 419 p.
24. G. Schwarz. Estimating the dimension of a model, Annals of Statistics 1978, 6, 461-464.
25. W. G. Cochrane Problems arising in the analysis of a series of similar experiments, Journal of the Royal Statistical Society 1937, 4, 102-118.
26. Desmedt C. Clin Cancer Res 2007, 13, 3207-3214
27. Perou C M, et al. Nature 2000, 406, 747-752.
28. Sorlie T, et al. Proc Natl Acad Sci USA 2001, 98, 10869-10874.
29. Sorlie T, et al. Proc Natl Acad Sci USA 2003, 100, 8418-8423.
30. Sotiriou C, et al. Proc Natl Acad Sci USA 2003, 100, 10393-10398.
31. Remvikos Y. Breast Cancer Res Treat 1995, 34, 25-33.
32. Kaptain S. Diagn Mol Pathol 2001, 10, 139-152.
33. Hu J C. Eur J Surg Oncol 2001, 27, 335-337.
34. Ellis M J, et al. J Clin Oncol 2001, 19, 3808-3816.
35. Ellis M J, et al. J Clin Oncol 2006, 24, 3019-3025.
36. Smith I E, et al. J. Clin. Oncol, 23, 5108-5116.
37. Lal P. Am J Clin Pathol 2005, 123, 541-546.
38. Leissner P, et al. BMC Cancer 2006, 31, 6:216.
39. Bolat F, et al. J Exp Clin Cancer Res 2006, 3, 365-372.
40. Widschwendter A, et al. Clin Cancer Res 2002; 8, 3065-3074.
41. Kapp A V, et al. BMC Genomics 2006, 7:231.
42. Urban P, et al. J Clin Oncol 2006, 24, 4245-4253.
43. Rouzier R, et al. Clin Cancer Res 2005, 11, 5678-5685.
44. Carey L A, et al. Clin Cancer Res 2007, 13, 2329-2334.
45. Kennedy R D. J Natl Cancer Inst 2004, 96, 1659-1668.
46. Muhlethaler-Mottet A. Immunity 1998, 8, 157-166.
47. Lynch R A. Cancer Res 2007, 67, 1254-1261.
48. Colozza M, et al. Ann Oncol 2005, 11, 1723-1739.
49. Ma X J, et al. Cancer cell 2004, 6, 607-616
50. Pawitan Y, et al. Breast Cancer Res 2005, 6, R953-964.
51. Oh D S, et al. J Clin Oncol 2006, 24, 1656-1664.

1. A gene or protein set consisting of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, and possibly 40, 45, 50, 55, 60, 65 genes or proteins or the entire set selected from the table 12 and/or the table 13, antibodies or hypervariable portion thereof directed against the proteins encoded by these genes.

2. The gene or protein set according to claim 1, wherein the gene or proteins sequences or the antibodies are bound to a solid support surface, such as an array.

3. Diagnostic kit or device comprising the gene or protein set according to claim 1 and other means for real time PCR analysis or protein analysis.

4. The kit or device according to claim 3, wherein the means for real time PCR are means for qRT-PCR.

5. The kit or device according to claim 3, which further comprises a gene or protein set consisting of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95 possibly 100, 105, 110 genes or proteins or the entire set selected from the table 10 and/or the table 11, antibodies or hypervariable portion thereof directed against the proteins encoded by these genes.

6. The kit or device according to claim 3, which further comprises a gene or protein set consisting of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90 or 95 genes or proteins or the entire set designated as upregulated genes/proteins in grade 3 tumor in ER+ patients in the table 3 of the document WO 2006/119593 antibodies or hypervariable portion thereof directed against the proteins encoded by these genes.

7. The kit or device according to the claim 6, wherein the genes are proliferation relating genes, preferably selected from the group consisting of CCNB1, CCNA2, CDC2, CDC20, MCM2, MYBL2, KPNA2 and STK6.

8. The kit or device according to claim 3, which further comprises one or more reference genes selected from the group consisting of TFRC, GUS, RPLPO and TBP.

9. The kit or device according to claim 1 comprising a computerized system comprising a bio-assay module configured for detecting a gene expression or protein analysis from a tumor sample based upon the gene or protein set according to claim 1 and a processor module configured to calculate expression of the gene or the protein synthesis and to generate a risk assessment for the tumor sample.

10. The kit or device according to the claim 9, wherein the tumor sample is a breast tumor sample.

11. A gene or protein set consisting of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90 or 95 or proteins or the entire set selected from the table 11 and/or the table 13 or antibodies or hypervariable portion thereof directed against the proteins encoded by these genes.

12. A method for a prognosis (prognostic) of cancer in mammal subject, which comprises the step of collecting a tumor sample, preferably a breast tumor sample, from the mammal subject and measuring gene expression in the tumor sample by putting and measuring gene expression or protein synthesis in the tumor sample by putting into contact nucleotide and/or amino acids sequences obtained from this tumor sample with the gene or protein set of claim 1 and generating a risk assessment for the tumor sample as different subtypes within ER− type and within HER2+ and/or ER+ types.

13. A gene or protein set comprising at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, and possibly 40, 45, 50, 55, 60, 65 genes or proteins or the entire set selected from the table 12 and/or the table 13, antibodies or hypervariable portion thereof directed against the proteins encoded by these genes.

14. The kit or device according to claim 3, which further comprises a gene or protein set comprising at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95 possibly 100, 105, 110 genes or proteins or the entire set selected from the table 10 and/or the table 11, antibodies or hypervariable portion thereof directed against the proteins encoded by these genes.

15. The kit or device according to claim 3, which further comprises a gene or protein set comprising at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90 or 95 genes or proteins or the entire set designated as upregulated genes/proteins in grade 3 tumor in ER+ patients in the table 3 of the document WO 2006/119593 antibodies or hypervariable portion thereof directed against the proteins encoded by these genes.

16. The kit or device according to claim 6, wherein the genes are proliferation relating genes, preferably selected from the group consisting of the gene CDC2, CDC20, MYBL2 and KPNA2.

17. A method for a prognosis (prognostic) of cancer in mammal subject according to claim 12 wherein the subject comprises an ER− human patient.
